SEQUENCE LISTING (582 sequences)

SEQ ID NO: 1 - protein, 10 aa, Arabidopsis thaliana
MLCVLQGLRE

SEQ ID NO: 2 - protein, 17 aa, Arabidopsis thaliana
MIIINNDNYLLLFYNNN

SEQ ID NO: 3 - protein, 13 aa, Arabidopsis thaliana
MIIIYYCFIIIIN

SEQ ID NO: 4 - protein, 11 aa, Arabidopsis thaliana
MLMYRMRSGAN

SEQ ID NO: 5 - protein, 18 aa, Arabidopsis thaliana
MGLNEDSVFRSIKPFKSP

SEQ ID NO: 6 - protein, 39 aa, Arabidopsis thaliana
MNVLARAPRLRHQLQNLTQDRRKTQMQMKGQRVRTTSLQ

SEQ ID NO: 7 - protein, 21 aa, Arabidopsis thaliana
MAREEKEQSVYDIYTFASLLL

SEQ ID NO: 8 - protein, 23 aa, Arabidopsis thaliana
MHCLLSLLEMVPSNLLSALLNLN

SEQ ID NO: 9 - protein, 26 aa, Arabidopsis thaliana
MSVEICRRYYLPPPNNCTYTTPQTSI

SEQ ID NO: 10 - protein, 13 aa, Arabidopsis thaliana
MICYKNLARQNIN

SEQ ID NO: 11 - protein, 10 aa, Arabidopsis thaliana
MSSYQSIYEC

SEQ ID NO: 12 - protein, 14 aa, Arabidopsis thaliana
MIHIHINWRLIRIG

SEQ ID NO: 13 - protein, 24 aa, Arabidopsis thaliana
MHIYALANSNQSPINVYMYHSHRP

SEQ ID NO: 14 - protein, 21 aa, Arabidopsis thaliana
MCICIIHIGPRLILTILQYVF

SEQ ID NO: 15 - protein, 75 aa, Arabidopsis thaliana
MSVKKRALESKNNGSPKSSHYNRCLSFSFLESSTGEKKKPSSLNRMDSKILKAEIVKWAKRVAAYARQLSSRKQD

SEQ ID NO: 16 - protein, 13 aa, Arabidopsis thaliana
MKLVQADPFSDCF

SEQ ID NO: 17 - protein, 30 aa, Arabidopsis thaliana
MEAPSRVIIIDACHSRSWSHQRERRRNHHR

SEQ ID NO: 18 - protein, 13 aa, Arabidopsis thaliana
MLVNLARENKIKT

SEQ ID NO: 19 - protein, 25 aa, Arabidopsis thaliana
MKKAYGKIDMHSFYIDLGPHSFNHS

SEQ ID NO: 20 - protein, 33 aa, Arabidopsis thaliana
MLVILVLGVINGREEETIIVEPYGFQNTQGRDR

SEQ ID NO: 21 - protein, 12 aa, Arabidopsis thaliana
MGRLICTHFTLI

SEQ ID NO: 22 - protein, 13 aa, Arabidopsis thaliana
MLRSHETCTSRSI

SEQ ID NO: 23 - protein, 15 aa, Arabidopsis thaliana
MSAYQSSHKLSSSMS

SEQ ID NO: 24 - protein, 37 aa, Arabidopsis thaliana
MNKTQSHLLFSLSTCLNSYVNTKLPSILVNSKLSINT

SEQ ID NO: 25 - protein, 10 aa, Arabidopsis thaliana
MNHLQISLIS

SEQ ID NO: 26 - protein, 32 aa, Arabidopsis thaliana
MPKLICEHQTPIHSRQFKALYQYITSITLPMR

SEQ ID NO: 27 - protein, 15 aa, Arabidopsis thaliana
MGVWCSHMSLGMLIS

SEQ ID NO: 28 - protein, 16 aa, Arabidopsis thaliana
MGRGKVWVVEQSRRKI

SEQ ID NO: 29 - protein, 22 aa, Arabidopsis thaliana
MDGSLVFTYEFRHVDKLKSKWD

SEQ ID NO: 30 - protein, 137 aa, Arabidopsis thaliana
MVKWSGDVTTDCILCQGNLETREHLFFDCGYTSAVWAALVKGILKSRYTSNWTSIMDHLAHAQPHRVDHFLVRYAFQATLYTVWRERNGRRHGETLNTASQLVGWIDKQIRNQLSSIKLKGDRRYDDALQLWFSTRV

SEQ ID NO: 31 - protein, 41 aa, Arabidopsis thaliana
MLSHTAWTTSSFAMPSKRLYTLCGGKEMVDDTVRPSTRPLN

SEQ ID NO: 32 - protein, 14 aa, Arabidopsis thaliana
MVGRRYNGLHSLSR

SEQ ID NO: 33 - protein, 21 aa, Arabidopsis thaliana
MDRQANPKSAVLNQAQGRSTI

SEQ ID NO: 34 - protein, 28 aa, Arabidopsis thaliana
MSEVVHYRGPVGSISRFENSLYKRCPNC

SEQ ID NO: 35 - protein, 21 aa, Arabidopsis thaliana
MQRELKEFKLKIEFEKKETKL

SEQ ID NO: 36 - protein, 31 aa, Arabidopsis thaliana
MDNRDKSCFANKHAVAGKLIGFRTPAYSLKP

SEQ ID NO: 37 - protein, 28 aa, Arabidopsis thaliana
MCASRFKGVGGCSKTDQFTCNCMFICKT

SEQ ID NO: 38 - protein, 19 aa, Arabidopsis thaliana
MVTKETQAEGEKVCVIIIL

SEQ ID NO: 39 - protein, 16 aa, Arabidopsis thaliana
MLCCWDFDRSLPLILL

SEQ ID NO: 40 - protein, 59 aa, Arabidopsis thaliana
MGSLAEEGGDLLLPRGGVSGGAKTGEGLCSGDKGVVSVETSSRTSGIIPMLKHIIELRT

SEQ ID NO: 41 - protein, 40 aa, Arabidopsis thaliana
MVRCYRTSRTRCSIDRWGRVLLQLGDHIARPRIMIGEVKR

SEQ ID NO: 42 - protein, 23 aa, Arabidopsis thaliana
MWSPSWRSTLPQRSMLQRVRLVR

SEQ ID NO: 43 - protein, 161 aa, Arabidopsis thaliana
MPPQKENHRTLNKMKTNLFLFLIFSLLLSLSSAEQCGRQAGGALCPNGLCCSEFGWCGNTEPYCKQPGCQSQCTPGGTPPGPTGDLSGIISSSQFDDMLKHRNDAACPARGFYTYNAFITAAKSFPGFGTTGDTATRKKEVAAFFGQTSHETTGTQNSLTI

SEQ ID NO: 44 - protein, 63 aa, Arabidopsis thaliana
MICLSIGMMPLVLLEVSTLTTPLSPLQSPSPVLAPPETPPRGRRRSPPSSARLPMKLQVPKTH

SEQ ID NO: 45 - protein, 42 aa, Arabidopsis thaliana
MSTTKNFFRLYQNQHPNTSSSLLFYLALIDAKGLNRRESKTP

SEQ ID NO: 46 - protein, 12 aa, Arabidopsis thaliana
MLYAQALILCFI

SEQ ID NO: 47 - protein, 61 aa, Arabidopsis thaliana
MIMCSLFASVFDCFVPKSDSKISSTDESDLKVLSSKKPKSKSPRAPIMVSYFPAGSNLSRL

SEQ ID NO: 48 - protein, 17 aa, Arabidopsis thaliana
MKYWDVDFDINERSFWL

SEQ ID NO: 49 - protein, 10 aa, Arabidopsis thaliana
MLYKNSNDSM

SEQ ID NO: 50 - protein, 59 aa, Arabidopsis thaliana
MDRVKSNICSHIPLIGCVMDVLFLLFTSDSSSDAGKEIVDTGNLEAQKNFRQLQCIKLG

SEQ ID NO: 51 - protein, 21 aa, Arabidopsis thaliana
MTHPIRGICEQILDFTRSIKL

SEQ ID NO: 52 - protein, 14 aa, Arabidopsis thaliana
MPQLCLDLMKSLKL

SEQ ID NO: 53 - protein, 67 aa, Arabidopsis thaliana
MITVLTFTLNFQLSSLKGRRNYHGLLKGFMLRNTLSSFKPILYDPNNIGQLFAYFPLSVLEKENEVD

SEQ ID NO: 54 - protein, 18 aa, Arabidopsis thaliana
MIIPSSLKTRKLKVESKS

SEQ ID NO: 55 - protein, 11 aa, Arabidopsis thaliana
MSRRMILTQYW

SEQ ID NO: 56 - protein, 57 aa, Arabidopsis thaliana
MSLLLNLFMGLFLCGILKEVHKNEGEEEELVETELVEMELVETTSFYLITILKILFH

SEQ ID NO: 57 - protein, 10 aa, Arabidopsis thaliana
MKPYMINNFS

SEQ ID NO: 58 - protein, 36 aa, Arabidopsis thaliana
MSSPPIPSPLIPSPPIPPPPPRFYVPPSKSRRGKGP

SEQ ID NO: 59 - protein, 28 aa, Arabidopsis thaliana
MVANYLMKQYLQYCYQVKRCRLHQFHLH

SEQ ID NO: 60 - protein, 17 aa, Arabidopsis thaliana
MYLLQNPAEEKAHKQIE

SEQ ID NO: 61 - protein, 33 aa, Arabidopsis thaliana
MQENKARVTTEKRSVCRTPPINTENRFDCLMIC

SEQ ID NO: 62 - protein, 18 aa, Arabidopsis thaliana
MHLRKRRRLLSQKRTKVV

SEQ ID NO: 63 - protein, 22 aa, Arabidopsis thaliana
MARHSLLSPPYDSPLSLYIFSF

SEQ ID NO: 64 - protein, 15 aa, Arabidopsis thaliana
MTVLFHCTYSLFDDK

SEQ ID NO: 65 - protein, 14 aa, Arabidopsis thaliana
MLSRSSNNQICSPC

SEQ ID NO: 66 - protein, 11 aa, Arabidopsis thaliana
MKKICWASQLS

SEQ ID NO: 67 - protein, 36 aa, Arabidopsis thaliana
MDPAMEVDMVKGMEADTVVEIITVTHPKSFLLLIVS

SEQ ID NO: 68 - protein, 18 aa, Arabidopsis thaliana
MIIMCSMFCRYIYIHACI

SEQ ID NO: 69 - protein, 22 aa, Arabidopsis thaliana
MYIYVTTKHRTHYYHKFRIMKQ

SEQ ID NO: 70 - protein, 12 aa, Arabidopsis thaliana
MFCKFAIFHFYA

SEQ ID NO: 71 - protein, 11 aa, Arabidopsis thaliana
MEETLYTQHQK

SEQ ID NO: 72 - protein, 10 aa, Arabidopsis thaliana
MPENQHKSGE

SEQ ID NO: 73 - protein, 36 aa, Arabidopsis thaliana
MEDCELAEHDLVIETAVSSAGKLNPFLMFSYFWCCV

SEQ ID NO: 74 - protein, 19 aa, Arabidopsis thaliana
MRFLYNTLPGFQIDFFSSV

SEQ ID NO: 75 - protein, 33 aa, Arabidopsis thaliana
MCCQPLPLAIPLRTAVKPSRIKERQSKGVRSAT

SEQ ID NO: 76 - protein, 31 aa, Arabidopsis thaliana
MMFLQVNRSMLKKADSTIQRQSLANTETLAT

SEQ ID NO: 77 - protein, 45 aa, Arabidopsis thaliana
MISILSFRTSSSYYRQSFHKDWFLAAHVYQTIWELGILKLLGSQY

SEQ ID NO: 78 - protein, 31 aa, Arabidopsis thaliana
MGLLGEVVGNTLELGTILFLIISYELLLEFT

SEQ ID NO: 79 - protein, 12 aa, Arabidopsis thaliana
MSSSWNLLSFQS

SEQ ID NO: 80 - protein, 11 aa, Arabidopsis thaliana
MNELQRLYLKT

SEQ ID NO: 81 - protein, 30 aa, Arabidopsis thaliana
MIYESQDTTDKLIPEHMSNRLTMTGSKDIK

SEQ ID NO: 82 - protein, 24 aa, Arabidopsis thaliana
MIYESQDTTDKLISKHMSNRLKQT

SEQ ID NO: 83 - protein, 14 aa, Arabidopsis thaliana
MCLDINLSVVSWDS

SEQ ID NO: 84 - protein, 10 aa, Arabidopsis thaliana
MIVSKYLQPI

SEQ ID NO: 85 - protein, 26 aa, Arabidopsis thaliana
MSLDPVIVSLLLMCSGISLSVVSWDS

SEQ ID NO: 86 - protein, 81 aa, Arabidopsis thaliana
MRIMWKFIIVMLCFIALVGSRGTSATSRLRMKKEDIGRRFALQNKLQRGPVPPSQPSPCHNKLNPLSHSQVYSSHTYVTCP

SEQ ID NO: 87 - protein, 43 aa, Arabidopsis thaliana
MIKWLNNTKSCIIYVQKVLQFRLFRVWRSRIVDTYMYEKRDNK

SEQ ID NO: 88 - protein, 43 aa, Arabidopsis thaliana
MSKRYYNFDSFEFGGVESWIHICMRRETINKRMWLIKLQGQVT

SEQ ID NO: 89 - protein, 23 aa, Arabidopsis thaliana
MNFHMILMITRPCNAGMNGLVVD

SEQ ID NO: 90 - protein, 44 aa, Arabidopsis thaliana
MEKAVKEELDLVGAYSAKQTFYLYLPSSFSALTLPMFLLIPPKL

SEQ ID NO: 91 - protein, 12 aa, Arabidopsis thaliana
MRDQFNLQISAW

SEQ ID NO: 92 - protein, 35 aa, Arabidopsis thaliana
MNQIQLIYFNFLQPQCTIYLIFLFTKVFIYNTKNI

SEQ ID NO: 93 - protein, 28 aa, Arabidopsis thaliana
MGHHRILRKTHTSYIFRVIYKNFGEQKN

SEQ ID NO: 94 - protein, 26 aa, Arabidopsis thaliana
MIHHLYLARNPRISDTTSLITPILKC

SEQ ID NO: 95 - protein, 12 aa, Arabidopsis thaliana
MQNQIEKRCQFY

SEQ ID NO: 96 - protein, 28 aa, Arabidopsis thaliana
MNNELQQLYLKTKSKSHKQDIISFFLIQ

SEQ ID NO: 97 - protein, 60 aa, Arabidopsis thaliana
MSPRTQVTNTATGTQATVRIVDQCSNGGLDLEEGVFRQLDTNSQGNARGHLIVNYEFVNC

SEQ ID NO: 98 - protein, 19 aa, Arabidopsis thaliana
MLFSYTNLLNVCICPLERR

SEQ ID NO: 99 - protein, 27 aa, Arabidopsis thaliana
MCAYVPSNAGDEYCDRNASDCENRGSV

SEQ ID NO: 100 - protein, 19 aa, Arabidopsis thaliana
MSCSDENLLTLDGVFNCVL

SEQ ID NO: 101 - protein, 14 aa, Arabidopsis thaliana
MTISYIFFLLKTAI

SEQ ID NO: 102 - protein, 42 aa, Arabidopsis thaliana
MTADLGKSSSFSSRRPGSLDVLFRRESAHIGRRLQLCSMIRY

SEQ ID NO: 103 - protein, 11 aa, Arabidopsis thaliana
MCNRIFPDICP

SEQ ID NO: 104 - protein, 36 aa, Arabidopsis thaliana
MMMIFQDPLSSHHYIAVLRRKKIYDIVIVEEILVRS

SEQ ID NO: 105 - protein, 18 aa, Arabidopsis thaliana
MILSLSRRYLFDREREKY

SEQ ID NO: 106 - protein, 43 aa, Arabidopsis thaliana
MKKLRQRVAKKIKQDICGDNSTVFFFLVILTTFLILNYLINSL

SEQ ID NO: 107 - protein, 28 aa, Arabidopsis thaliana
MWSISPRKKKQYCCHHKCLVLFSLPLFA

SEQ ID NO: 108 - protein, 21 aa, Arabidopsis thaliana
MTWSNVIQLSLELKQPATSGT

SEQ ID NO: 109 - protein, 14 aa, Arabidopsis thaliana
MPLPVQALSLQVWD

SEQ ID NO: 110 - protein, 12 aa, Arabidopsis thaliana
MWCKHSSLLFLR

SEQ ID NO: 111 - protein, 19 aa, Arabidopsis thaliana
MDQSQTWRDKACTGKGIVD

SEQ ID NO: 112 - protein, 45 aa, Arabidopsis thaliana
MRNSQVPEVAGCFNSSESWMTFDQVICLDVANGNASSFFHHYCSR

SEQ ID NO: 113 - protein, 17 aa, Arabidopsis thaliana
MIFTLETGGRSVYTTSG

SEQ ID NO: 114 - protein, 41 aa, Arabidopsis thaliana
MGYGRSLIWLAEDTFGEISRRCVREVNDRYKQSFACLASDM

SEQ ID NO: 115 - protein, 28 aa, Arabidopsis thaliana
MTDINKVSPVWLLICRKLRTRKIVSSFS

SEQ ID NO: 116 - protein, 41 aa, Arabidopsis thaliana
MKLPNSDEFGQDLRKRRNNFSCSQLSTYQKPNRRNFVYIGH

SEQ ID NO: 117 - protein, 13 aa, Arabidopsis thaliana
MKFKYMNTKLKNE

SEQ ID NO: 118 - protein, 42 aa, Arabidopsis thaliana
MNSDKIYENEETIFLVLNFLHIRSQTGETLFISVIDFSDASP

SEQ ID NO: 119 - protein, 14 aa, Arabidopsis thaliana
MQQRVNCGHHLDLL

SEQ ID NO: 120 - protein, 29 aa, Arabidopsis thaliana
MVNHLQQIQMVTAIDSLLHRLYQGRQRLN

SEQ ID NO: 121 - protein, 10 aa, Arabidopsis thaliana
MKERSKTQLN

SEQ ID NO: 122 - protein, 15 aa, Arabidopsis thaliana
MGYRNRSYLVKSLPF

SEQ ID NO: 123 - protein, 19 aa, Arabidopsis thaliana
MRSRRIKKTRRIKRTRRIN

SEQ ID NO: 124 - protein, 24 aa, Arabidopsis thaliana
MFFEQRLGFHLLAGLVWLVNPSRF

SEQ ID NO: 125 - protein, 18 aa, Arabidopsis thaliana
MLSNDANRETGKSLYLKG

SEQ ID NO: 126 - protein, 26 aa, Arabidopsis thaliana
MMQTVKLESLYISRDKTPELALLVVL

SEQ ID NO: 127 - protein, 45 aa, Arabidopsis thaliana
MPITSSLPILGSEAGKDGQEEDNTAASGGIMAAGTPVTHPKGKPV

SEQ ID NO: 128 - protein, 17 aa, Arabidopsis thaliana
MSFLAENVESRFLAKKC

SEQ ID NO: 129 - protein, 10 aa, Arabidopsis thaliana
MLNCFLAGKY

SEQ ID NO: 130 - protein, 18 aa, Arabidopsis thaliana
MNVVFGGKMLNCVLAGKY

SEQ ID NO: 131 - protein, 18 aa, Arabidopsis thaliana
MLIVVFGEKMLNCVLAGK

SEQ ID NO: 132 - protein, 10 aa, Arabidopsis thaliana
MFNVVFGGKC

SEQ ID NO: 133 - protein, 10 aa, Arabidopsis thaliana
MLNCVLARKY

SEQ ID NO: 134 - protein, 10 aa, Arabidopsis thaliana
MLNCVLAGKC

SEQ ID NO: 135 - protein, 23 aa, Arabidopsis thaliana
MSLIIQICFSLSERHLVVAYIHF

SEQ ID NO: 136 - protein, 35 aa, Arabidopsis thaliana
MALNNYRLQIPLKHLPIPRHKLRNELNNTNLLLLE

SEQ ID NO: 137 - protein, 31 aa, Arabidopsis thaliana
MMIIHLQVTQHSEKDRLRKNHTYKLFCKNIF

SEQ ID NO: 138 - protein, 14 aa, Arabidopsis thaliana
MYDFFSAYLFHYVV

SEQ ID NO: 139 - protein, 36 aa, Arabidopsis thaliana
MICRLRRLGREWSLVKIDWLKLRMFLRAISNYFCRC

SEQ ID NO: 140 - protein, 15 aa, Arabidopsis thaliana
MIFSQPIFFTMLCNL

SEQ ID NO: 141 - protein, 20 aa, Arabidopsis thaliana
MVSCSNVRCGLSGCFGRKRT

SEQ ID NO: 142 - protein, 27 aa, Arabidopsis thaliana
MSRLSLCVFVLLCAFAAKAAAQSAPNV

SEQ ID NO: 143 - protein, 19 aa, Arabidopsis thaliana
MCVCVAMCVCGQSSRSIRT

SEQ ID NO: 144 - protein, 26 aa, Arabidopsis thaliana
MMIASQAVLHSQLQATWLQQSQLKQN

SEQ ID NO: 145 - protein, 32 aa, Arabidopsis thaliana
MGLDGVTNVDFDELGVDVSKLLLSLKWFWLLD

SEQ ID NO: 146 - protein, 15 aa, Arabidopsis thaliana
MWILMNWVLTCRNCC

SEQ ID NO: 147 - protein, 12 aa, Arabidopsis thaliana
MNGSRWSNECGF

SEQ ID NO: 148 - protein, 18 aa, Arabidopsis thaliana
MVLVARLRLRAQARSCWC

SEQ ID NO: 149 - protein, 18 aa, Arabidopsis thaliana
MHGTDVTFRKDARSGCDL

SEQ ID NO: 150 - protein, 68 aa, Arabidopsis thaliana
MDASRSITSLPGTLPKGHIRSLHPSERSHLFHASFGDATVRLYPLDLTPFGRDTSSPVSYQSTSSSVS

SEQ ID NO: 151 - protein, 56 aa, Arabidopsis thaliana
MLPEASHPFLAPSLKVTSAPCILPKGHICSMHLLAMQPFVCTRLTLHLLAETPLHL

SEQ ID NO: 152 - protein, 10 aa, Arabidopsis thaliana
MAELLQQVRY

SEQ ID NO: 153 - protein, 12 aa, Arabidopsis thaliana
MTLTCISQLLSS

SEQ ID NO: 154 - protein, 38 aa, Arabidopsis thaliana
MSVLVADLLEQFRHHLVELQISMLSYKLLMRFSLRILL

SEQ ID NO: 155 - protein, 25 aa, Arabidopsis thaliana
MILKCWSSRFLRVSPYQNAHSLSLG

SEQ ID NO: 156 - protein, 17 aa, Arabidopsis thaliana
MRILIWTHSQEPGTPAL

SEQ ID NO: 157 - protein, 49 aa, Arabidopsis thaliana
MAFFTNHFTIVLLYQFIFLIRTINFVIIMYEPKNPYSVYMKVSIWQLLI

SEQ ID NO: 158 - protein, 11 aa, Arabidopsis thaliana
MSTKVNGGGDE

SEQ ID NO: 159 - protein, 29 aa, Arabidopsis thaliana
MDMMVVNTEAVVFTDLVILNTEEAVVLRI

SEQ ID NO: 160 - protein, 11 aa, Arabidopsis thaliana
MNREEAVVLWI

SEQ ID NO: 161 - protein, 25 aa, Arabidopsis thaliana
MGIRYLNGPKRTRPTSCKPNFPSDK

SEQ ID NO: 162 - protein, 62 aa, Arabidopsis thaliana
MGQKEPGPPAVNLISLQTNEGRDGGGCCTEMTRGKRPSPVKERSTFAGFGREVRQRRRTRGS

SEQ ID NO: 163 - protein, 12 aa, Arabidopsis thaliana
MKVVMVAAVVRR

SEQ ID NO: 164 - protein, 11 aa, Arabidopsis thaliana
MVFLRAISNYL

SEQ ID NO: 165 - protein, 11 aa, Arabidopsis thaliana
MICRPRRQGRD

SEQ ID NO: 166 - protein, 14 aa, Arabidopsis thaliana
MNMLLKSQRYMHYP

SEQ ID NO: 167 - protein, 32 aa, Arabidopsis thaliana
MILCEYIYEHALKVTKIYALPVDAPVTVGFVF

SEQ ID NO: 168 - protein, 10 aa, Arabidopsis thaliana
MFLSVLEFGS

SEQ ID NO: 169 - protein, 20 aa, Arabidopsis thaliana
MYLWGMQEAMMNDIVPSMMQ

SEQ ID NO: 170 - protein, 12 aa, Arabidopsis thaliana
MERCTCGACKKR

SEQ ID NO: 171 - protein, 22 aa, Arabidopsis thaliana
MCSHWKREWRDVLVGHARSDDE

SEQ ID NO: 172 - protein, 28 aa, Arabidopsis thaliana
MNHLNRPKVLTQGPLSKIFNSSNRYSLL

SEQ ID NO: 173 - protein, 16 aa, Arabidopsis thaliana
MSGYSYDCTGLFPVLL

SEQ ID NO: 174 - protein, 33 aa, Arabidopsis thaliana
MIARVCSQSYCEEELPINIQRHVCWKVFAVQAA

SEQ ID NO: 175 - protein, 17 aa, Arabidopsis thaliana
MSTSSTWNHMQHAIKHN

SEQ ID NO: 176 - protein, 15 aa, Arabidopsis thaliana
MRKYNNIESITSSNQ

SEQ ID NO: 177 - protein, 10 aa, Arabidopsis thaliana
MEPHATRNQT

SEQ ID NO: 178 - protein, 25 aa, Arabidopsis thaliana
MFDCVLHVVPCTRRTHGNQLDCLFY

SEQ ID NO: 179 - protein, 48 aa, Arabidopsis thaliana
MWFHVLDVLMVTSLIVYFIDCLMLCFQYCYTSACCLLFWVLGWEIIVC

SEQ ID NO: 180 - protein, 17 aa, Arabidopsis thaliana
MQQRLMMNLLDQWELVA

SEQ ID NO: 181 - protein, 64 aa, Arabidopsis thaliana
MHKLTSLHNPRRNRSWRRRLWRRGLRIMDLRVSNSWGNGFWRRDISLRRHFRRSRPNKRRCNRG

SEQ ID NO: 182 - protein, 22 aa, Arabidopsis thaliana
MLSFGFRQDLFDTTRLSKNGKK

SEQ ID NO: 183 - protein, 40 aa, Arabidopsis thaliana
MPERLMGTDCKFVGNMSTLVQIQLGPIISCLHNHFFFLHK

SEQ ID NO: 184 - protein, 10 aa, Arabidopsis thaliana
MEKKVTKHKK

SEQ ID NO: 185 - protein, 20 aa, Arabidopsis thaliana
MRKALDITRKPLSWSTGHTK

SEQ ID NO: 186 - protein, 16 aa, Arabidopsis thaliana
MEKAEHLSSSAHEKSS

SEQ ID NO: 187 - protein, 16 aa, Arabidopsis thaliana
MIQKSQTRLVIKLYFV

SEQ ID NO: 188 - protein, 10 aa, Arabidopsis thaliana
MNPTIDPTTI

SEQ ID NO: 189 - protein, 24 aa, Arabidopsis thaliana
MDFGSYGFNLPVLVMSRSYGGGGG

SEQ ID NO: 190 - protein, 12 aa, Arabidopsis thaliana
MHSQYYSPNHGY

SEQ ID NO: 191 - protein, 15 aa, Arabidopsis thaliana
MATNKIRLGTKNQTE

SEQ ID NO: 192 - protein, 53 aa, Arabidopsis thaliana
MKAFHYYQPKEIRPLLDIRFMPERYEVGLIWKHWAIYFIPTILSGFWFLVLFY

SEQ ID NO: 193 - protein, 13 aa, Arabidopsis thaliana
MYSDEMLLILGGL

SEQ ID NO: 194 - protein, 20 aa, Arabidopsis thaliana
MADLSPNLLKQPKGLALSRF

SEQ ID NO: 195 - protein, 15 aa, Arabidopsis thaliana
MVLASSLLGARPFVG

SEQ ID NO: 196 - protein, 17 aa, Arabidopsis thaliana
MSRNLTHRPKRRENTRT

SEQ ID NO: 197 - protein, 23 aa, Arabidopsis thaliana
MDELQGGRRQAPCFQRSGGPNLI

SEQ ID NO: 198 - protein, 17 aa, Arabidopsis thaliana
MLSEKWRSESYLNCENN

SEQ ID NO: 199 - protein, 79 aa, Arabidopsis thaliana
MRNQSHVKTRHFAERFFNDSKIFFSFPDKISCFYEYPFLDSFRKLLFFVDLEICIDDLKSKVESLSLLLILFQFVLQLI

SEQ ID NO: 200 - protein, 20 aa, Arabidopsis thaliana
MGTRKNTRFYQGRRRKFWSR

SEQ ID NO: 201 - protein, 15 aa, Arabidopsis thaliana
MEYKCFNSDFTAVEK

SEQ ID NO: 202 - protein, 28 aa, Arabidopsis thaliana
MLMVKMDQKDPDTISMSLFSCFLTFPLQ

SEQ ID NO: 203 - protein, 28 aa, Arabidopsis thaliana
MAYDVRGNNVTTHVMGPFSGPSEVWLGP

SEQ ID NO: 204 - protein, 23 aa, Arabidopsis thaliana
MLWAHLVAQVKSGLALKSMEGLM

SEQ ID NO: 205 - protein, 36 aa, Arabidopsis thaliana
MPPPPKRQAVGPRVYAIAGEEDVDEDGADPIVGKSS

SEQ ID NO: 206 - protein, 20 aa, Arabidopsis thaliana
MLMRMAPTRLLVSLLKNLFK

SEQ ID NO: 207 - protein, 14 aa, Arabidopsis thaliana
MSIACACFWCSCLS

SEQ ID NO: 208 - protein, 21 aa, Arabidopsis thaliana
MAYTRGPTACRFGGGGIIPLF

SEQ ID NO: 209 - protein, 22 aa, Arabidopsis thaliana
MMPRRPTRRLYSSLKTPLRHDA

SEQ ID NO: 210 - protein, 59 aa, Arabidopsis thaliana
MRRLGSLDLLATGFFSDLEIRWCIALVLFFLLFVKPPRFFIVFARSWSLWCLLLLLFCT

SEQ ID NO: 211 - protein, 20 aa, Arabidopsis thaliana
MFLLLSCSIVLFQISIAHRL

SEQ ID NO: 212 - protein, 10 aa, Arabidopsis thaliana
MNVRIHPITN

SEQ ID NO: 213 - protein, 16 aa, Arabidopsis thaliana
MSGYIRSQTNTRAHEY

SEQ ID NO: 214 - protein, 13 aa, Arabidopsis thaliana
MFFLQLQLHRGLV

SEQ ID NO: 215 - protein, 26 aa, Arabidopsis thaliana
MKRTLKAQRMILKLLPVSKMHCLSNL

SEQ ID NO: 216 - protein, 23 aa, Arabidopsis thaliana
MSLVYWVALFHVHAVNNFHLPQP

SEQ ID NO: 217 - protein, 64 aa, Arabidopsis thaliana
MHMEEGHPIDQAHQIYQARQNDVQNPSALATTIYNRHFFGFSIVQLKLVPNCNMLSIGSMVFFL

SEQ ID NO: 218 - protein, 30 aa, Arabidopsis thaliana
MFFTLTIESAVCFINRRRGLGSKGDHRDQG

SEQ ID NO: 219 - protein, 14 aa, Arabidopsis thaliana
MPCCLAPSPDWTRH

SEQ ID NO: 220 - protein, 26 aa, Arabidopsis thaliana
MSGPVRRWSEATRHVRRDRSGNALFM

SEQ ID NO: 221 - protein, 22 aa, Arabidopsis thaliana
MEASGTKVPSESRNIALLLDRL

SEQ ID NO: 222 - protein, 24 aa, Arabidopsis thaliana
MSNPELISHTQQKELRWQRLHLVM

SEQ ID NO: 223 - protein, 24 aa, Arabidopsis thaliana
MIRCFCVTTKVLSFFRFKCCITLV

SEQ ID NO: 224 - protein, 12 aa, Arabidopsis thaliana
MFLCDYQSFELF

SEQ ID NO: 225 - protein, 14 aa, Arabidopsis thaliana
MLYHFGVGSWWFIR

SEQ ID NO: 226 - protein, 13 aa, Arabidopsis thaliana
MTRCSLCHRSSFC

SEQ ID NO: 227 - protein, 10 aa, Arabidopsis thaliana
MNPESSSAES

SEQ ID NO: 228 - protein, 13 aa, Arabidopsis thaliana
MRMRRGTRRRASR

SEQ ID NO: 229 - protein, 23 aa, Arabidopsis thaliana
MISDPSFAEALHKRRLSSAKRRW

SEQ ID NO: 230 - protein, 20 aa, Arabidopsis thaliana
MIVSSCAKPQRMRDQRSCVA

SEQ ID NO: 231 - protein, 21 aa, Arabidopsis thaliana
MGKPRIKSSTIPSHLLFLGLR

SEQ ID NO: 232 - protein, 42 aa, Arabidopsis thaliana
MLWPIPSRRHPRQTSKDPKEAKAIAKQTVMSQKLLGEGSSQF

SEQ ID NO: 233 - protein, 14 aa, Arabidopsis thaliana
MPSCLDRKPNLLKL

SEQ ID NO: 234 - protein, 16 aa, Arabidopsis thaliana
MYYPNIKPENNLNFVF

SEQ ID NO: 235 - protein, 23 aa, Arabidopsis thaliana
MNHDLYLLKYSVLCEVNIGPVLG

SEQ ID NO: 236 - protein, 12 aa, Arabidopsis thaliana
MSSVLILIAFQD

SEQ ID NO: 237 - protein, 16 aa, Arabidopsis thaliana
MYTLVLLNVKCRRCLY

SEQ ID NO: 238 - protein, 33 aa, Arabidopsis thaliana
MLNVVGAYINCFSRLENKVKIVFRFNVWVVHIY

SEQ ID NO: 239 - protein, 19 aa, Arabidopsis thaliana
MYTIMRFSYRISSTSSRFR

SEQ ID NO: 240 - protein, 18 aa, Arabidopsis thaliana
MILHLKVHLNLELVLEIR

SEQ ID NO: 241 - protein, 21 aa, Arabidopsis thaliana
MFRADELLEKDEVTSFVRRAS

SEQ ID NO: 242 - protein, 16 aa, Arabidopsis thaliana
MTKLCFELTSFLRRTR

SEQ ID NO: 243 - protein, 10 aa, Arabidopsis thaliana
MYRTLSTRSS

SEQ ID NO: 244 - protein, 11 aa, Arabidopsis thaliana
MIEARTLSGQA

SEQ ID NO: 245 - protein, 12 aa, Arabidopsis thaliana
MMTWSREYDTSV

SEQ ID NO: 246 - protein, 54 aa, Arabidopsis thaliana
MLPPSYFPLTRGVYLPRFLLDHLNTSVIVTHGRRCETHLILEEGQVCMPPPLLV

SEQ ID NO: 247 - protein, 27 aa, Arabidopsis thaliana
MISPLSELIASSFSLVRDSTFFSRTKS

SEQ ID NO: 248 - protein, 25 aa, Arabidopsis thaliana
MIRTGHASTVIFSFNPGGISSTLSS

SEQ ID NO: 249 - protein, 158 aa, Arabidopsis thaliana
MTYKGAGRFTSRKKLSPRYVGPYKVIKRVGAVAYKLDLPPKLNAFHNVFHVSQLRKCLSDQEESVEDIPPGLKENMTVEAWPVRIMDRMTKGTREKSRDLLKVLWNCGGREEYTWETENKMKANFPEWFKEMGKDQLDADSRTNPIQGGETCNARDPQ

SEQ ID NO: 250 - protein, 15 aa, Arabidopsis thaliana
MRFTTSSMCHNYGSV

SEQ ID NO: 251 - protein, 25 aa, Arabidopsis thaliana
MRIRGRIQFKGGRLVTPAILNRIVG

SEQ ID NO: 252 - protein, 30 aa, Arabidopsis thaliana
MAGTDHGPNDERDSGKIKGFVKSLVELWRP

SEQ ID NO: 253 - protein, 42 aa, Arabidopsis thaliana
MILFCLRLLKLSTAHSRLKRSASKRDAIFEYDRRLWNRGLPE

SEQ ID NO: 254 - protein, 14 aa, Arabidopsis thaliana
MLHLPKLSIGLLQV

SEQ ID NO: 255 - protein, 31 aa, Arabidopsis thaliana
MQFSNMIDDYGTEVYLNDHEESRRPHSSSLV

SEQ ID NO: 256 - protein, 50 aa, Arabidopsis thaliana
MQRRGWQQTNQMHLQQIHPSPHLLIPIILGEARCLVLVSVTLDALGLNKR

SEQ ID NO: 257 - protein, 19 aa, Arabidopsis thaliana
MTGISCDIWMTSFLQRTIG

SEQ ID NO: 258 - protein, 61 aa, Arabidopsis thaliana
MTSWVLIEVLISLRSNQPIVRCRNDVIQISQEIPVMIWPSFLTRLTSFSLIRSRAKPMTRL

SEQ ID NO: 259 - protein, 12 aa, Arabidopsis thaliana
MAMFNISEAHHR

SEQ ID NO: 260 - protein, 11 aa, Arabidopsis thaliana
MSSRYRRRSRS

SEQ ID NO: 261 - protein, 21 aa, Arabidopsis thaliana
MQPFVGTRSTLHVLAETPLHP

SEQ ID NO: 262 - protein, 26 aa, Arabidopsis thaliana
MQGMDVTFRKGERNGCDLSEGCKERM

SEQ ID NO: 263 - protein, 21 aa, Arabidopsis thaliana
MQKTDVTFWKDARNGCDLSEG

SEQ ID NO: 264 - protein, 20 aa, Arabidopsis thaliana
MLWEASIRPLYKGGRPRPLE

SEQ ID NO: 265 - protein, 12 aa, Arabidopsis thaliana
MTGIKRRRRLLF

SEQ ID NO: 266 - protein, 12 aa, Arabidopsis thaliana
MFIFDGLKFKKI

SEQ ID NO: 267 - protein, 19 aa, Arabidopsis thaliana
MKFMDLVDPILVHGFSAEI

SEQ ID NO: 268 - protein, 17 aa, Arabidopsis thaliana
MEALRPKSCSSTPSTFH

SEQ ID NO: 269 - protein, 29 aa, Arabidopsis thaliana
MHELILLYTKQKRPKTIKAPGIPAPTAIA

SEQ ID NO: 270 - protein, 19 aa, Arabidopsis thaliana
MGLSVVDLWGWDETTPFNA

SEQ ID NO: 271 - protein, 31 aa, Arabidopsis thaliana
MATTRLLTCARERRRILRPSKRDQRKNKRDT

SEQ ID NO: 272 - protein, 15 aa, Arabidopsis thaliana
MVISFAGYSPKESAR

SEQ ID NO: 273 - protein, 11 aa, Arabidopsis thaliana
MISPRVLGIHL

SEQ ID NO: 274 - protein, 14 aa, Arabidopsis thaliana
MDRSKPLRIAYYGK

SEQ ID NO: 275 - protein, 11 aa, Arabidopsis thaliana
MTFLIWTDRSP

SEQ ID NO: 276 - protein, 17 aa, Arabidopsis thaliana
MIVTRKLGIGSGSSYLP

SEQ ID NO: 277 - protein, 29 aa, Arabidopsis thaliana
MTAQRMSTIGMKWIAFSADLNRDLFFFPG

SEQ ID NO: 278 - protein, 10 aa, Arabidopsis thaliana
MLNVVLTEKC

SEQ ID NO: 279 - protein, 10 aa, Arabidopsis thaliana
MLNCVLAGKC

SEQ ID NO: 280 - protein, 27 aa, Arabidopsis thaliana
MLNCVLAENVELRFGGKMLNVVLAGKC

SEQ ID NO: 281 - protein, 28 aa, Arabidopsis thaliana
MLIVVFGGKMLNVFFGGKMLNCVLAGKC

SEQ ID NO: 282 - protein, 19 aa, Arabidopsis thaliana
MLNCVFDGKMLNCVLVEKC

SEQ ID NO: 283 - protein, 10 aa, Arabidopsis thaliana
MLNCVLAEKC

SEQ ID NO: 284 - protein, 34 aa, Arabidopsis thaliana
MSFWRENVECRFDGKMLNVVFGRKMLNCFLAGKC

SEQ ID NO: 285 - protein, 19 aa, Arabidopsis thaliana
MLIVVFGGKMLNVFFGGKM

SEQ ID NO: 286 - protein, 28 aa, Arabidopsis thaliana
MVVVSVRDSKEVHDGRVMVEAMISWLRV

SEQ ID NO: 287 - protein, 18 aa, Arabidopsis thaliana
MYWIIVGFLARFGDAVEC

SEQ ID NO: 288 - protein, 11 aa, Arabidopsis thaliana
MLMLLKHDLFL

SEQ ID NO: 289 - protein, 14 aa, Arabidopsis thaliana
MQKLLHGGLFGILS

SEQ ID NO: 290 - protein, 29 aa, Arabidopsis thaliana
MIFAMTKFQNGKKKKKQIPKWVELMGCDA

SEQ ID NO: 291 - protein, 14 aa, Arabidopsis thaliana
MEKKKKNKFQNGWN

SEQ ID NO: 292 - protein, 33 aa, Arabidopsis thaliana
MRASKSTKKVFYSIIELSFKGGQETLCCVRMSR

SEQ ID NO: 293 - protein, 46 aa, Arabidopsis thaliana
MLSCERVSRRRKSSILLSSCRSKAVKKPFVAFECPVEGSTISYCCC

SEQ ID NO: 294 - protein, 10 aa, Arabidopsis thaliana
MQFPVITQCY

SEQ ID NO: 295 - protein, 36 aa, Arabidopsis thaliana
MSTTKQSGVGRLMLLRFFSLNCFGCYVVGILIDLCH

SEQ ID NO: 296 - protein, 47 aa, Arabidopsis thaliana
MHRKKSIFHYQYCVKIMRLLILLQIYNYRSINGRDLSKSQQHNIQNS

SEQ ID NO: 297 - protein, 22 aa, Arabidopsis thaliana
MSYGLTQLILLNGLDQPITHLI

SEQ ID NO: 298 - protein, 30 aa, Arabidopsis thaliana
MSLKLNELWVDPTHFVKWVGSTHNSFNLMG

SEQ ID NO: 299 - protein, 13 aa, Arabidopsis thaliana
MVDTGPVRSVLYS

SEQ ID NO: 300 - protein, 23 aa, Arabidopsis thaliana
MLRFLWWILVLLGRYCTVDGAAT

SEQ ID NO: 301 - protein, 49 aa, Arabidopsis thaliana
MGLLPKCWTFTDWYQPLMGEIMLLLGLTFSWPIVQEKVITSSLRHRYRN

SEQ ID NO: 302 - protein, 14 aa, Arabidopsis thaliana
MSDPIACLECQHET

SEQ ID NO: 303 - protein, 13 aa, Arabidopsis thaliana
MKPNYDSGGEDLK

SEQ ID NO: 304 - protein, 33 aa, Arabidopsis thaliana
MAFKSRKIGKNKSNIKENKRKGKEKSNQWVASH

SEQ ID NO: 305 - protein, 15 aa, Arabidopsis thaliana
MPLGTMSFKSRGGSY

SEQ ID NO: 306 - protein, 20 aa, Arabidopsis thaliana
MNAGRDRYSVPLHRKSNNPS

SEQ ID NO: 307 - protein, 14 aa, Arabidopsis thaliana
MYLSLMILQVMQSY

SEQ ID NO: 308 - protein, 19 aa, Arabidopsis thaliana
MHEPVAVDGPLGLKPGLAL

SEQ ID NO: 309 - protein, 32 aa, Arabidopsis thaliana
MSLWRLMDHWASSLALLCRLCSIEPERPLMCI

SEQ ID NO: 310 - protein, 48 aa, Arabidopsis thaliana
MRLGESEVFNSAQQSAEIRLFRRMAFGSQNYSTDFFSHSSYNSRDANV

SEQ ID NO: 311 - protein, 21 aa, Arabidopsis thaliana
MAEIYQNPNNITSKTVKTKET

SEQ ID NO: 312 - protein, 30 aa, Arabidopsis thaliana
MIFTRLRLCYCNYYARKNLYCNFGYQMPFF

SEQ ID NO: 313 - protein, 25 aa, Arabidopsis thaliana
MSTNQEKAKPEGDSYCFNHSEKAQL

SEQ ID NO: 314 - protein, 16 aa, Arabidopsis thaliana
MVCATNGERRKRTICV

SEQ ID NO: 315 - protein, 23 aa, Arabidopsis thaliana
MREKICTVILATKCHSSKQSDLC

SEQ ID NO: 316 - protein, 27 aa, Arabidopsis thaliana
MATPFFPIDPLSFSQLTHPRETDFAES

SEQ ID NO: 317 - protein, 15 aa, Arabidopsis thaliana
MMISMILPWLLLFFQ

SEQ ID NO: 318 - protein, 22 aa, Arabidopsis thaliana
MYKMYACMDVCMNACMDVWMYA

SEQ ID NO: 319 - protein, 33 aa, Arabidopsis thaliana
MHRYFVLGCRFSGFVKLDVEEADQVLIVLCGLT

SEQ ID NO: 320 - protein, 15 aa, Arabidopsis thaliana
MLLYLVYQVGTGTYQ

SEQ ID NO: 321 - protein, 49 aa, Arabidopsis thaliana
MHVWMYGCMHECMYGCMHECMYGCMHECMYGCMHECTDILFWDADFPGS

SEQ ID NO: 322 - protein, 14 aa, Arabidopsis thaliana
MWKRLIKYSSCFVG

SEQ ID NO: 323 - protein, 63 aa, Arabidopsis thaliana
MYGCMHECMYGCMDVCMNACMDVCMNACMDVCMNACMDVCMNAPIFCFGMQIFRVRETGCGRG

SEQ ID NO: 324 - protein, 49 aa, Arabidopsis thaliana
MASSCQLLAINVGEGFAAVVAASFGRSRANSFNSEVVPDVRLLPPALGG

SEQ ID NO: 325 - protein, 20 aa, Arabidopsis thaliana
MRSVRFLDSILSYNNFVLSN

SEQ ID NO: 326 - protein, 15 aa, Arabidopsis thaliana
MFGLRLLRRNLLQCF

SEQ ID NO: 327 - protein, 13 aa, Arabidopsis thaliana
MQVRGELFNRCLV

SEQ ID NO: 328 - protein, 68 aa, Arabidopsis thaliana
MQIAKEYTCTPSHSFSLFICCMLRSKSFLRCKLEGNSSIDVLSRKSTEILFEISGNILKREHPLRSRR

SEQ ID NO: 329 - protein, 24 aa, Arabidopsis thaliana
MSCLEKALRYCLRYQETSSNVSIL

SEQ ID NO: 330 - protein, 20 aa, Arabidopsis thaliana
MLKDISMRQETRPLTKQNFT

SEQ ID NO: 331 - protein, 21 aa, Arabidopsis thaliana
MFMFEFCSLWMCFEFECIVFE

SEQ ID NO: 332 - protein, 15 aa, Arabidopsis thaliana
MQITYRDDTINMRIE

SEQ ID NO: 333 - protein, 72 aa, Arabidopsis thaliana
MSGQISLTLFSCLLCHLDRLFAFQEPSLGLFGLLTLSKNTPYLETDTFTDTKITQTQCTQTQNTSKDYKTQT

SEQ ID NO: 334 - protein, 49 aa, Arabidopsis thaliana
MLISLSICQVRYLSLYSHVYCVISIGYLHFKNRVLACLGYSHYPKTHLI

SEQ ID NO: 335 - protein, 15 aa, Arabidopsis thaliana
MNIGTLLLMFYVSKR

SEQ ID NO: 336 - protein, 14 aa, Arabidopsis thaliana
MNRLVFLAKEVNNH

SEQ ID NO: 337 - protein, 17 aa, Arabidopsis thaliana
MLANAEVSIYKSLSRTL

SEQ ID NO: 338 - protein, 17 aa, Arabidopsis thaliana
MFMSSGFIDSTKRVSSI

SEQ ID NO: 339 - protein, 15 aa, Arabidopsis thaliana
MYHKDSIFLLLLVVL

SEQ ID NO: 340 - protein, 77 aa, Arabidopsis thaliana
MKTGDAYLSSGMYIHEIEMKKFFNGYSLLWLRRLTGSYTRDTFSGVYKTRGHEHRLQCTTKILSFFFFLWFSEFLFL

SEQ ID NO: 341 - protein, 11 aa, Arabidopsis thaliana
MFKLWGFLTQG

SEQ ID NO: 342 - protein, 14 aa, Arabidopsis thaliana
MLIFPLECTSTRLK

SEQ ID NO: 343 - protein, 40 aa, Arabidopsis thaliana
MNIVFNVPQRFYLSSSSCGSLSFYFCKSTQCIYRVLDNDL

SEQ ID NO: 344 - protein, 17 aa, Arabidopsis thaliana
MVFIESIFKDWRAEEAS

SEQ ID NO: 345 - protein, 25 aa, Arabidopsis thaliana
MLTQILPKHLQFKTNSLTTRIRDTP

SEQ ID NO: 346 - protein, 78 aa, Arabidopsis thaliana
MMVLIIRFARHSYDALILFLMIYQYRVSISCVFLCFPELLWSISNSSSQTIGFELQMFWQNLGQHFLVKICFFSQHFS

SEQ ID NO: 347 - protein, 14 aa, Arabidopsis thaliana
MFFFHSLSSCDYRS

SEQ ID NO: 348 - protein, 40 aa, Arabidopsis thaliana
MVCFSFIHSLHVIIDHDGSNYPFCKTFIRRSYFIFNDLSV

SEQ ID NO: 349 - protein, 21 aa, Arabidopsis thaliana
MMTYLSFSEALVIYHPHIISS

SEQ ID NO: 350 - protein, 34 aa, Arabidopsis thaliana
MTKVRSISSLVMITTPKATSSIIPIRRRQLLVEI

SEQ ID NO: 351 - protein, 44 aa, Arabidopsis thaliana
MKKENGIGTQMKKIITSFHILKKMSRSQQERSHQVKSLLHDQLH

SEQ ID NO: 352 - protein, 10 aa, Arabidopsis thaliana
MWKEVIIFFI

SEQ ID NO: 353 - protein, 10 aa, Arabidopsis thaliana
MSMSNTSKDS

SEQ ID NO: 354 - protein, 26 aa, Arabidopsis thaliana
MRNTGLSSAPSFLWCFSGDTFCWRSI

SEQ ID NO: 355 - protein, 30 aa, Arabidopsis thaliana
MAFLQVFFSMASWKSMGSHSANKQNKVRFS

SEQ ID NO: 356 - protein, 19 aa, Arabidopsis thaliana
MKIYFSLLSSSLLRFSSGT

SEQ ID NO: 357 - protein, 19 aa, Arabidopsis thaliana
MLLAISRIVLFFLSATPFC

SEQ ID NO: 358 - protein, 11 aa, Arabidopsis thaliana
MLRRMILQRNR

SEQ ID NO: 359 - protein, 10 aa, Arabidopsis thaliana
MSRANISQSI

SEQ ID NO: 360 - protein, 10 aa, Arabidopsis thaliana
MFARLMIKVS

SEQ ID NO: 361 - protein, 13 aa, Arabidopsis thaliana
MSLSILRKLGPHR

SEQ ID NO: 362 - protein, 27 aa, Arabidopsis thaliana
MNHSNAPRICSPNLITISRKKAIIFFI

SEQ ID NO: 363 - protein, 13 aa, Arabidopsis thaliana
MKNCWKLPSTGLP

SEQ ID NO: 364 - protein, 15 aa, Arabidopsis thaliana
MSLHICSSKQSCPIQ

SEQ ID NO: 365 - protein, 21 aa, Arabidopsis thaliana
MTREQQPLYVLFPSMLACMLV

SEQ ID NO: 366 - protein, 49 aa, Arabidopsis thaliana
MKKIIAFFLDIVIKLGEQILGALLWFMEAQLMGASNNSSFLFKHQSVVF

SEQ ID NO: 367 - protein, 13 aa, Arabidopsis thaliana
MLVDLLLDWTGLF

SEQ ID NO: 368 - protein, 15 aa, Arabidopsis thaliana
MEGKRTYNGCCSRVI

SEQ ID NO: 369 - protein, 51 aa, Arabidopsis thaliana
MICHVIKLEKSVEGGKVTRAWVARKVLTPSRNGGKVVDSPLISAVGKVEKS

SEQ ID NO: 370 - protein, 79 aa, Arabidopsis thaliana
MVLKLGLLCTNLVPESRPDMVKVVQYLDRQVSLPDFSPDSPGIGIVTPVLVGGSSTVISNISSPVTEFITHSIQYGIGR

SEQ ID NO: 371 - protein, 18 aa, Arabidopsis thaliana
MNVVNKKLIWPSWSKAPD

SEQ ID NO: 372 - protein, 48 aa, Arabidopsis thaliana
MLTVGFEPTPFRTRTLIWRLGPTRPYQLFVNNVHLQASNFTSHRPIPY

SEQ ID NO: 373 - protein, 63 aa, Arabidopsis thaliana
MLEITVELPPTKTGVTIPIPGESGEKSGNETCLSRYCTTFTMSGLDSGTRFVQSNPNFNTISI

SEQ ID NO: 374 - protein, 14 aa, Arabidopsis thaliana
MALRIPKLYLVLPV

SEQ ID NO: 375 - protein, 16 aa, Arabidopsis thaliana
MRRIPEPVQITQGMQI

SEQ ID NO: 376 - protein, 31 aa, Arabidopsis thaliana
MDCFNANTNQRREATERNRTCKVTSGLSASQ

SEQ ID NO: 377 - protein, 10 aa, Arabidopsis thaliana
MCFRNPAVEI

SEQ ID NO: 378 - protein, 11 aa, Arabidopsis thaliana
MGNSCASETQL

SEQ ID NO: 379 - protein, 66 aa, Arabidopsis thaliana
MTVTKAVTLKPRRFMVNLKMVKPFHLWMQVVQGVRWTKKLGSVLKVLSKVHTLLKAVLALKGQRMQ

SEQ ID NO: 380 - protein, 15 aa, Arabidopsis thaliana
MLMVLDKFCSSSLRN

SEQ ID NO: 381 - protein, 29 aa, Arabidopsis thaliana
MSLGHSQRQSIHILLHLYCLHWLRYPRLD

SEQ ID NO: 382 - protein, 13 aa, Arabidopsis thaliana
MQQDVYALSLRVS

SEQ ID NO: 383 - protein, 33 aa, Arabidopsis thaliana
MMNSNFPFICFRPSDFHGRHCIQSVNYIKRLDQ

SEQ ID NO: 384 - protein, 21 aa, Arabidopsis thaliana
MYCPITIDQIHQTRNSIPHCC

SEQ ID NO: 385 - protein, 24 aa, Arabidopsis thaliana
MRRQIFSFLGLTTLSSNLLVCIAP

SEQ ID NO: 386 - protein, 18 aa, Arabidopsis thaliana
MGQYIPKGSKKESLSRES

SEQ ID NO: 387 - protein, 32 aa, Arabidopsis thaliana
MKIHDQNSPISTIDSVNEQLPFLITDSNPFAQ

SEQ ID NO: 388 - protein, 12 aa, Arabidopsis thaliana
MGDFNSAAVMIH

SEQ ID NO: 389 - protein, 19 aa, Arabidopsis thaliana
MVHGFTPHTRITGQKDLSQ

SEQ ID NO: 390 - protein, 14 aa, Arabidopsis thaliana
MDLLLILELLGKRI

SEQ ID NO: 391 - protein, 21 aa, Arabidopsis thaliana
MNFHGRFQLGGGDDSLTSFQS

SEQ ID NO: 392 - protein, 23 aa, Arabidopsis thaliana
MKKTLIIMPGKNQNQRKKEQREI

SEQ ID NO: 393 - protein, 16 aa, Arabidopsis thaliana
MIDSSPFSNYAIFNNV

SEQ ID NO: 394 - protein, 15 aa, Arabidopsis thaliana
MPHQTQREEQDLLYL

SEQ ID NO: 395 - protein, 46 aa, Arabidopsis thaliana
MRTMQRCHIKLREKNRISYTYRQGKYNRCSIYLFQKDTYSRNSCFQ

SEQ ID NO: 396 - protein, 10 aa, Arabidopsis thaliana
MLHLLYFPCL

SEQ ID NO: 397 - protein, 30 aa, Arabidopsis thaliana
MWHLCMVLISLRSIWNYVLSFLVKAINTSF

SEQ ID NO: 398 - protein, 32 aa, Arabidopsis thaliana
MSFLNRYLSEKGRCCICYIFPVYKYKRSCSSL

SEQ ID NO: 399 - protein, 63 aa, Arabidopsis thaliana
MLKRVGNNSKHASGNNISLIGETLPTTSLASFSCLLLVCSVAFFSGKKLRKCSCLPPSFLNRL

SEQ ID NO: 400 - protein, 18 aa, Arabidopsis thaliana
MLFPEACFELFPTRLSIT

SEQ ID NO: 401 - protein, 32 aa, Arabidopsis thaliana
MGEDTNISLAFCQKRMPQSIQVKDMKMMRVRL

SEQ ID NO: 402 - protein, 26 aa, Arabidopsis thaliana
MKKARLQSPLCSRVNNKIAAYQSHTV

SEQ ID NO: 403 - protein, 17 aa, Arabidopsis thaliana
MAYFRSASRNSPTFLNR

SEQ ID NO: 404 - protein, 10 aa, Arabidopsis thaliana
MHRLLLIHPN

SEQ ID NO: 405 - protein, 13 aa, Arabidopsis thaliana
MLGSFVKRYGNTP

SEQ ID NO: 406 - protein, 35 aa, Arabidopsis thaliana
MNKRRPPTYNKMERLMESKYMMIWILKMKMEVFKR

SEQ ID NO: 407 - protein, 31 aa, Arabidopsis thaliana
MVIGYLLNTSIFILSIHIIMYLDSINLSILL

SEQ ID NO: 408 - protein, 123 aa, Arabidopsis thaliana
MKKRRRRKKISSNKSSICKLQKKKTAKVTKEEVVVKYEDEVVVDMEMDVVGGHMKTTQTKEVKTHQEVVGKDTQNQDTINQVSNATIVGSLDIMLLNVKLLATKNLRRRPTTLKKKFKKKTCY

SEQ ID NO: 409 - protein, 32 aa, Arabidopsis thaliana
MNKKRIISGTSIVVQVITCAGEKVCSRSLMNR

SEQ ID NO: 410 - protein, 15 aa, Arabidopsis thaliana
MEIINLFPTFTIFRA

SEQ ID NO: 411 - protein, 24 aa, Arabidopsis thaliana
MTLHNVLRCVTKRSLGYGIFDSDI

SEQ ID NO: 412 - protein, 32 aa, Arabidopsis thaliana
MCVVQSSRNHLVKVITSFSLLMIFQEKHGYIF

SEQ ID NO: 413 - protein, 11 aa, Arabidopsis thaliana
MLMYRMRSGAN

SEQ ID NO: 414 - protein, 34 aa, Arabidopsis thaliana; identical to SEQ ID NO: 350

SEQ ID NO: 415 - protein, 49 aa, Arabidopsis thaliana
MKKENGIGTQMKKIITSFHILKKMSRSQQERSHQVKSLLHHQLHQQVLK

SEQ ID NO: 416 - protein, 17 aa, Arabidopsis thaliana
MPWMKRSNQYKRMTHGS

SEQ ID NO: 417 - protein, 12 aa, Arabidopsis thaliana
MLQLWEVWTLCF

SEQ ID NO: 418 - protein, 35 aa, Arabidopsis thaliana
MASSIRTSKFWRIGVAFKEGNGERATLYKSSKSSV

SEQ ID NO: 419 - protein, 40 aa, Arabidopsis thaliana
MSTWKAIQNELSKGVKFKSTKIVGANTYRCVWSNQAEITW

SEQ ID NO: 420 - protein, 18 aa, Arabidopsis thaliana
MGIFFERKIRGVRNFQKV

SEQ ID NO: 421 - protein, 19 aa, Arabidopsis thaliana
MGVNFTSKWTQDNWREVGV

SEQ ID NO: 422 - protein, 45 aa, Arabidopsis thaliana
MSFHLFGLCCLHVASNHVHFHIHHDLVLVLDHHLFFGNFGRFLLL

SEQ ID NO: 423 - protein, 10 aa, Arabidopsis thaliana
MWKEVIIFFI

SEQ ID NO: 424 - protein, 10 aa, Arabidopsis thaliana
MSMSNTSKDS

SEQ ID NO: 425 - protein, 26 aa, Arabidopsis thaliana
MRNTGLSSAPSFLWCFSGDTFCWRSI

SEQ ID NO: 426 - protein, 24 aa, Arabidopsis thaliana
MGFKLFENFEHLGFFFQKIYPCFS

SEQ ID NO: 427 - protein, 23 aa, Arabidopsis thaliana
MISILQSNENVSFTFYLHLRFIS

SEQ ID NO: 428 - protein, 13 aa, Arabidopsis thaliana
MILFLFIFLVASH

SEQ ID NO: 429 - protein, 12 aa, Arabidopsis thaliana
MSKLPTIVAFDT

SEQ ID NO: 430 - protein, 56 aa, Arabidopsis thaliana
MWPPTTSISISTTTSSSYLTTTSSLVTLAVFFFCNLHIEDLFDDIFFLLLLFFISL

SEQ ID NO: 431 - protein, 30 aa, Arabidopsis thaliana; identical to SEQ ID NO: 355

SEQ ID NO: 432 - protein, 19 aa, Arabidopsis thaliana
MKIYFSLLSSSLLRFSSGT

SEQ ID NO: 433 - protein, 19 aa, Arabidopsis thaliana
MLLAISRIVLFFLSATPFC

SEQ ID NO: 434 - protein, 67 aa, Arabidopsis thaliana
MPLSSQYLRNSLDVNSPPRSDLMVLITRPLSFSTWALNFLKISNTSDFSFKKYTHVFLEKSSIKRRK

SEQ ID NO: 435 - protein, 38 aa, Arabidopsis thaliana
MSFRMLRTNILFFDIGTLVMRLLSWSLIERLLSFNLIS

SEQ ID NO: 436 - protein, 37 aa, Arabidopsis thaliana
MRMFPLPFTSIFDSSPKATFPLTDSSSSANILFLPHM

SEQ ID NO: 437 - protein, 11 aa, Arabidopsis thaliana
MISSFFFFFSS

SEQ ID NO: 438 - protein, 51 aa, Arabidopsis thaliana
MSPFSFSRTTGAPSVQRIFNFPFRIKRRIKKGWSVKSPVPSRTKFRINKGS

SEQ ID NO: 439 - protein, 32 aa, Arabidopsis thaliana
MRTFLGTYDKCLAVSENDFSDNPTVCGAGCNG

SEQ ID NO: 440 - protein, 26 aa, Arabidopsis thaliana
MIKQIVISSVVHKMSRHCDPFLHHTR

SEQ ID NO: 441 - protein, 37 aa, Arabidopsis thaliana
MTERVQVVLCFTLETRLSHGCRRNNQLSLYPLVKRSM

SEQ ID NO: 442 - protein, 27 aa, Arabidopsis thaliana
MIEVNTLTHAITTLESVLAKRTCNWSM

SEQ ID NO: 443 - protein, 14 aa, Arabidopsis thaliana
MDVEETTNCHSIHL

SEQ ID NO: 444 - protein, 42 aa, Arabidopsis thaliana
MCMPCYLVKKPLEGVKLTTRGTNEDLCGQQVGNSFGEEPGLP

SEQ ID NO: 445 - protein, 55 aa, Arabidopsis thaliana
MCLHILQLHVLFANTLSNVVIACVNVFTSIMEDRVLRQSYCRLVVHKDLRWFLLW

SEQ ID NO: 446 - protein, 78 aa, Arabidopsis thaliana
MCLLRSWKTGFFAKAIADLLSTKIFVGSSCGKLNSFKRFLNQIAWHTHDVAATYSASQVDRVTIGCFFDIHVKAVSPM

SEQ ID NO: 447 - protein, 78 aa, Arabidopsis thaliana
MLDSECLLEIYSLCLSLLLLKELNLIVWRFMRKRSIRFSKRWISYLGRLVLVLMCGVVRVILMNSCVWQRIILMKDGN

SEQ ID NO: 448 - protein, 48 aa, Arabidopsis thaliana
MRWINHKEVQNSLFQFPSFINIMRCQTQEFIRITRTTPHINTNTNLPR

SEQ ID NO: 449 - protein, 25 aa, Arabidopsis thaliana
MLLFRINLHTIRFNSFKSNKLKQRL

SEQ ID NO: 450 - protein, 67 aa, Arabidopsis thaliana
MNLIVVYAAPSMSRRSGLWGELKEVVSGLVGLLIIGGDFITILRVDERMGGNGRLSPDFLAFGDWIN

SEQ ID NO: 451 - protein, 14 aa, Arabidopsis thaliana
MREWVEMGGFHQIF

SEQ ID NO: 452 - protein, 28 aa, Arabidopsis thaliana
MGRVEGSCIWFGRAVDNWRRLYHHLESG

SEQ ID NO: 453 - protein, 31 aa, Arabidopsis thaliana
MLLAFFTVVETSMICFVDQILLPHSLYDQRK

SEQ ID NO: 454 - protein, 10 aa, Arabidopsis thaliana
MIKESEILYP

SEQ ID NO: 455 - protein, 16 aa, Arabidopsis thaliana
MDNRMRLIMGTKFHFL

SEQ ID NO: 456 - protein, 13 aa, Arabidopsis thaliana
MNITGTEWITECD

SEQ ID NO: 457 - protein, 13 aa, Arabidopsis thaliana
MSEAKEFGQRNKS

SEQ ID NO: 458 - protein, 49 aa, Arabidopsis thaliana
MRALTIRLETNFVSSLSFLARFSSIWMRRDWTCVWTRTKSCIYFIAFIV

SEQ ID NO: 459 - protein, 10 aa, Arabidopsis thaliana
MYILKYGHFS

SEQ ID NO: 460 - protein, 27 aa, Arabidopsis thaliana
MTILSLDRSRRAGGSIRIVYGFAKYGS

SEQ ID NO: 461 - protein, 14 aa, Arabidopsis thaliana
MDIFYPIYKRYISD

SEQ ID NO: 462 - protein, 23 aa, Arabidopsis thaliana
MDISLDTIYLVMLKNLGYNVTIV

SEQ ID NO: 463 - protein, 24 aa, Arabidopsis thaliana
MNVLICFLFRCVCSFLVIYILQFS

SEQ ID NO: 464 - protein, 21 aa, Arabidopsis thaliana
MHGTFRFARSCLYPLQLDSTI

SEQ ID NO: 465 - protein, 51 aa, Arabidopsis thaliana
MGHFVLLDRACTHSNLTVLFNSRVFAFCFFSSERARGSIFNVYYVLKLYFI

SEQ ID NO: 466 - protein, 34 aa, Arabidopsis thaliana
MGSGASGPVRSSQSSQAGGRFNDADPIAIDYGKY

SEQ ID NO: 467 - protein, 46 aa, Arabidopsis thaliana
MKKQQKTIKTSLNLEKLKSKRNLIFSVIYGDRISIIKTSTSLATLR

SEQ ID NO: 468 - protein, 11 aa, Arabidopsis thaliana
MKTNKNTKYII

SEQ ID NO: 469 - protein, 42 aa, Arabidopsis thaliana
MIMCGLTLRVACTRYRAVIDCYHNKRGILRKSQEWRKLLRMS

SEQ ID NO: 470 - protein, 13 aa, Arabidopsis thaliana
MDVQLLHQQSYSS

SEQ ID NO: 471 - protein, 23 aa, Arabidopsis thaliana
MNAGRGRYTFLHTYDEYDCWWSS

SEQ ID NO: 472 - protein, 12 aa, Arabidopsis thaliana
MEKVFKCFERVF

SEQ ID NO: 473 - protein, 24 aa, Arabidopsis thaliana
MTLHNVLRCVTKRSLGYGIFDSDI

SEQ ID NO: 474 - protein, 35 aa, Arabidopsis thaliana
MASSIRTSKFWRIGVAFKEGNGERATLYKSSKARL

SEQ ID NO: 475 - protein, 73 aa, Arabidopsis thaliana
MSKNRMFVLNIRNDIAQCLKMCYKEESWLWHLRFGHLNFGGLELLSRKEMVRGLPCINHPKQGCEVVFKFKAK

SEQ ID NO: 476 - protein, 20 aa, Arabidopsis thaliana
MFPSMSQCSQRATMIIGVYE

SEQ ID NO: 477 - protein, 12 aa, Arabidopsis thaliana
MKVVFLKLKRMV

SEQ ID NO: 478 - protein, 25 aa, Arabidopsis thaliana
MKPFSTISHTSCAPRIAFIRRLQLS

SEQ ID NO: 479 - protein, 52 aa, Arabidopsis thaliana
MLLWPTYLQRPSRTYLHLILDRLDRELSCLSFLSLSNHPFEFEKDYLHSPVQ

SEQ ID NO: 480 - protein, 10 aa, Arabidopsis thaliana
MEGNIVTRHL

SEQ ID NO: 481 - protein, 12 aa, Arabidopsis thaliana
MEKVLKCFERVF

SEQ ID NO: 482 - protein, 22 aa, Arabidopsis thaliana
MCKVPTKTIIIKVSFFLYKKIK

SEQ ID NO: 483 - protein, 14 aa, Arabidopsis thaliana
MMMVLVGTLHITYL

SEQ ID NO: 484 - protein, 24 aa, Arabidopsis thaliana
MPAPIIPIDLCLLLIVFGFFSFLI

SEQ ID NO: 485 - protein, 76 aa, Arabidopsis thaliana
MKLKTTLLYFLIQKERNFDDDGFGGHFTHYLSLVCLVGIYLYNKCAFIHLYSGRDCWLLKQILTLVTSHACTHNTY

SEQ ID NO: 486 - protein, 33 aa, Arabidopsis thaliana
MISSFLIGLEKMARSLPLCTSRYRSHFFPSKRN

SEQ ID NO: 487 - protein, 11 aa, Arabidopsis thaliana
MRSIAGGAQRK

SEQ ID NO: 488 - protein, 14 aa, Arabidopsis thaliana
MNTAPDTLPQRVTV

SEQ ID NO: 489 - protein, 12 aa, Arabidopsis thaliana
MLSPLTLSLTQQ

SEQ ID NO: 490 - protein, 55 aa, Arabidopsis thaliana
MSSGSTCLLFGLPGGVSLVGGGVTETTPSTGGSSKIIIVIVVLGTGLMDSAYVKR

SEQ ID NO: 491 - protein, 11 aa, Arabidopsis thaliana
MNREPIVDLLG

SEQ ID NO: 492 - protein, 10 aa, Arabidopsis thaliana
MTTVNRDLDR

SEQ ID NO: 493 - protein, 23 aa, Arabidopsis thaliana
MISILQSNKNVSFTFYLHLRFIS

SEQ ID NO: 494 - protein, 13 aa, Arabidopsis thaliana
MILFLFIFLVASH

SEQ ID NO: 495 - protein, 35 aa, Arabidopsis thaliana
MFPLPFTSIFDSSPKATFPLTDSSSSANILFLPHM

SEQ ID NO: 496 - protein, 30 aa, Arabidopsis thaliana
MLLNVKLLATKHLRRRPTTLKKKFKKKTCY

SEQ ID NO: 497 - protein, 32 aa, Arabidopsis thaliana; identical to SEQ ID NO: 409

SEQ ID NO: 498 - protein, 34 aa, Arabidopsis thaliana
MMVHSTIQDNQRFCNIGSFRDLFSRSESKSMIVI

SEQ ID NO: 499 - protein, 39 aa, Arabidopsis thaliana
MNHHLQLRGEVILVEVVVLQGEEDAVVVVQCPLHMFRVQ

SEQ ID NO: 500 - protein, 28 aa, Arabidopsis thaliana
MGDLTESPRVGSLFSFFDFCPLFFVKNV

SEQ ID NO: 501 - protein, 16 aa, Arabidopsis thaliana
MRSLASLSKSNRVIHS

SEQ ID NO: 502 - protein, 32 aa, Arabidopsis thaliana
MKEINQTKMSFSSIYTFLYWGVMPLFASSLDF

SEQ ID NO: 503 - protein, 16 aa, Arabidopsis thaliana
MPCSVRNVRSIFLPVF

SEQ ID NO: 504 - protein, 18 aa, Arabidopsis thaliana
MSDQSFFQFFKWIYQVFI

SEQ ID NO: 505 - protein, 12 aa, Arabidopsis thaliana
MQCMPCGHHHLS

SEQ ID NO: 506 - protein, 14 aa, Arabidopsis thaliana
MWKIRRLWKTKRAI

SEQ ID NO: 507 - protein, 21 aa, Arabidopsis thaliana
MNLVYSFGSSVLKDSAYVLEM

SEQ ID NO: 508 - protein, 17 aa, Arabidopsis thaliana
MMSTRHALHSSRTDLLT

SEQ ID NO: 509 - protein, 42 aa, Arabidopsis thaliana
MFYIALLVFQSLRIFHMSETCLLLPMVRIRPAFALEPSAIGP

SEQ ID NO: 510 - protein, 10 aa, Arabidopsis thaliana
MHCIQVAQIC

SEQ ID NO: 511 - protein, 12 aa, Arabidopsis thaliana
METISTFFTDLQ

SEQ ID NO: 512 - protein, 18 aa, Arabidopsis thaliana
MVTKKKFLHEIYHWYWEE

SEQ ID NO: 513 - protein, 40 aa, Arabidopsis thaliana
MEDKLYFDLFIFDPLAQMFVLSRFDELPFVITWIMIDLEM

SEQ ID NO: 514 - protein, 44 aa, Arabidopsis thaliana
MSSILNSKRPKFYCKSVKNVEIVSIFTPPNTNGKFREEISFLSP

SEQ ID NO: 515 - protein, 91 aa, Arabidopsis thaliana
MLRSFPFSLLPIPMVNFVKKFLFCHHNTSKPSPRLFLCFGHIYISKSIMIHVMTNGNSSNRDSTNICARGSNINKSKYSLSSISKACERIP

SEQ ID NO: 516 - protein, 132 aa, Arabidopsis thaliana
MVMIFSSKMCLVDHHHQSTDLLLVIHVLHLLLLLTMMLCSRNPNPNQRRLCRFMTSLCMMMRMCLSLFLSSKSLPLLLSQLGLRMFSLLSHLLLRSTGNRTVLLSMISWETIWGRKGQIVTERRRVVPFLMI

SEQ ID NO: 517 - protein, 18 aa, Arabidopsis thaliana
MENVNHQKKIRTLVVVVH

SEQ ID NO: 518 - protein, 50 aa, Arabidopsis thaliana
MELPFSSRSLSAPFFPKLFPMRSSKGELFCFLCFVGEDEIEEKTFSNLAD

SEQ ID NO: 519 - protein, 39 aa, Arabidopsis thaliana
MCERTRTKQKKHLPTSWHDGEIFGSSFLGNNEDLDSPCI

SEQ ID NO: 520 - protein, 74 aa, Arabidopsis thaliana
MTERFLAAASSGITKISTVPASEEKLTIRMKSLSLEMVIRVFLFPGRKYLRVIASKPNPKFSASSDLKSVIEVF

SEQ ID NO: 521 - protein, 25 aa, Arabidopsis thaliana
MSIAKASPPEYDNDPSLIVTNSPPD

SEQ ID NO: 522 - protein, 21 aa, Arabidopsis thaliana
MDRCRILAAMLLQLTLIRILQ

SEQ ID NO: 523 - protein, 25 aa, Arabidopsis thaliana
MSVRRGVCYYQRWIVVVFWRRCFCN

SEQ ID NO: 524 - protein, 116 aa, Arabidopsis thaliana
MTTNKVIAICQSGGEFVTIKDGSLSYSGGDAFAIDIDQNTSMTDFKSELAENFGFGLEAMTLKYFLPGNKKTLITISKDKDFIRMVNFSSDAGTVEIFVIPEEAAAKNLSVMPASR

SEQ ID NO: 525 - protein, 20 aa, Arabidopsis thaliana
MVSKDQQMLFKEVENYICLT

SEQ ID NO: 526 - protein, 13 aa, Arabidopsis thaliana
MRDVNRLQLWQTP

SEQ ID NO: 527 - protein, 14 aa, Arabidopsis thaliana
MFELMSSIYSSPPP

SEQ ID NO: 528 - protein, 20 aa, Arabidopsis thaliana
MLIDCNSGRLHNHSGTWPSI

SEQ ID NO: 529 - protein, 20 aa, Arabidopsis thaliana
MDPGICLNLCQAYIVLHLLE

SEQ ID NO: 530 - protein, 10 aa, Arabidopsis thaliana
MLGDDMYLDR

SEQ ID NO: 531 - protein, 35 aa, Arabidopsis thaliana
MTCTLIGKNLRTSHQVLMHIDRAILRQCSGTLLDL

SEQ ID NO: 532 - protein, 11 aa, Arabidopsis thaliana
MANLRSKAKPI

SEQ ID NO: 533 - protein, 96 aa, Arabidopsis thaliana
MHFTPALKLATPSLVPSSSIGFTKTATASRAIVAEISPEGTRGTTMERKVSKAETATGSSVEESTEDIAAAKKAEGDGGRDSEVRIERIATAVATR

SEQ ID NO: 534 - protein, 15 aa, Arabidopsis thaliana
MGNYLGTFINGEFKK

SEQ ID NO: 535 - protein, 80 aa, Arabidopsis thaliana
MKLPSKMATLISPNSLWTVTASPQPLSTVISSPPPSLCALFSPQSLFHHLLQLSSQLLYLLSILQLKIPWPFLLCLLSFP

SEQ ID NO: 536 - protein, 23 aa, Arabidopsis thaliana
MIGNLFKLVLLYFLNSPLINVPR

SEQ ID NO: 537 - protein, 18 aa, Arabidopsis thaliana
MNIMKPFRRCRHSSCCKL

SEQ ID NO: 538 - protein, 13 aa, Arabidopsis thaliana
MPPLELLQVVSAI

SEQ ID NO: 539 - protein, 25 aa, Arabidopsis thaliana
MWLDCTYNLQQLEWRHLLKGFMMFM

SEQ ID NO: 540 - protein, 16 aa, Arabidopsis thaliana
MEDNILGICVSNETLF

SEQ ID NO: 541 - protein, 19 aa, Arabidopsis thaliana
MAPEFSMDPSRLLVTGFTS

SEQ ID NO: 542 - protein, 46 aa, Arabidopsis thaliana
MLSSMVVPVNGFSLNLSKPNLVVFNGSRVFYGSFSLTCYRFYFLII

SEQ ID NO: 543 - protein, 35 aa, Arabidopsis thaliana
MLFIYKFYWHMQIYVEVKRKTKQYAIVEMSFLGNG

SEQ ID NO: 544 - protein, 27 aa, Arabidopsis thaliana
MKCYLYINSIGTCKYTLKSKEKQNSTQ

SEQ ID NO: 545 - protein, 22 aa, Arabidopsis thaliana
MASIKNIGAKRKIEVKIFCLMT

SEQ ID NO: 546 - protein, 10 aa, Arabidopsis thaliana
MKSECVYMMS

SEQ ID NO: 547 - protein, 11 aa, Arabidopsis thaliana
MVITYTFFVPL

SEQ ID NO: 548 - protein, 48 aa, Arabidopsis thaliana
MAPVTVIKTSCIRILISFLTEEVTSFSFFASIACRRHIQHFISSCSSC

SEQ ID NO: 549 - protein, 10 aa, Arabidopsis thaliana
MYTHSDFISN

SEQ ID NO: 550 - protein, 57 aa, Arabidopsis thaliana
MSLEDPLKSPNKGEKAISRPFAIARRAARLYTAWLRYRSYVVIGLKSGIPAIQAYFF

SEQ ID NO: 551 - protein, 12 aa, Arabidopsis thaliana
MAGIPLLRPITT

SEQ ID NO: 552 - protein, 18 aa, Arabidopsis thaliana
MTSLAFSRSRIETPQVSR

SEQ ID NO: 553 - protein, 18 aa, Arabidopsis thaliana
MFLTSALGTLVMIFDVKC

SEQ ID NO: 554 - protein, 41 aa, Arabidopsis thaliana
MKQSCFKYYLMFSGLICRRGAYWLNRSKSTDVMTKLWCYTI

SEQ ID NO: 555 - protein, 10 aa, Arabidopsis thaliana
MSSRNTFISV

SEQ ID NO: 556 - protein, 25 aa, Arabidopsis thaliana
MKVFLEDMPQPSCCKKEKQILKSAR

SEQ ID NO: 557 - protein, 34 aa, Arabidopsis thaliana
MYCKQQEEKKTTLREMNWRVDVKSSQAVKKLRID

SEQ ID NO: 558 - protein, 103 aa, Arabidopsis thaliana
MTSSIKSKMGRVTSWKKSATSTTTRVAIKDTTSSKPTIPTTPTAAPTLLTLRIRCILHSNNKVRTNLLFPTTKVSFLNNNSREITSNHHHLGCTSAKPRSYCS

SEQ ID NO: 559 - protein, 11 aa, Arabidopsis thaliana
MHDSVSRILVV

SEQ ID NO: 560 - protein, 24 aa, Arabidopsis thaliana
MSSLCILGFQRRVNLRTFLNLRRA

SEQ ID NO: 561 - protein, 17 aa, Arabidopsis thaliana
MIGENQITSLDTLEHIF

SEQ ID NO: 562 - protein, 13 aa, Arabidopsis thaliana
MLYLKNISLSYLT

SEQ ID NO: 563 - protein, 32 aa, Arabidopsis thaliana
MSSVLQFHFYLPKLVREFFQNFAGLWLLSFRR

SEQ ID NO: 564 - protein, 22 aa, Arabidopsis thaliana
MCSRVSREVIWFSPIIYCYQGS

SEQ ID NO: 565 - protein, 33 aa, Arabidopsis thaliana
MECIPQNSHGVPLSLLTTQALHLTILWLVTGQT

SEQ ID NO: 566 - protein, 29 aa, Arabidopsis thaliana
MGSTSLLLAKGLGISTRTRILETSTLLTQ

SEQ ID NO: 567 - protein, 26 aa, Arabidopsis thaliana
MKMKRMAFCAKNASVLHHQLHTGVIR

SEQ ID NO: 568 - protein, 18 aa, Arabidopsis thaliana
MRLPRIVRLKESFCWHCE

SEQ ID NO: 569 - protein, 13 aa, Arabidopsis thaliana
MLVSCITNSTLVL

SEQ ID NO: 570 - protein, 12 aa, Arabidopsis thaliana
MGLHGNFGEYTP

SEQ ID NO: 571 - protein, 14 aa, Arabidopsis thaliana
MAHRLCSSLLGQQG

SEQ ID NO: 572 - protein, 25 aa, Arabidopsis thaliana
MGILGNTLHEIAQNSEIEGELLLAL

SEQ ID NO: 573 - protein, 22 aa, Arabidopsis thaliana
MEEAFSPLHLFPRICYTCLQQR

SEQ ID NO: 574 - protein, 18 aa, Arabidopsis thaliana
MILLSLTLLQTCIANAGK

SEQ ID NO: 575 - protein, 22 aa, Arabidopsis thaliana
MEHTNISFGKTRVFRGSLCDFP

SEQ ID NO: 576 - DNA, 30 bp, Arabidopsis thaliana
  source 1..30: /organism=“Arabidopsis thaliana” /mol_type=“unassigned DNA”
atgctatgtgtgcttcaaggtttaagggag

SEQ ID NO: 577 - DNA, 51 bp, Arabidopsis thaliana
  source 1..51: /organism=“Arabidopsis thaliana” /mol_type=“unassigned DNA”
atgataattattaataatgataattatttattattgttttataataataat

SEQ ID NO: 578 - DNA, 39 bp, Arabidopsis thaliana
  source 1..39: /organism=“Arabidopsis thaliana” /mol_type=“unassigned DNA”
atgataattatttattattgttttataataataattaat

SEQ ID NO: 579 - DNA, 33 bp, Arabidopsis thaliana
  source 1..33: /organism=“Arabidopsis thaliana” /mol_type=“unassigned DNA”
atgctcatgtaccggatgagaagcggagcaaac

SEQ ID NO: 580 - DNA, 54 bp, Arabidopsis thaliana
  source 1..54: /organism=“Arabidopsis thaliana” /mol_type=“unassigned DNA”
atgggcttaaacgaggatagtgtgtttcgtagcataaagccttttaaaagccca

SEQ ID NO: 581 - DNA, 117 bp, Arabidopsis thaliana
  source 1..117: /organism=“Arabidopsis thaliana” /mol_type=“unassigned DNA”
atgaatgtattagcaagagctccacggctccgtcatcagctccagaatctgacacaagatcggagaaaaactcaaatgcagatgaagggtcaacgagtcagaacgacgtcgctgcag

SEQ ID NO: 582 - DNA, 63 bp, Arabidopsis thaliana
  source 1..63: /organism=“Arabidopsis thaliana” /mol_type=“unassigned DNA”
atggcgagagaagaaaaagaacaatctgtgtatgatatttacacgtttgcgtctctgctattg

(19) United States
(12) Patent Application Publication
(10) Pub. No.: US 2013/0326728 A1
(43) Pub. Date: Dec. 5, 2013

(54) AGENTS FOR INCREASED RESISTANCE AGAINST OXIDATIVE STRESS CONDITIONS

(71) Applicant: Katholieke Universiteit Leuven, (BE)

(72) Inventors: Bruno Cammue, Alsemberg (BE); Delphine Carron, Wemmel (BE); David Cassiman, Korbeek-Lo (BE); Barbara De Coninck, Ninove (BE); Janick Mathys, Heverlee (BE); Pieter Spincemaille, Rotselaar (BE); Karin Thevissen, Bierbeek (BE)

(73) Assignee: KATHOLIEKE UNIVERSITEIT LEUVEN, Leuven (BE)

(21) Appl. No.: 13/992,289

(22) PCT Filed: Dec. 7, 2011
(86) PCT No.: PCT/BE11/00070

§ 371 Date: Jun. 7, 2013

(30) Foreign Application Priority Data
Dec. 8, 2010 (GB) 1020737.1

(51) Int. Cl. C07K 7/06; C12N 15/82; C12N 15/00; C12N 15/80

(52) U.S. Cl. CPC C07K 7/06; C12N 15/80; C12N 15/82; C12N 15/00
USPC 800/278; 435/468; 435/471; 435/419; 435/243; 435/320.1; 514/21.6; 530/328; 536/23.6; 800/298

(57) ABSTRACT

The present invention relates to methods and materials able to confer an increased tolerance or resistance to oxidative stress in cells or organisms. In particular, the present invention provides peptides possessing pharmacological or biotherapeutic activity and nucleic acids encoding said peptides which can be used to improve the tolerance of a microbial or eukaryotic cell to oxidative stress, to confer oxidative stress tolerance to an organism when transfected herein, or for the treatment and the prophylaxis of a wide range of oxidative stress-related pathologies in mammals, including humans, particularly mitochondrial dysfunction related disorders.

### FIELD OF THE INVENTION

The present invention relates to methods and materials able to confer an increased tolerance to oxidative stress in host cells and organisms and an increased cell survival in the presence of agents that induce mitochondrial dysfunction and/or oxidative stress. In particular, the present invention provides peptides and nucleic acids encoding said peptides to improve the tolerance of a cell, preferably an eukaryotic cell, to oxidative stress, to confer oxidative stress tolerance to an organism when transfected herein or treated therewith, or to prevent, ameliorate, treat or alleviate oxidative stress related disorders.

### BACKGROUND OF THE INVENTION

Oxidative stress or the induction of reactive oxygen species (ROS), such as e.g. superoxide, hydrogen peroxide and hydroxyl (.OH) free radicals, has been implicated in a variety of phenomena in many biological systems as diverse as the initiation of a fungal infection in plants to the onset or causative factor of apoptosis and various diseases, including cardiovascular disease, atherosclerosis, Parkinson's disease and Alzheimer disease [1-4]. ROS can be derived from endogenous sources via the metabolism of oxygen containing species, such as during aerobic respiration, or from exogenous sources such as toxins and environmental pollutants and stress conditions. ROS are associated with oxidative damage at the cellular level: they react with biological molecules which in its turn can lead to the destruction of cells and cellular components (e.g. mitochondria), causing cells to lose their structure and/or function, thus affecting cell metabolism and catabolism. The dominant role of the mitochondria in the cellular or aerobic respiration process makes them particularly vulnerable to ROS. Oxidative stress and the concomitant ROS generation may thus lead to mitochondrial dysfunction and subsequently trigger apoptosis pathways.

### Oxidative Stress Related Disorders

Oxidative stress is implicated in a variety of disease states, including Alzheimer's disease, Parkinson's disease, inflammatory diseases, neurodegenerative diseases, heart disease, HIV disease, chronic fatigue syndrome, hepatitis, cancer, autoimmune diseases cancer, and aging. For instance, a direct link between exposure to paraquat and the development of Parkinson's disease has been reported [5,6].

More in particular, as also described above, oxidative stress is implicated in mitochondrial dysfunction. Mitochondrial dysfunction has been established to contribute to the pathology of numerous diseases and is suspected in many more. In humans, many muscular and neurological disorders, various forms of cancer, diabetes, obesity, other disorders and ageing are associated with mitochondrial dysfunction (as discussed e.g. in Wallace, 2005, Annu Rev Genet. 39, 359-407, Modica-Napolitano, 2004, Mitochondrion 4, 755-62 or Orth, 2001, Am J Med Genet. 106, 27-36). A role for loss of mitochondrial function in normal aging has long been suspected. Most hypotheses focus on free radical damage to mitochondrial DNA. Mitochondrial dysfunction also plays a central role in the pathogenesis of several inborn errors of metabolism (e.g. Wilson's disease (WD) and inborn errors in respiratory chain complexes) but also in the frequent non-alcoholic fatty liver disease (NAFLD) or non-alcoholic steatohepatitis, which is the hepatic manifestation of the metabolic syndrome, and in other associated disorders as diabetes and obesity. In this respect, there is growing evidence that mitochondrial dysfunction, particularly respiratory chain deficiency, plays an role in the pathophysiology of NAFLD, which is linked to the generation of ROS by the damaged respiratory chain [10].

Wilson's disease (WD) is a rare ( 1/30-100.000) autosomal recessive inborn error of metabolism caused by mutations in ATP7b, an ATPase that is localized to the trans-Golgi and the mitochondria and essential for copper excretion from hepatocytes. Copper toxicity in WD is thought to arise from a direct effect of copper on mitochondria, ultimately amounting to tissue damage in liver and brain. The known mechanisms by which excess copper directly causes mitochondrial dysfunction and damage, are via (1) a deficiency in the mitochondrial respiratory chain, at the level of the copper-dependent complex IV (cytochrome C oxidase) and generation of ROS; (2) a cross-linking of mitochondrial membranous proteins, resulting in contraction of the mitochondrial membrane; and (3) an increase in sphingomyelinase activity, thereby changing the ceramide content of the membranes (mitochondrial membrane and cell membrane), leading to a pro-apoptotic phenotype. WD or mitochondrial copper toxicity in this context can therefore be considered as a model for inherited ( 1/5000 in the population) and acquired mitochondrial dysfunction disorders in general (e.g. non-alcoholic fatty liver disease and the metabolic syndrome). There hence remains a need for treatments of mitochondrial dysfunction related disorders, particularly by targeting the generation and accumulation of ROS.

### Oxidative Stress in Plants

Plants have developed a sophisticated regulatory system which involves both production and scavenging of ROS in cells. During normal growth and development, this pathway monitors the level of ROS produced by metabolism and controls the expression and activity of ROS scavenging pathways, which include enzymatic and non-enzymatic components (anti-oxidants). In general, plant development and yield depend on the ability of the plant to manage oxidative stress, whether it is via signaling or scavenging pathways. Consequently, improvements in a plant's ability to withstand oxidative stress, or to obtain a higher degree of cross-tolerance once oxidative stress has been experienced, has significant value in agriculture.

### Improving Tolerance Against Oxidative Stress

It is clear that an increased tolerance to oxidative stress in host cells and organisms is advantageous for many biological systems. Interestingly, overexpression of a peptide that was initially purified from the secretions of various oxidatively stressed cells from human neural cell lines increases their tolerance for oxidative stress [7]. Similarly, Arabidopsis thaliana peptides LEA5 and AtMtATP6 both lead to increased oxidative stress tolerance when overexpressed in yeast [8, 9]. The late abundance protein LEA5 (At4g02380) is a 97 amino acids peptide that was isolated by screening an A. thaliana cDNA library for clones which enabled growth of the yeast Δyap1 mutant on toxic concentrations of H₂O₂. The 55 amino acids peptide AtMtATP6 (At3g46430) is a component of the mitochondrial F₁F₀-ATPase an enzyme that is involved in oxidative respiration. In A. thaliana suspension cultured cells, AtMtATP6 expression was induced upon osmotic, cold and oxidative stress, the latter caused by H₂O₂. Expression of AtMtATP6 improved the tolerance of wild-type yeast to various abiotic stresses like H₂O₂ and paraquat.

Based on these findings, the identification and characterization of novel Oxidative Stress Induced Peptides (OSIPs) could reveal novel pathways involved in governing tolerance/resistance of organisms against oxidative stress and can be used in novel strategies to protect an organism against (oxidative) stress and/or to improve the stress tolerance of said organism. Such knowledge on anti-oxidative stress peptides can have broad implications for understanding and treatment of diseases that are characterized by the induction of ROS, in particular mitochondrial dysfunction disorders, or can be used for improving stress tolerance in crops.

### SUMMARY OF THE INVENTION

The present invention relates to the identification of 176 oxidative stress induced peptides (OSIPs) encoding regions (Table 2), which correspond to 575 OSIP peptides in the different reading frames (Table 1, SEQ ID No. 1 to SEQ ID No. 575), which may be useful as agents to increase oxidative stress tolerance in cells or organisms and to increase cell survival in the presence of substances that induce mitochondrial dysfunction and/or oxidative stress. Preferred OSIP peptides that confer oxidative stress tolerance and resistance to a cell or organism comprise peptides with amino acid sequence selected from SEQ ID No. 1 to SEQ ID No. 7. Thus, the present invention relates to compounds of a peptidic nature possessing pharmacological or biotherapeutic activity, and to the nucleic acids encoding said peptides, which can be used for the treatment and the prophylaxis of a wide range of oxidative stress-induced pathologies in mammals, including humans and/or as oxidative stress tolerance increasing agents in a wide variety of cells and organisms, including yeast, fungi and plants.

A first embodiment of the present invention provides an isolated peptide comprising an amino acid sequence with 70% or 80%, more preferably at least 90%, more preferably at least 95%, 97%, 99% or 100% sequence identity to an amino acid sequence selected from SEQ ID No. 1 to SEQ ID No. 575; preferably selected from SEQ ID No. 1, SEQ ID No. 2, SEQ ID No. 3, SEQ ID No. 4, SEQ ID No. 5, SEQ ID No. 6 or SEQ ID No. 7, more preferably to amino acid sequence SEQ ID No. 1, wherein the isolated peptide increases the oxidative stress tolerance of a cell or organism under oxidative stress conditions, or stated differently, wherein the isolated peptide increases viability of a cell or organism under oxidative stress conditions and/or in the presence of substances that induce mitochondrial dysfunction.

Another embodiment of the present invention relates to an isolated nucleic acid encoding for a peptide comprising an amino acid sequence with 70% or 80%, more preferably at least 90%, more preferably at least 95%, 97%, 99% or 100% sequence identity to an amino acid sequence selected from SEQ ID No. 1 to SEQ ID No. 575; preferably selected from SEQ ID No. 1, SEQ ID No. 2, SEQ ID No. 3, SEQ ID No. 4, SEQ ID No. 5, SEQ ID No. 6 or SEQ ID No. 7, more preferably to amino acid sequence SEQ ID No. 1, wherein the isolated peptide increases the oxidative stress tolerance of a cell or organism under oxidative stress conditions. Preferably, said nucleic acid comprises a nucleotide sequence with at least 70% or 80%, more preferably at least 90%, more preferably at least 95%, 97%, 99% or 100% sequence identity to a nucleotide sequence selected from SEQ ID No. 576, SEQ ID No. 577, SEQ ID No. 578, SEQ ID No. 579, SEQ ID No. 580, SEQ ID No. 581 or SEQ ID No. 582, preferably to nucleotide sequence SEQ ID No. 576.

Another embodiment of the invention provides a genetic construct comprising the following operably linked DNA elements: (a) a nucleic acid encoding for a peptide comprising an amino acid sequence with 70% or 80%, more preferably at least 90%, more preferably at least 95%, 97%, 99% or 100% sequence identity to an amino acid sequence selected from SEQ ID No. 1 to SEQ ID No. 575; preferably selected from SEQ ID No. 1, SEQ ID No. 2, SEQ ID No. 3, SEQ ID No. 4, SEQ ID No. 5, SEQ ID No. 6 or SEQ ID No. 7, more preferably to amino acid sequence SEQ ID No. 1, or a nucleic acid comprising a nucleotide sequence with at least 70% or 80%, more preferably at least 90%, more preferably at least 95%, 97%, 99% or 100% sequence identity to a nucleotide sequence selected from SEQ ID No. 576, SEQ ID No. 577, SEQ ID No. 578, SEQ ID No. 579, SEQ ID No. 580, SEQ ID No. 581 or SEQ ID No. 582, respectively, preferably to nucleotide sequence SE SEQ ID No. 576; (b) one or more control sequences capable of driving expression of said nucleic acid, and (c) a 3′ end region comprising a transcription termination sequence.

In another embodiment, the present invention provides a method for increasing the oxidative stress tolerance of a cell or organism, preferably an eukaryotic cell or organism, or modulating the oxidative stress level in a cell or organism, preferably an eukaryotic cell or organism, comprising contacting or transfecting said cell or organism with (i) a peptide comprising an amino acid sequence with 70% or 80%, more preferably at least 90%, more preferably at least 95%, 97%, 99% or 100% sequence identity to an amino acid sequence selected from SEQ ID No. 1 to SEQ ID No. 575; preferably selected from SEQ ID No. 1, SEQ ID No. 2, SEQ ID No. 3, SEQ ID No. 4, SEQ ID No. 5, SEQ ID No. 6 or SEQ ID No. 7, more preferably to amino acid sequence SEQ ID No. 1; (ii) a peptidomimetic of (i); (iii) a nucleic acid encoding for (i); or (iv) a nucleic acid comprising a nucleotide sequence with at least 70% or 80%, more preferably at least 90%, more preferably at least 95%, 97%, 99% or 100% sequence identity to a nucleotide sequence selected from SEQ ID No. 576, SEQ ID No. 577, SEQ ID No. 578, SEQ ID No. 579, SEQ ID No. 580, SEQ ID No. 581 or SEQ ID No. 582, respectively, preferably to nucleotide sequence SEQ ID No. 576.

Preferably, said method includes contacting the cell with a peptide of the present invention, a peptidomimetic thereof or a nucleic acid encoding therefor, under conditions effective to increase cell survival in the presence of agents that induce mitochondrial dysfunction and/or oxidative stress (relative to the same cell under the same conditions without the oxidative stress tolerance inducing agent). The cell can be in a cell culture, a tissue, an organ, or an organism. Hence, the method can be carried out in vitro or in vivo. The cell can be a plant cell, a microbial cell, a fungal cell, a yeast cell, a mammalian cell, or a human cell.

In yet another preferred embodiment, said method for increasing the survival in the presence of oxidative stress-inducing agents or agents that induce mitochondrial dysfunction and/or for increasing the oxidative stress tolerance of a cell or non-human organism, preferably an eukaryotic cell or eukaryotic, non-human organism, comprising introducing and expressing into said cell or non-human organism (i) a nucleic acid encoding for a peptide comprising an amino acid sequence with 70% or 80%, more preferably at least 90%, more preferably at least 95%, 97%, 99% or 100% sequence identity to an amino acid sequence selected from SEQ ID No. 1 to SEQ ID No. 575; preferably selected from SEQ ID No. 1, SEQ ID No. 2, SEQ ID No. 3, SEQ ID No. 4, SEQ ID No. 5, SEQ ID No. 6, or SEQ ID No. 7, more preferably to amino acid sequence SEQ ID No. 1, or (ii) a nucleic acid comprising a nucleotide sequence with at least 70% or 80%, more preferably at least 90%, more preferably at least 95%, 97%, 99% or 100% sequence identity to a nucleotide sequence selected from SEQ ID No. 576, SEQ ID No. 577, SEQ ID No. 578, SEQ ID No. 579, SEQ ID No. 580, SEQ ID No. 581 or SEQ ID No. 582, preferably to nucleotide sequence SEQ ID No. 576. The cell can be a plant cell, a microbial cell, a fungal cell, a yeast cell, a mammalian cell, or a human cell. The cell can be in a cell culture, a tissue, an organ, or any non-human organism. Preferably, said nucleic acid is incorporated in the genetic construct according to another embodiment of the present invention. Preferably, said nucleic acid capable of encoding an oxidative stress tolerance increasing peptide is operably linked to a promoter that drives expression of a coding sequence in said cell. Preferably, said nucleic acid and/or chimeric genetic construct is stably incorporated in the genome of said cell.

Yet another embodiment of the present invention relates to a transgenic cell or non-human organism having an increased survival in oxidative stress conditions and/or having an increased tolerance to oxidative stress relative to the corresponding wild-type cell or non-human organism obtainable by the above method according to another embodiment of the present invention. Thus, the present invention also provides a transgenic cell or non-human organism having increased tolerance to oxidative stress relative to the corresponding wild-type cell or non-human organism wherein said transgenic cell or non-human organism is transfected with and expresses the nucleic acid or the genetic construct according to other embodiments of the present invention. Preferably, said transgenic cell or non-human organism is a plant cell or plant or part thereof, or a microbial eukaryotic cell or organism, such as a fungi/fungal cell or yeast. Thus, the present invention also provides transgenic plants and plant cells having increased levels of tolerance to oxidative stress, nucleic acids according to the present invention to generate transgenic plants and plant cells having increased levels of tolerance to oxidative stress, and methods for making plants and plant cells having increased levels of tolerance to oxidative stress. In another embodiment, the present invention also relates to harvestable parts, including seeds, of said transgenic plant.

Yet another embodiment of the present invention relates to a peptide of the present invention comprising an amino acid sequence with 70% or 80%, more preferably at least 90%, more preferably at least 95%, 97%, 99% or 100% sequence identity to an amino acid sequence selected from SEQ ID No. 1 to SEQ ID No. 575; preferably selected from SEQ ID No. 1, SEQ ID No. 2, SEQ ID No. 3, SEQ ID No. 4, SEQ ID No. 5, SEQ ID No. 6, or SEQ ID No. 7, more preferably to amino acid sequence SEQ ID No. 1, for use in the treatment or prevention of a mammal or a human having an oxidative stress related disorder. Preferably, said oxidative stress related disorder is a mitochondrial dysfunction related disorder.

Yet another embodiment of the present invention relates to a pharmaceutical composition comprising (i) a peptide comprising an amino acid sequence with 70% or 80%, more preferably at least 90%, more preferably at least 95%, 97%, 99% or 100% sequence identity to an amino acid sequence selected from SEQ ID No. 1 to SEQ ID No. 575; preferably selected from SEQ ID No. 1, SEQ ID No. 2, SEQ ID No. 3, SEQ ID No. 4, SEQ ID No. 5, SEQ ID No. 6, or SEQ ID No. 7, more preferably to amino acid sequence SEQ ID No. 1, or a peptidomimetic thereof, or a nucleic acid encoding therefor, and (ii) one or more pharmaceutically acceptable compounds, carriers and/or adjuvants.

Thus, the present invention also relates to the use of an oxidative stress tolerance inducing peptide of the present invention for modulating or controlling the oxidative stress level and/or mitochondrial dysfunction in a patient or for the prevention or treatment of oxidative stress related disorders, such as mitochondrial dysfunction related disorders, in an organism. In a particular embodiment, the present invention also relates to the use of said peptides or nucleic acids according to the present invention for the manufacture of a medicament for the prevention and/or treatment of (oxidative) stress related disorders, in particular mitochondrial dysfunction disorders, and to the use of said peptides or nucleic acids for the screening of materials for their therapeutic activity. Said method for modulating or controlling the oxidative stress level and/or mitochondrial dysfunction in a patient or for the prevention or treatment of oxidative stress related disorders in an organism includes administering to the patient or organism a therapeutically effective amount of a peptide of the present invention, a peptidomimetic thereof or a composition comprising said peptide or peptidomimetic, under conditions effective to decrease or prevent an increase in the level of at least one ROS species in the patient, and/or effective to decrease or prevent an increase in the level of mitochondrial dysfunction in the patient (relative to the same conditions without the oxidative stress tolerance inducing agent).

### DETAILED DESCRIPTION

### Legends of the Figures

FIG. 1 presents the different open reading frames, deduced from the 176 selected OSIP encoding regions, ordered based on their induction level.

FIG. 2 represents the results from the halo-test to assay the oxidative stress tolerance of the yeast in the presence & absence of OSIPs.

FIG. 3 shows the effect of exogenously added OSIP108 on (A) peroxide-induced ROS levels in yeast (square: in the absence of OSIP108, triangle: in the presence of OSIP108) (B) the survival rate of peroxide-treated yeast cells (black bar: in the absence of OSIP108, white bar: in the presence of OSIP108).

FIG. 4 shows the effect of OSIP108 on yeast growth inhibition induced by apoptosis-inducing compounds. Yeast growth was monitored upon treatment with different apoptosis-inducing compounds in the presence (black bars) or absence (white bars) of 200 μM OSIP108. Percentage growth was calculated as the ratio of the OD600 of the treated culture over an unstressed yeast culture after 10 h (A) or 48 h (B) of incubation.

FIG. 5 shows that OSIP108 significantly enhances copper resistance of human hepatoma cell line HepG2. The mean and SE of three experiments is shown. No: viability of cells without addition of peptide; OSIP: OSIP108 was added simultaneously added to cells together with Cu; OSIP pre, cells were preincubated with OSIP108 for 12 h prior to addition of Cu.

FIG. 6 shows that OSIP108 significantly enhances copper resistance of cells expressing wild-type hATP7B (A) and cells expressing the hATP7B H1069Q mutant frequently found in patients having WD (B). No: viability of cells without addition of peptide; OSIP: OSIP108 was added simultaneously added to cells together with Cu; and OSIP pre: cells were preincubated with the peptide for 12 h prior to addition of Cu.

### DESCRIPTION

Tiling array technology was used for the identification of novel—previously unannotated—peptide-encoding genes, particularly for the identification of specific peptide-encoding genes that were upregulated in A. thaliana during oxidative stress induced by the herbicide paraquat. This way, 176 potential ‘Oxidative Stress Induced Peptides’ (OSIPs) encoding regions were identified (listed in Table 2) which correspond to 575 OSIPs in the different open reading frames of said OSIP encoding regions (Table 1). The ability of said peptides to increase the oxidative stress resistance and/or survival in oxidative stress conditions of a host cell or organism, preferably an eukaryotic cell or organism, was evaluated in several biological systems, including overexpression in yeast and exogenous application of an OSIP peptide to yeast and other cell cultures and plants.

The scope of the applicability of the present invention will become apparent from the detailed description and drawings provided below. However, it should be understood that the detailed description and specific examples, while indicating preferred embodiments of the present invention, are given by way of illustration only since various changes and modifications within the spirit and scope of the invention will become apparent to those skilled in the art from this detailed description. Unless otherwise defined, all technical and scientific terms used herein have the same meaning as commonly understood by one of ordinary skill in the art to which this invention belongs.

Reference throughout this specification to “one embodiment” or “an embodiment” means that a particular feature, structure or characteristic described in connection with the embodiment is included in at least one embodiment of the present invention. Thus, appearances, of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments. Where an indefinite or definite article is used when referring to a singular noun e.g. “a” or “an”, “the”, this includes a plural of that noun unless something else is specifically stated.

Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects.

The phrase “oxidative stress conditions” as used herein, refers to conditions that results in oxidative stress and elevate the ROS level beyond the normal level, resulting in e.g. destruction of cells and cellular components (e.g. mitochondria), causing cells to lose their structure and/or function, and/or cell death. Particular oxidative stress conditions are those that result in or are related with mitochondrial dysfunction.

As used herein the phrase “oxidative stress” refers to an undesirable imbalance where in general oxidants outnumber antioxidants. This situation can particularly arise if the rate of ROS production overwhelms existing antioxidant defenses. In such circumstances, a series of cellular responses (e.g. mitochondrial dysfunction and the subsequent impaired respiratory chain and cellular respiration) can occur that can lead to an even greater increase in ROS production. Excessive ROS production and its otherwise ineffective regulation can be detrimental to cells and tissues, inducing cellular damage that ultimately can lead to cell death (apoptosis). Oxidative stress-associated damage also can cause undesirable changes to the structural and functional integrities of cells that can lead to the propagation of cells instead of apoptosis. Additionally, oxidatively-damaged cellular macromolecules can trigger immune responses that can lead to disease. See generally, D. G. Lindsay et al. (2002) Mol. Aspects of Med. 23:1-38. In the case of plants, oxidative stress occurs e.g. in situations of ozone stress, in cases of necrosis as a result of pathogen infection or wounding, in cases of senescence and due to application of certain herbicides (like atrazine or paraquat).

“Increased stress tolerance” as used herein comprises, for any given stress, but particularly for oxidative stress, increasing tolerance in a(n) (eukaryotic) cell, tissue, organ or organism to oxidative stress conditions, whether said (eukaryotic) cell, tissue, organ or organism already have some degree of tolerance to the particular stress, such as oxidative stress, or whether said (eukaryotic) cell, tissue, organ or organism is being provided with tolerance to that stress, particularly oxidative stress, anew. Particularly, said increased stress tolerance is in the meaning of increased oxidative stress tolerance, and/or increased viability and cell survival under oxidative stress conditions, but it is understood that due to cross-tolerance said tolerance to oxidative of a cell or organism may go hand in hand with an increased tolerance of a cell or organism to other stress conditions as well, particularly abiotic or environmental stress conditions including metal toxicity, temperature stress (i.e. stress induced by sub-optimal or supra-optimal growth temperatures for a particular cell or organism), osmotic stress (i.e. any stress associated with or induced by loss of water, reduced turgor or reduced water content of a cell, tissue, organ or organism), drought stress (i.e. any stress which is induced by or associated with the deprivation of water or reduced supply of water to a cell, tissue, organ or organism) or salt stress (i.e. any stress which is associated with or induced by elevated concentrations of salt or ions in general and which result in a perturbation in the osmotic potential of the intracellular or extracellular environment of a cell).

The terms “tolerance” and “resistance” as used herein encompass protection against stress ranging from a delay to substantially a complete inhibition of alteration in cellular metabolism, reduced cell growth and/or cell death caused by stress conditions, particularly oxidative stress conditions.

By “isolated” it is meant material that is substantially or essentially free from components that normally accompany it in its native state used or that is essentially free of other cellular materials or culture medium when produced by recombinant techniques, or substantially free of chemical precursors when chemically synthesized. For example, an “isolated poly- or oligonucleotide”, as used herein, refers to a poly- or oligonucleotide, which has been purified from the sequences which flank it in a naturally-occurring state.

The terms “oligonucleotide”, “polynucleotide” or “nucleic acid” as used herein refers to a polymer composed of a multiplicity of nucleotide units (deoxyribonucleotides or ribonucleotides, or related structural variants or synthetic analogues thereof) linked via phosphodiester bonds (or related structural variants or synthetic analogues thereof). An oligonucleotide is typically rather short in length, generally from about 10 to 30 nucleotides, but these terms can refer to nucleic acid molecules of any length, although the term “polynucleotide” is typically used for large oligonucleotides and typically refers to nucleic acid polymers greater than 30 nucleotides in length. The term “polynucleotide” or “nucleic acid” as used herein designates mRNA, RNA, cRNA, cDNA or DNA.

The term “recombinant nucleic acid” as used herein refers to a nucleic acid polymer formed in vitro by the manipulation of nucleic acid into a form not normally found in nature. For example, the recombinant nucleic acid may be in the form of an expression vector. Generally, such expression vectors include transcriptional and translational regulatory nucleic acid operably linked to the nucleotide sequence.

The terms “peptide” or “polypeptide” refer to a polymer of amino acid residues and to variants and synthetic analogues of the same, encompassing native peptides (including synthetically synthesized or recombinant peptides) and peptidomimetics (typically, synthetically synthesized peptides). Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers. A peptide of the present invention may also be produced by recombinant expression in prokaryotic and eukaryotic engineered cells other than plant cells, such as bacteria, fungi, or animal cells. Suitable expression systems are known to those skilled in the art. By “recombinant (poly)peptide” is meant a (poly)peptide made using recombinant techniques, i.e., through the expression of a recombinant or synthetic polynucleotide. When the polypeptide is recombinantly produced, it is also preferably substantially free of culture medium, i.e., culture medium represents less than about 20%, more preferably less than about 10%, and most preferably less than about 5% of the volume of the peptide preparation. The term “peptide” also refers to modified peptides wherein the modifications render the peptides even more stable e.g. while in a body. Such modifications include, but are not limited to N-terminus modification, C-terminus modification, peptide bond modification, including, but not limited to, CH₂—NH, CH₂—S, CH₂—S=0, 0=C—NH, CH₂-0, CH₂—CH₂, S═C—NH, CH═CH or CF═CH, backbone modifications, and residue modification.

Methods for preparing peptidomimetic compounds are well known in the art and are specified, for example, in Quantitative Drug Design, C. A. Ramsden Gd., Chapter 17.2, F. Choplin Pergamon Press (1992) (incorporated herein by reference).

“Homologues” of a peptide include peptides, oligopeptides, polypeptides or proteins having amino acid substitutions, deletions and/or insertions relative to the unmodified peptide in question and having similar biological and functional activity as the unmodified protein from which they are derived. To produce such homologues, amino acids of the protein may be replaced by other amino acids having similar properties (“conservative substitution”) (such as similar hydrophobicity, hydrophilicity, antigenicity, propensity to form or break α-helical structures or β-sheet structures). Conservative substitution tables are well known in the art (see for example Creighton (1984) Proteins. W.H. Freeman and Company, incorporated herein by reference). The homologues useful in the present invention have at least 70% sequence identity or similarity (functional identity) to the unmodified peptide, preferably at least 80% or 90%, more preferably at least 95%, 97%, 99% sequence identity or similarity to an unmodified peptide of the invention. Furthermore, homologues of a peptide according to the present invention are capable of increasing the oxidative stress tolerance or viability of a cell or organism under oxidative stress conditions.

The term “sequence identity” as used herein refers to the extent that sequences are identical on a nucleotide-by-nucleotide basis or an amino acid-by-amino acid basis over a window of comparison. Sequence identity is generally determined by aligning the residues of the two sequences to optimize the number of identical amino acids or nucleotides along the lengths of their sequences; gaps in either or both sequences are permitted in making the alignment in order to optimize the number of identical residues, although the amino acids or nucleotides in each sequence must nonetheless remain in their proper order. Thus, a “percentage of sequence identity” is calculated by comparing two optimally aligned sequences over the window of comparison, determining the number of positions at which the identical nucleic acid base (e.g., A, T, C, G, I, U) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Preferably, sequence identity between two amino acid or two nucleotide sequences is determined by comparing said sequences using the Blastp or Blastn program, respectively, available at http://blast.ncbi.nlm.nih.gov/Blast.cgi. Preferably, the default values for all BLAST 2 search parameters are used, including in the case of Blastp: matrix=BLOSUM62; open gap penalty=11, extension gap penalty=1, gap x-dropoff=50, expect=10, wordsize=3, and filter on; and in the case of Blastn: [i.e. “Expect threshold”=10; “word size”=11; “Match/mismatch scores”=(2,−3); “Gap costs”=(existence: 5−extension: 2); filter for low complexity regions & mask for lookup table only). “Similarity” refers to the percentage number of amino acids that are identical or constitute conservative substitutions.

The term “expression cassette” refers to any recombinant expression system for the purpose of expressing a nucleic acid sequence of the invention in vitro or in vivo, constitutively or inducibly, in any cell, including, in addition to plant cells, prokaryotic, yeast, fungal, insect or mammalian cells. The term includes linear and circular expression systems. The term includes all vectors. The cassettes can remain episomal or integrate into the host cell genome. The expression cassettes can have the ability to self-replicate or not (i.e. drive only transient expression in a cell). The term includes recombinant expression cassettes that contain only the minimum elements needed for transcription of the recombinant nucleic acid.

A “nucleotide sequence encoding a peptide” (i.e. a gene, coding sequence, open reading frame or ORF) is a nucleotide sequence that can be transcribed into mRNA and/or translated into a polypeptide when present in an expressible format, i.e. when the coding sequence or ORF is placed under the control of appropriate control sequences or regulatory sequences. A coding sequence or ORF is bounded by a 5′ translation start codon and a 3′ translation stop codon. A coding sequence or ORF can include, but is not limited to RNA, mRNA, cDNA, recombinant nucleotide sequences, synthetically manufactured nucleotide sequences or genomic DNA. The coding sequence or ORF can be interrupted by intervening nucleic acids.

### Peptide for Increasing the Oxidative Stress Tolerance of a Host Cell or Organism

A first object of the present invention relates to an isolated peptide (also herein referred to as “peptide of the present invention”) comprising an amino acid sequence which is at least 70%, more preferably at least 80%, more preferably at least 90%, more preferably at least 95%, 97%, 99% or 100% identical to an amino acid sequence selected from the amino acid sequences listed in Table 1, i.e an amino acid sequence selected from SEQ ID No. 1 to SEQ ID No. 575, wherein said isolated peptide increases the tolerance of a host cell or organism to oxidative stress conditions, or wherein said isolated peptide increases the cell viability under oxidative stress conditions or in the presence of agents inducing mitochondrial dysfunction.

In a preferred embodiment of the present invention, said isolated peptide that increases the tolerance of a host cell or organism to oxidative stress conditions comprise an amino acid sequence which is at least 70% or 80%, more preferably at least 90%, more preferably at least 95%, 97%, 99% or 100% identical to amino acid sequence SEQ ID No. 1; SEQ ID No. 2; SEQ ID No. 3; SEQ ID No. 4; SEQ ID No. 5; SEQ ID No. 6 or SEQ ID No. 7 (Table 3). Most preferably, said isolated peptide that increases the tolerance of a host cell or organism to oxidative stress conditions comprise an amino acid sequence which is at least 70% or 80%, more preferably at least 90%, more preferably at least 95%, 97%, 99% or 100% identical to amino acid sequence SEQ ID No. 1.

TABLE 3
List of validated peptides that increase oxidative stress tolerance

| CODE | AMINO ACID CODE | SEQ ID NO |
|---|---|---|
| (O)SIP108 | MLCVLQGLRE | SEQ ID No. 1 |
| (O)SIP14_1 | MIIINNDNYLLLFYNNN | SEQ ID No. 2 |
| (O)SIP14_2 | MIIIYYCFIIIIN | SEQ ID No. 3 |
| (O)SIP11 | MLMYRMRSGAN | SEQ ID No. 4 |
| (O)SIP163 | MGLNEDSVFRSIKPFKSP | SEQ ID No. 5 |
| (O)SIP152 | MNVLARAPRLRHQLQNLTQDRRKTQMQMKGQRVRTTSLQ | SEQ ID No. 6 |
| (O)SIP37 | MAREEKEQSVYDIYTFASLLL | SEQ ID No. 7 |

A particular embodiment relates to a derivative of a peptide of the present invention, such as but not limited to, an allelic variant, a homologue or a mutation of a peptide of the present invention. Preferably, a derivative, variant, mutation or homologue of a peptide of the present invention has at least the same or better functional activity than the unmodified peptide, such as the capability of increasing the oxidative stress tolerance of a host cell or organism, and/or the capacity to increase the cell viability and cell survival under oxidative stress conditions and/or in the presence of an agent inducing mitochondrial dysfunction. The functional activity can be tested with for example the methods as described in Example 2, 3 and 4, e.g. evaluating the peroxide MIC values or halo formation following overexpression of the modified peptide in yeast, or measuring the ROS accumulation following exogeneous application of the modified peptide to a yeast culture.

The term “derivative(s) of a peptide” refers to peptides or polypeptides which, compared to the amino acid of a naturally-occurring form of the peptide as presented in SEQ ID No. 1 to SEQ ID No. 575, preferably as presented in SEQ ID No. 1, SEQ ID No. 2, SEQ ID No. 3, SEQ ID No. 4, SEQ ID No. 5, SEQ ID No. 6 or SEQ ID No. 7, most preferably as presented in SEQ ID No. 1 may comprise: (i) substitutions, deletions or additions of naturally and non-naturally occurring amino acid residues; (ii) amino acid residues that are substituted by corresponding naturally or non-naturally altered amino acids; (iii) naturally occurring altered, (such as glycosylated, acylated, myristoylated or phosphorylated amino acids) or non-naturally occurring amino acid residues (such as biotinylated amino acids, or amino acids modified after CNBr treatment); (iv) peptides carrying post-translational modifications. A derivative may also comprise one or more non-amino acid substituents compared to the amino acid from which it is derived, for example a reporter molecule or other ligand, covalently or non-covalently bound to the amino acid such as, for example, a reporter molecule which is bound to facilitate its detection. Preferably, amino acid substitutions comprise conservative amino acid substitutions. One or more amino acid residues may be introduced into a predetermined site in said peptide of the present invention. Insertions can comprise amino-terminal and/or carboxy-terminal fusions as well as intra-sequence insertions of single or multiple amino acids. Examples of amino- or carboxy-terminal fusion proteins or peptides include the binding domain or activation domain of a transcriptional activator as used in the yeast two-hybrid system, phage coat proteins, (histidine)₆-tag, glutathione S-transferase-tag, protein A, maltose-binding protein, dihydrofolate reductase, Tag•100 epitope, c-myc epitope, FLAG®-epitope, lacZ, CMP (calmodulin-binding peptide), HA epitope, protein C epitope and VSV epitope.

In a particular embodiment the invention relates to a method for recombinant production of a peptide of the invention, comprising introducing an expression cassette comprising a nucleic acid encoding an oxidative stress tolerance inducing peptide of the invention, introducing said expression cassette in a suitable host cell, culturing the resulting recombinant host under suitable conditions and isolating the oxidative stress tolerance inducing peptide produced. A preferred method involves the synthesis of nucleic acid sequences by PCR and its insertion into said expression vector and subsequently transfecting or transforming a suitable host cell with the expression vector. A large number of suitable methods exist in the art to produce peptides in appropriate hosts. If the host is a unicellular organism such as a prokaryote or a mammalian or insect cell, the person skilled in the art can revert to a variety of culture conditions. To increase the yield and the solubility of the expression product, the medium can be buffered or supplemented with suitable additives known to enhance or facilitate both. In general, the skilled person is also aware that the culture and temperature conditions may have to be adapted to the needs of the host and the requirements of the peptide expressed. In case an inducible promoter controls the nucleic acid of the invention in the vector present in the host cell, expression of the polypeptide can be induced by addition of an appropriate inducing agent. Conveniently, the produced protein is harvested from the culture medium, lysates of the cultured cells or from isolated (biological) membranes by established techniques. Suitable expression protocols and strategies are known to the skilled person and can be retrieved e.g. from Sambrook, 2001. In a particular embodiment when recombinantly producing the peptides of the invention in a host cell, the expression vector may encode a fusion peptide or fusion polypeptide e.g. wherein the peptide of the invention is coupled to a signal peptide to direct expression to a specific compartment or site or to a tag which facilitates purification of the fusion peptide or polypeptide. Suitable tags are well known in the art and comprise e.g. a hexahistidine tag and a GST (glutathione S-transferase) tag. The fusion peptide or fusion polypeptide expressed may be further processed in order to cleave the compensating peptide or polypeptide or the signal peptide or tag fused to the peptide of the invention. This can take place at any stage of the purification process after culturing the host cell. Suitable methods to cleave off the undesired part are either chemical methods using e.g. cyanogen bromide or N-chloro succinimide, or, preferably, enzymatic methods, using e.g. proteases suitable for cleavage specific for a certain amino acid sequence and include Factor Xa or TEV protease.

In another particular embodiment a peptide of the present invention can be produced synthetically. Chemical synthesis of peptides is well known in the art. Solid phase synthesis is commonly used and various commercial synthesizers are available, for example automated synthesizers by Applied Biosystems Inc.; Beckman; MultiSyntech; GenScript, Jena etc. Solution phase synthetic methods, as known by the person skilled in the art, may also be used, although they are less convenient. The peptides are commonly analysed by matrix-associated laser desorption time-of-flight mass spectrometry. By using these standard techniques, naturally occurring amino acids may be substituted with unnatural amino acids, particularly D-stereoisomers, and also with amino acids with side chains having different lengths or functionalities. Functional groups for conjugating to small molecules, label moieties, peptides, or proteins may be introduced into the molecule during chemical synthesis. In addition, small molecules and label moieties may be attached during the synthesis process. Preferably, introduction of the functional groups and conjugation to other molecules minimally affect the structure and function of the subject peptide.

In specific embodiments the N- and C-terminus of an oxidative stress tolerance inducing peptide of the present invention may be derivatized using conventional chemical synthesis methods. The peptides of the invention may contain an acyl group, such as an acetyl group. Methods for acylating, and specifically for acetylating the free amino group at the N-terminus are well known in the art. For the C-terminus, the carboxyl group may be modified by esterification with alcohols or amidated to form —CONH₂ or CONHR. Methods of esterification and amidation are well known in the art.

Furthermore, an oxidative stress tolerance inducing peptide of the present invention may also be produced semi-synthetically, for example by a combination of recombinant and synthetic production. In the case that fragments of the peptides are produced synthetically, the remaining part of the peptide would have to be produced otherwise, e.g. recombinantly as described further below, and then be linked to the fragment to form the peptide of the invention.

Furthermore, the invention encompasses peptidomimetics of the peptide as defined above. A peptidomimetic is a small protein- or peptide-like chain designed to mimic a peptide. Peptidomimetics typically arise from modifications of an existing peptide in order to alter the properties of the peptide. For example, they may arise from modifications to change the stability of the peptide. These modifications involve changes to the peptide that will not occur naturally (such as altered backbones and the incorporation of non-natural amino acids), including the replacement of amino acids or peptide bonds by functional analogues. Such functional analogues include all known amino acids other than the 20 gene-encoded amino acids, such as for example selenocysteine. The use of peptidomimetics as compared to other mimetics has some particular advantages, including (i) their conformationally restrained structure allows to minimize binding to non-target compounds and to enhance the activity at the desired targets; (ii) through the addition of hydrophobic residues and/or replacement of amide bonds the transport of peptidomimetics through cellular membranes can be improved; (iii) peptidomimetics (e.g. cyclic peptides) are less susceptible to degradation by peptidases and other enzymes.

Yet another alternative method for producing the peptide of the invention is in vitro translation of mRNA. Suitable cell-free expression systems for use in accordance with the present invention include rabbit reticulocyte lysate, wheat germ extract, canine pancreatic microsomal membranes, E. coli S30 extract, and coupled transcription/translation systems such as the TNT-system (Promega). These systems allow the expression of recombinant peptides upon the addition of cloning vectors, DNA fragments, or RNA sequences containing coding regions and appropriate promoter elements.

Methods of isolation of the peptide produced are well-known in the art and comprise, without limitation, method steps such as ion exchange chromatography, gel filtration chromatography (size exclusion chromatography), affinity chromatography, high pressure liquid chromatography (HPLC), reversed phase HPLC, disc gel electrophoresis or immunoprecipitation (see, for example, Sambrook, 2001).

Nucleic Acid Encoding for a Peptide that Increases the Oxidative Stress Tolerance of a Host Cell or Organism

Another embodiment of the present invention provides a nucleic acid that can be used to confer oxidative stress tolerance or resistance to a host cell or organism, and/or to confer increased cell viability under oxidative stress conditions or in the present of agents that induce mitochondrial dysfunction. The present invention therefore also provides an isolated nucleic acid encoding a peptide of the present invention as defined above, the complement thereof or a part thereof.

In a particular embodiment the invention provides a nucleotide sequence (also referred to as “nucleic acid of the (present) invention”) capable of encoding an oxidative stress tolerance increasing peptide comprising an amino acid sequence selected from SEQ ID No. 1 to SEQ ID No. 575, or a nucleotide sequence encoding a homologue having at least 70% or 80%, more preferably at least 90%, more preferably at least 95%, 97%, 99% or 100% identity with an amino acid sequence selected from SEQ ID No. 1 to SEQ ID No. 575. Preferably, said nucleotide sequence according to the present invention is capable of encoding an oxidative stress tolerance increasing peptide comprising an amino acid sequence selected from SEQ ID No. 1 (e.g. nucleotide sequence SEQ ID No. 576); SEQ ID No. 2 (e.g. nucleotide sequence SEQ ID No. 577); SEQ ID No. 3 (e.g. nucleotide sequence SEQ ID No. 578); SEQ ID No. 4 (e.g. nucleotide sequence SEQ ID No. 579); SEQ ID No. 5 (e.g. nucleotide sequence SEQ ID No. 580); SEQ ID No. 6 (e.g. nucleotide sequence SEQ ID No. 581) and/or SEQ ID No. 7 (e.g. nucleotide sequence SEQ ID No. 582), or a nucleotide sequence encoding a homologue having at least 70% or 80%, more preferably at least 90%, more preferably at least 95%, 97%, 99% or 100% identity with an amino acid sequence selected from SEQ ID No. 1; SEQ ID No. 2; SEQ ID No. 3; SEQ ID No. 4; SEQ ID No. 5; SEQ ID No. 6 and SEQ ID No. 7. Most preferably, said nucleotide sequence according to the present invention is capable of encoding an oxidative stress tolerance increasing peptide comprising amino acid sequence SEQ ID No. 1, or a nucleotide sequence encoding a homologue having at least 70% or 80%, more preferably at least 90%, more preferably at least 95%, 97%, 99% or 100% identity with amino acid sequence SEQ ID No. 1.

Preferably, said nucleotide sequence according to the present invention has at least 70% or 80%, more preferably at least 90%, more preferably at least 95%, 97%, 99% or 100% identity with a nucleotide sequence selected from SEQ ID No. 576, SEQ ID No. 577, SEQ ID No. 578, SEQ ID No. 579, SEQ ID No. 580, SEQ ID No. 581 and SEQ ID No. 582 (Table 4), or a complement thereof. More preferably, said nucleotide sequence of the present invention has at least 70% or 80%, more preferably at least 90%, more preferably at least 95%, 97%, 99% or 100% identity with a nucleotide sequence selected from SEQ ID No. 576, or a complement thereof.

Advantageously, the nucleic acids according to the invention may be produced using recombinant or synthetic means, such as, for example, PCR cloning mechanisms. Generally, such techniques as defined herein are well known in the art, for example as described in Sambrook et al. (Molecular Cloning: a Laboratory Manual, 2001). Polynucleotides may also be synthesized by well-known techniques as described in the technical literature [see e.g. 15, 16]. Double stranded DNA fragments may then be obtained either by synthesizing the complementary strand and annealing the strands together under appropriate conditions, or by adding the complementary strand using DNA polymerase with an appropriate primer sequence.

TABLE 4
List of nucleic acids encoding for validated oxidative stress tolerance
increasing peptides

| CODE | AMINO ACID CODE | SEQ ID NO |
|---|---|---|
| (O)SIP108 | atgctatgtgtgcttcaaggtttaagggag | SEQ ID No. 576 |
| (O)SIP14_1 | atgataattattaataatgataattatttattattgttttataataataat | SEQ ID No. 577 |
| (O)SIP14_2 | atgataattatttattattgttttataataataattaat | SEQ ID No. 578 |
| (O)SIP11 | atgctcatgtaccggatgagaagcggagcaaac | SEQ ID No. 579 |
| (O)SIP163 | atgggcttaaacgaggatagtgtgtttcgtagcataaagccttttaaaagccca | SEQ ID No. 580 |
| (O)SIP152 | atgaatgtattagcaagagctccacggctccgtcatcagctccagaatctgaca | SEQ ID No. 581 |
| | caagatcggagaaaaactcaaatgcagatgaagggtcaacgagtcagaacga | |
| | cgtcgctgcag | |
| (O)SIP37 | atggcgagagaagaaaaagaacaatctgtgtatgatatttacacgtttgcgtct | SEQ ID No. 582 |
| | ctgctattg | |

### Use of the Oxidative Stress Tolerance Inducing Agent of the Invention

The present invention further relates to the use of a peptide or peptidomimetic of the invention or a nucleic acid encoding for said peptide of the present invention, or a homologue or derivative for increasing the oxidative stress tolerance of a host cell and/or for increasing the viability or survivability of a cell under oxidative stress conditions or in the presence of an agent inducing mitochondrial dysfunction. In an embodiment the present invention relates to the use of a peptide or peptidomimetic of the invention or a nucleic acid encoding for said peptide of the present invention, or a homologue or derivative thereof for the prevention or treatment of oxidative stress related disorders (including but not limited to mitochondrial dysfunction disorders) or oxidative stress related cellular (mitochondrial) damage in an organism, such as a microbial organism or a plant or mammal, such as a human or to improve the oxidative stress tolerance or resistance of a host cell or an organism, such as a microbial organism or a plant or mammal. Alternatively, the present invention relates to a peptide or peptidomimetic of the present invention or derivative thereof or a nucleic acid encoding therefor for use as an oxidative stress tolerance inducing agent. An “oxidative stress tolerance inducing agent” is in the meaning that said peptide, peptidomimetic or nucleic acid of the present invention (or a derivative thereof) is able to (i) increase, enhance or improve the oxidative stress tolerance of a host cell or an organism, preferably an eukaryotic cell or organism (mammal, plant or microbial organism); and/or (ii) increase the viability of a host cell in oxidative stress conditions or in the presence of an agent inducing mitochondrial dysfunction; and/or (iii) protect a host cell or organism against oxidative stress; (iv) and/or be used for treating, ameliorating and/or preventing of oxidative stress related disorders, particularly mitochondrial dysfunction disorders, in an organism, such as a mammal, human or plant. It is understood that the terms “tolerance” and “resistance” are used interchangeably herein. Said oxidative stress tolerance inducing agent may effectively decrease or prevent an increase in the ROS levels in the cell. However, not all individual ROS species monitored would necessarily have to demonstrate a decrease or lack of an increase in its level. Particularly, said oxidative stress tolerance inducing agent may effectively increase or prevent a decrease in viability or survivability of a cell in oxidative stress conditions and/or in the presence of an agent inducing mitochondrial dysfunction. It is understood that due to cross-tolerance said oxidative stress tolerance inducing agent may increase the tolerance of a cell or organism to other abiotic or environmental stress conditions as well.

A particular embodiment of the present invention relates to a method for increasing or inducing the oxidative stress tolerance of a cell or non-human organism, preferably an eukaryotic cell, such as a fungal cell, yeast or a plant cell, comprising introducing into said cell a chimeric genetic construct comprising a nucleic acid capable of encoding an oxidative stress tolerance increasing peptide of the present invention. Preferably, said nucleic acid capable of encoding an oxidative stress tolerance increasing peptide is operably linked to a promoter that drives expression of a coding sequence in said cell. Preferably, said chimeric genetic construct is stably incorporated in the genome of said cell.

Another particular embodiment of the present invention relates to a method for inducing or increasing the oxidative stress tolerance or resistance of a cell or increasing the viability of a cell under oxidative stress conditions, preferably an eukaryotic cell, by contacting said cell with an oxidative stress tolerance inducing agent according to the present invention, preferably contacting said cell with a peptide or peptidomimetic of the invention or a derivative thereof. The cell can be a microbial cell, such as yeast or a fungal cell, a plant cell, a mammal or human cell, in a cell culture, a tissue, an organ, or an organism. Hence, this method can be carried out in vivo or in vitro.

Yet another embodiment of the present invention relates to a peptide of the present invention or a nucleic acid encoding therefor for use in preventing or treating an oxidative stress-related disorder, preferably a mitochondrial dysfunction disorder. Thus, the present invention provides a method of treating an oxidative stress-related disorder in a subject in need thereof, the method comprising administering to the subject a therapeutically effective amount of the pharmaceutical composition of the present invention, thereby treating the oxidative stress-related disorder.

In another particular embodiment of the present invention a method for affecting or increasing the lifespan of one or more (eukaryotic) cells is provided, comprising the use of a peptide of the present invention, a peptidomimetic thereof or a nucleic acid encoding therefor. The eukaryotic cell can be a cultured cell, such as a tissue cell culture, or the cell can be one of many cells in, for example, a tissue, or an organ, or a biological system, such as a mammal or a human. Thus, the lifespan of a tissue or an organ, for example, a tissue or an organ that is intended for transplantation, can be extended (relative to a similar cell, tissue, organ or biological system that does not receive a peptide or composition of the invention) using the method of the invention. Lifespan can include the number of times a cell or cell population can divide (replicative lifespan), or the length of time a cell or organism survives before dying (chronological lifespan).

### Increasing Oxidative Stress Tolerance of Eukaryotic Cells & Organisms

A particular embodiment of the present invention encompasses a genetic modification of a host cell or non-human organism, preferably an eukaryotic microbial cell or organism, a plant or a plant cell. The term “genetic modification” refers to a change by human intervention in the genetic content of a cell compared to a wild type cell and includes techniques like genetic engineering, breeding or mutagenesis. The change in genetic content comprises modifications of the genome and includes addition, deletion and substitution of genetic material in the chromosomes of said cell as well as in episomes. The term also encompasses the addition of extrachromosomal information to said cell. Preferably, the genetic modification results in modulated, preferably increased, expression of a nucleic acid. Genetic modification typically includes a selection step, during which the cells or organisms, preferably eukaryotic microbial cells or organisms, or plants or plant cells, with the desired characteristics are selected.

Thus, a particular embodiment of the present invention relates to a method for increasing the oxidative stress tolerance of a cell, preferably an eukaryotic cell, including but not limited to a fungal cell, yeast or a plant cell, comprising introducing into said cell a chimeric genetic construct comprising a nucleic acid capable of encoding an oxidative stress tolerance increasing peptide of the present invention.

The present invention thus also relates to a recombinant genetic construct or chimeric gene comprising a nucleic acid according to the invention operably linked to one or more regulatory elements. The genetic constructs facilitate the introduction and/or expression and/or maintenance of a nucleotide sequence as defined above into said cell or non-human organism, such as an eukaryotic microbial cell, a plant cell, tissue or organ. Preferably, the chimeric genetic construct comprises (i) an isolated nucleic acid encoding a peptide comprising a sequence having at least 70%, preferably 80% or 90%, more preferably 95%, 96%, 97%, 98%, 99% or 100% sequence identity or similarity to the amino acid sequence as given in SEQ ID No. 1, SEQ ID No. 2, SEQ ID No. 3, SEQ ID No. 4, SEQ ID No. 5, SEQ ID No. 6 or SEQ ID No. 7, preferably as given in SEQ ID No. 1, or a complement thereof; (ii) a regulatory element operably linked to the nucleic acid of (i), and optionally (iii) a 3′ end region comprising a transcription termination sequence. Preferably, said regulatory element of (ii) is a regulatory element functional in said cell, e.g. a plant- or yeast-expressible promoter in the case of a plant or yeast cell, respectively. Preferably, said transcription termination sequence of (iii) is functional in said eukaryotic cell.

The term “operably linked” as used herein refers to a functional linkage between the regulatory element (or the termination sequence) and the gene or nucleic acid of interest, such that the regulatory element is able to initiate transcription of the gene or nucleic acid of interest.

For expression in a host cell, the nucleic acid molecule must be linked operably to or comprise a suitable promoter which expresses the gene at the right point in time and with the required spatial expression pattern. As used herein, the term “plant-expressible promoter” refers to a promoter that is capable of driving transcription in a plant cell. This not only includes any promoter of plant origin, but also any promoter of non-plant origin which is capable of directing transcription in a plant cell. The promoter may also be an artificial or synthetic promoter. The term “promoter”, such as a “plant-expressible promoter”, includes, but is not restricted to, constitutive, inducible, organ-, tissue- or cell-specific and/or developmentally regulated promoters. The terms “regulatory element”, “control sequence”, “promoter” are all used herein interchangeably and, taken in a broad context, refer to regulatory nucleic acids capable of effecting expression of the sequences to which they are ligated. Similarly, the term “yeast-expressible promoter” refers to a promoter that is capable of driving transcription in a yeast cell and encompasses natural yeast promoters as well as other promoter sequences capable of driving expression in yeast cells. Suitable promoters for expression in yeast are known in the art, see for example Current Protocols in Molecular Biology, Unit 13 (Ausubel et al., 1994) and the Guide to Yeast Genetics and Molecular Biology (Guthrie and Fink, 1991) (incorporated herein by reference).

A “constitutive promoter” refers to a promoter that is transcriptionally active during most, but not necessarily all, phases of growth and development and under most environmental conditions, in at least one cell, tissue or organ, preferably in the majority of tissues or cells of an organism. A developmentally-regulated promoter is active during certain developmental stages or in parts of the plant or organism that undergo developmental changes. An inducible promoter has induced or increased transcription initiation in response to a chemical (for a review see Gatz 1997, Annu. Rev. Plant Physiol. Plant Mol. Biol., 48: 89-108), environmental or physical stimulus, or may be “stress-inducible”, i.e. activated when a plant or cell is exposed to various stress conditions, or a “pathogen-inducible” i.e. activated when a plant is exposed to exposure to various pathogens. An organ-specific or tissue-specific promoter is one that is capable of preferentially initiating transcription in certain organs or tissues, such as the leaves, roots, seed tissue etc in case of a plant. In any case, the particular promoter selected to drive the expression of the oxidative stress tolerance inducing peptides of the invention in transgenic plants should be capable of causing sufficient expression of these peptides to result in the production of an effective amount of the peptides in plant tissues to confer an increased resistance to stress conditions in said plant tissues. Examples of constitutive promoters capable of driving such expression are the 35S, rice actin, maize ubiquitin, and elF-4A promoters or the promoter of the nopaline synthase gene (“PNOS”) of the Ti-plasmid or the promoter of the octopine synthase gene.

Optionally, one or more terminator sequences may also be used in the construct introduced into said cell, preferably an eukaryotic cell, such as a plant, fungal, yeast or mammal cell. The term “terminator” encompasses a control sequence which is a DNA sequence, at the end of a transcriptional unit, which signals 3′ processing and polyadenylation of a primary transcript and termination of transcription. The terminator can be derived from the natural gene, from a variety of other (plant) genes, or from T-DNA. The terminator to be added may be derived from, for example, the nopaline synthase or octopine synthase genes, or alternatively from another eukaryotic (plant) gene.

Additional regulatory elements may include transcriptional as well as translational enhancers. Those skilled in the art will be aware of terminator and enhancer sequences which may be suitable for use in performing the invention. Methods for increasing expression of genes or gene products are well documented in the art and include, for example, overexpression driven by appropriate promoters (as described herein), the use of transcription enhancers or translation enhancers. Isolated nucleic acids which serve as promoter or enhancer elements may be introduced in an appropriate position (typically upstream) of a non-heterologous form of a polynucleotide so as to upregulate expression of a nucleic acid encoding the peptide of interest. An intron sequence may also be added to the 5′ untranslated region (UTR) or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold [17, 18]. Such intron enhancement of gene expression is typically greatest when placed near the 5′ end of the transcription unit. Use of the maize introns Adh1-S intron 1, 2, and 6, the Bronze-1 intron are known in the art. For general information see: The Maize Handbook, Chapter 116, Freeling and Walbot, Eds., Springer, N.Y. (1994).

Furthermore, the recombinant nucleic acid can be constructed and employed to target the gene product of the nucleic acid of the invention to a specific intracellular compartment within said cell or to direct said peptide to the extracellular environment. This can generally be obtained by operably joining a DNA sequence encoding a transit or signal peptide to the recombinant nucleic acid.

The genetic construct may optionally comprise a selectable marker gene. As used herein, the term “selectable marker gene” includes any gene which confers a phenotype on a cell in which it is expressed to facilitate the identification and/or selection of cells which are transfected or transformed with a genetic construct of the invention or a derivative thereof. Suitable markers may be selected from markers that confer antibiotic resistance. Cells containing the recombinant DNA will thus be able to survive in the presence of antibiotic concentrations that kill untransformed cells. Examples of selectable marker genes include genes conferring resistance to antibiotics (such as nptII that phosphorylates neomycin and kanamycin, or hpt, phosphorylating hygromycin, or genes conferring resistance to, for example, bleomycin, streptomycin, tetracyclin, chloramphenicol, ampicillin, gentamycin, geneticin (G418), spectinomycin or blasticidin), to herbicides (for example bar which provides resistance to Basta®; aroA or gox providing resistance against glyphosate, or the genes conferring resistance to, for example, imidazolinone, phosphinothricin or sulfonylurea), or genes that provide a metabolic trait (such as manA that allows plants to use mannose as sole carbon source or xylose isomerase for the utilisation of xylose, or antinutritive markers such as the resistance to 2-deoxyglucose). Expression of visual marker genes results in the formation of colour (for example β-glucuronidase, GUS or β-galactosidase with its coloured substrates, for example X-Gal), luminescence (such as the luciferin/luceferase system) or fluorescence (Green Fluorescent Protein, GFP, and derivatives thereof). This list represents only a small number of possible markers. The skilled worker is familiar with such markers and different markers will be preferred, depending on the organism and the selection method.

Since the marker genes, particularly genes for resistance to antibiotics, are no longer required or are generally undesired in the transgenic host cell once the nucleic acids have been introduced successfully, the process according to the invention for introducing the nucleic acids advantageously employs techniques which enable the removal or excision of these marker genes. One such a method is what is known as co-transformation. The co-transformation method employs two vectors simultaneously for the transformation, one vector bearing the nucleic acid according to the invention and a second bearing the marker gene(s). A large proportion of transformants receives or comprises both vectors. In case of (plant) transformation with Agrobacteria, the transformants usually receive only a part of the vector, i.e. the sequence flanked by the T-DNA, which usually represents the expression cassette. The marker genes can subsequently be removed from the transformed organism (plant) by performing crosses. In another method, marker genes integrated into a transposon are used for the transformation together with desired nucleic acid (known as the Ac/Ds technology). The transformants can be crossed with a transposase source or the transformants are transformed with a nucleic acid construct conferring expression of a transposase, transiently or stable. In some cases (approx. 10%), the transposon jumps out of the genome of the host cell once transformation has taken place successfully and is lost. A further advantageous method relies on what is known as recombination systems, e.g. the Cre/lox system whereby elimination of the marker gene by crossing is no longer necessary. Cre1 is a recombinase that removes the sequences located between the loxP sequences. If the marker gene is integrated between the loxP sequences, it is removed once transformation has taken place successfully, by expression of the recombinase. Further recombination systems are the HIN/HIX, FLP/FRT and REP/STB system (Tribble et al., J. Biol. Chem., 275, 2000: 22255-22267; Velmurugan et al., J. Cell Biol., 149, 2000: 553-566). A site-specific integration into the (plant) genome of the nucleic acid sequences according to the invention is also contemplated.

According to another embodiment, the present invention relates to the use of the nucleic acid encoding a peptide of the present invention or the use of such a peptide as selectable marker gene in plants or other organisms, with selection taking place by treating with a suitable oxidative stress condition, such as in the presence of paraquat.

Thus, a particular embodiment of the present invention relates to a method for increasing the oxidative stress tolerance of an cell or organism, preferably an eukaryotic cell or non-human organism comprising introducing into said cell or (non-human) organism a chimeric genetic construct comprising a nucleic acid capable of encoding an oxidative stress tolerance increasing peptide comprising an amino acid sequence having at least 70%, preferably 80% or 90%, more preferably 95%, 96%, 97%, 98%, 99% or 100% sequence identity or similarity to the amino acid sequence as given in SEQ ID No. 1, SEQ ID No. 2, SEQ ID No. 3, SEQ ID No. 4, SEQ ID No. 5, SEQ ID No. 6 or SEQ ID No. 7, preferably as given in SEQ ID No. 1. Preferably, said eukaryotic cell or organism is a fungal cell or yeast, or a plant cell or plant, or a mammal cell. Advantageously, a transgenic microbial eukaryotic cell or organism, plant cell or plant obtained by the methods of the present invention is tolerant or resistant to oxidative stress conditions and/or has increased survivability in oxidative stress conditions or in the presence of agents that induce mitochondrial dysfunction.

The term “transformation” or “introduction” as referred to herein means the transfer of an exogenous or foreign polynucleotide or gene into a cell, irrespective of the method used for transfer. The polynucleotide may be transiently or stably introduced into said cell and may be maintained non-integrated, for example, as a plasmid, or alternatively, may be integrated into the host genome. Transformation of a cell, particularly a plant or fungal cell is now a fairly routine technique.

In the particular case of a plant cell, the resulting transformed plant cell may then be used to regenerate a transformed plant in a manner known to persons skilled in the art.

Advantageously, any of several transformation methods may be used to introduce the gene or polynucleotide of the present invention into a suitable ancestor cell. Transformation methods include the use of liposomes, electroporation, chemicals that increase free DNA uptake, injection of the DNA directly into the (plant) cell, particle gun bombardment, transformation using viruses or pollen and microprojection. Methods may be selected from the calcium/polyethylene glycol method for protoplasts (11); electroporation of protoplasts (12); microinjection into (plant) material (13); DNA or RNA-coated particle bombardment (14) infection with (non-integrative) viruses and the like.

Transgenic plants, including transgenic crop plants, are preferably produced via Agrobacterium-mediated transformation (e.g. as described in WO 94/00977, WO97/48814; WO98/54961). Said methods are further described by way of example in B. Jenes et al., Techniques for Gene Transfer, in: Transgenic Plants, Vol. 1, Engineering and Utilization, eds. S. D. Kung and R. Wu, Academic Press (1993) 128-143 and in Potrykus Annu. Rev. Plant Physiol. Plant Molec. Biol. 42 (1991) 205-225. The nucleic acid or the chimeric genetic construct of the present invention is preferably cloned into a vector, which is suitable for transforming Agrobacterium tumefaciens, for example pBin19. Agrobacteria transformed by such a vector can then be used in known manner for the transformation of plants, such as described in for example, Hofgen and Willmitzer in Nucl. Acid Res. (1988) 16, 9877 or is known inter alia from F. F. White, Vectors for Gene Transfer in Higher Plants; in Transgenic Plants, Vol. 1, Engineering and Utilization, eds. S. D. Kung and R. Wu, Academic Press, 1993, pp. 15-38.

In the particular case of plants, generally after transformation, the plant cells or cell groupings are selected for the presence of one or more markers which are encoded by suitable plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants.

Alternatively, following DNA transfer and regeneration, putatively transformed host cells, including but not limited to plants or plant cells, may be evaluated, for instance using Southern analysis, for the presence of the gene of interest, copy number and/or genomic organisation. Alternatively or additionally, expression levels of the newly introduced DNA in said host cell, including but not limited to plant cells, may be undertaken using Northern and/or Western analysis, both techniques being well known to persons having ordinary skill in the art.

A whole organism may be regenerated from a single transformed or transfected cell, using methods known in the art. Plant tissue capable of subsequent clonal propagation, whether by organogenesis or embryogenesis, may be transformed with a genetic construct of the present invention and a whole plant regenerated therefrom. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem).

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The term “plant” as used herein encompasses whole plants, ancestors and progeny of the plants and plant parts, including seeds, shoots, stems, leaves, roots (including tubers), flowers, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen and microspores, again wherein each of the aforementioned comprises the gene/nucleic acid of interest. Plants that are particularly useful in the methods of the invention include in particular monocotyledonous and dicotyledonous plants including fodder or forage legumes, ornamental plants, food crops, trees or shrubs, such as rice, maize, wheat, barley, soybean, sunflower, canola, alfalfa, millet, barley, rapeseed, cotton, amaranth, artichoke, asparagus, broccoli, Brussels sprouts, cabbage, carrot, cauliflower, celery, collard greens, flax, kale, lentil, oilseed rape, okra, onion, potato, sugar beet, sugar cane, tomato, squash, or tea.

Furthermore, the characteristic of the transgenic cells or non-human organisms (plants) of the present invention to display tolerance to oxidative stress conditions may be combined with other approaches to confer abiotic or environmental stress tolerance, in particular oxidative stress tolerance, to said cell or organism. Thus, the approach of the present invention to confer tolerance to oxidative stress conditions to a host cell or (non-human) organism can be combined with known approaches, including the introduction of other stress tolerance genes. Combination of these approaches may have additive and/or synergistic effects in enhancing tolerance or resistance to oxidative and/or environmental stress.

Another embodiment of the invention provides host cells comprising a nucleic acid encoding a peptide of the present invention (as defined above) that increases the tolerance of a host cell or organism to oxidative stress conditions and/or increases the viability of said cell in oxidative stress conditions or in the presence of an agent inducing mitochondrial dysfunction. Preferred host cells are eukaryotic cells, including plant cells, fungal cells, yeast cells or mammal and human cells. The peptides of the present invention may also be produced by recombinant expression in prokaryotic and eukaryotic engineered cells other than plant cells, such as bacteria, fungi, or animal cells. Suitable expression systems are known to those skilled in the art.

The invention thus also extends to transgenic plants or eukaryotic microbial organisms, such as fungi or yeast, resistant to oxidative stress conditions and/or the presence of an agent resulting in mitochondrial dysfunction, wherein said plant or eukaryotic microbial organism has elevated levels of a peptide of the present invention compared to the corresponding wild type plant or eukaryotic microbial organism. Another embodiment of the present invention thus also provides a plant or an eukaryotic microbial organism obtainable by a method according to the present invention, wherein said plant or microbial organism has increased stress tolerance and has an altered level of a peptide of the present invention or a homologue thereof and/or has an altered expression of a nucleic acid encoding a peptide of the present invention or a homologue thereof.

Furthermore, a transgenic (non-human) organism for the purposes of the invention is thus understood as meaning that the nucleic acids used in the method of the invention (e.g. the chimeric genetic constructs) are not present in, or originating from, the genome of said organism, or are present in the genome of said organism but not at their natural locus in the genome of said organism, it being possible for the nucleic acids to be expressed homologously or heterologously. However, as mentioned, transgenic also means that, while the nucleic acids according to the invention or used in the inventive method are at their natural position in the genome of said organism, the sequence has been modified with regard to the natural sequence, and/or that the regulatory sequences of the natural sequences have been modified. Transgenic is preferably understood as meaning the expression of the nucleic acids according to the invention at an unnatural locus in the genome, i.e. homologous or, heterologous expression of the nucleic acids takes place.

In particular, the present invention provides plants or (non-human) organisms with increased tolerance to oxidative and/or abiotic stress, whereby said plant or (non-human) organism have increased expression of a nucleic acid encoding an oxidative stress tolerance increasing peptide comprising an amino acid sequence having at least 70%, preferably 80% or 90%, more preferably 95%, 96%, 97%, 98%, 99% or 100% sequence identity or similarity to the amino acid sequence as given in SEQ ID No. 1, SEQ ID No. 2, SEQ ID No. 3, SEQ ID No. 4, SEQ ID No. 5, SEQ ID No. 6 or SEQ ID No. 7, preferably as given in SEQ ID No. 1.

The present invention extends to any plant cell, plant or plant part or fungal or yeast cell obtained by any of the methods described herein, and to all plant parts, including harvestable parts of a plant, seeds and propagules thereof. The present invention also encompasses a plant or a part thereof comprising a plant cell transformed with a nucleic acid of the invention. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced in the parent by the methods according to the invention.

The methods of the present invention to create an eukaryotic (non-human) organism, preferably a plant, with enhanced tolerance to (oxidative) stress can also be combined with other genes or traits of interest, known in the art, for example: herbicide tolerance; insect resistance; virus resistance; improving the preserving of fruits; improvement of starch composition and/or production; altering lipid composition; the production of (bio)polymers; alteration of the flower colour, e.g., by manipulating the anthocyanin and flavonoid biosynthetic pathway; resistance to bacteria, insects and fungi; inducing maintaining male and/or female sterility; other abiotic stress resistance, (e.g. temperature stress).

As discussed above, a nucleic acid according to the invention will have the capacity to increase tolerance to oxidative stress in a host cell or organism transfected therewith, preferably an eukaryotic host cell or organism, such as a plant or fungal cell or organism. The oxidative stress tolerance inducing effect may also be obtained by applying the peptide of the invention (as defined above) directly to said host cell or organism.

Thus, in a different embodiment, the present invention relates to a protective composition comprising a peptide or peptidomimetic of the invention, a nucleic acid of the invention, a genetic construct or vector of the invention or the host cell of the invention. In a preferred embodiment, said composition comprises an effective amount of an isolated peptide comprising an amino acid sequence which is at least 70% or 80%, more preferably at least 90%, more preferably at least 95%, 97%, 99% or 100% identical to amino acid sequence SEQ ID No. 1; SEQ ID No. 2; SEQ ID No. 3; SEQ ID No. 4; SEQ ID No. 5; SEQ ID No. 6 or SEQ ID No. 7, most preferably to SEQ ID No. 1. Preferably, said composition further comprises a physiologically acceptable carrier and/or diluent. In a preferred embodiment, the composition is a plant-protective composition. The term “plant-protective composition” relates to compositions used in the prevention or treatment of diseases related to plants, particularly in the prevention or treatment of oxidative stress conditions. Formulations of plant-protective compositions comprise wettable powders (WPs), emulsifiable concentrates (ECs), or emulsifiable microemulsion concentrates. Microemulsion is a colloidal system which, in a first approach differs from a true emulsion in the dimension of its particles which are smaller by an order of magnitude than those of a true emulsion. According to the general definition, this system contains surface active agents and two immiscible liquids, one of them is usually water, though, in principle, it is also possible to prepare a water-free microemulsion by using another solvent. The surfactant may be the mixture of even 6 to 8 tensides and additionally, it may contain alcohols or amines of medium chain length as auxiliary surfactants (cosurfactants).

A “physiologically acceptable carrier” is a nontoxic solid, semisolid or liquid filler, diluent, encapsulating material or formulation auxiliary of any type. By “agronomically acceptable carrier” is meant a solid or liquid filler, diluent or encapsulating substance that can be safely used in topical or systemic administration of a peptide of the invention to a plant, plant seed or plant cell. Examples of suitable carriers are well known in the art and comprise water or organic solvents as liquids.

### Oxidative Stress Related Disorders

As mentioned, a peptide or nucleic acid of the present invention may be used to treat oxidative-stress related disorders both for veterinary and for human use. Oxidative stress may be responsible for initiating or otherwise causing disease. Alternatively, or additionally, the progression of the disease can be affected by any resultant oxidative stress. Particularly, oxidative stress and free radical damage is related to mitochondrial dysfunction.

Hence the phrase “oxidative stress related disorder” as used herein, refers to a disease or medical condition (including syndromes) wherein the onset or progression thereof is promoted by oxidative stress, in particular wherein the healthy function of one or more organelles, non-organelle subcellular structures, cell, cell types, tissues, tissue types, organs, or organ systems, particularly the mitochondria, is impaired by the action of oxidizing agents, particularly ROS. The action of oxidizing agents need not be the only route by which impairment of healthy function occurs in the course of a disease for the disease to be an oxidative stress disease.

Since oxidative stress is believed to be responsible for the pathogenesis of many neurological, heart, malignant and age-associated diseases, the present invention contemplates all such diseases including for example: a central nervous system (CNS) neurodegenerative disease, including but not limited to Parkinson's disease, Alzheimer's disease, multiple sclerosis, amyotrophic lateral sclerosis, or Huntington's disease; stroke; atherosclerosis; myocardial ischemia; myocardial reperfusion; autoimmune diseases; cancer; cardiovascular disease or diabetes or a complication of diabetes; circulatory impairment; retinopathy; blindness; kidney disease; pancreas disease; neuropathy; gum disease; cataracts; skin disease; skin damage by flame, heat, radiation (including ultraviolet light radiation). In one embodiment, the oxidative stress disease is senescence. “Senescence,” as used herein, refers to one or more of a decrease in the overall health of a mammal, a decrease in the overall fitness of a mammal, or a decrease in the overall quality of life of a mammal, wherein such decrease is generally attributed to the aging process. Ameliorating senescence may lead to maintenance of a particular level of systemic well-being to a later point in the mammal's life, or may lead to at least a partial increase in the expected lifespan of the organism, such as a mammal.

Preferably, said oxidative stress related disease is a mitochondrial dysfunction related disorder or syndrome. Mitochondrial dysfunction relates to abnormalities in mitochondria and diseases and conditions associated with or involving decreased mitochondrial function. Conditions and diseases including various neurodegenerative diseases, including but not limited to Parkinson's disease, Alzheimer's disease and mitochondrial encephalopathies, as well as normal aging, complications of diabetes, and age-related macular degeneration are but a few examples of such diseases related to mitochondrial dysfunction. Other mitochondrial dysfunction diseases and syndrome include, the non-alcoholic fatty liver disease (NAFLD), the metabolic syndrome and disorders related to (inborn) errors in (mitochondrial) respiratory chain complexes, such as Wilson's Disease.

Thus, yet another preferred embodiment of the present invention relates to a peptide or peptidomimetic of the present invention for use in preventing, treating, ameliorating or diagnosing an oxidative stress-related disorder, particularly a mitochondrial dysfunction related disorder. Thus, the present invention provides a method of treating an oxidative stress-related disorder, preferably a mitochondrial dysfunction related disorder, in a subject in need thereof, the method comprising administering to the subject a therapeutically effective amount of said peptide or peptidomimetic of the present invention, thereby treating or ameliorating the oxidative stress-related or mitochondrial dysfunction related disorder. Alternatively, the present invention also relates to the use of a peptide or nucleic acid of the present invention for the manufacture of a medicament for the prevention and/or treatment of oxidative stress related disorders and to the use of said peptide or nucleic acid for the screening of materials for their therapeutic activity.

By “ameliorating” a disease or disorder is meant improving the condition of an organism suffering or at risk of suffering from the disease or disorder. Ameliorating can comprise one or more of the following: a reduction in the severity of a symptom of the disease, a reduction in the extent of a symptom of the disease, a reduction in the number of symptoms of the disease, a reduction in the number of disease agents, a reduction in the spread of a symptom of the disease, a delay in the onset of a symptom of the disease, a delay in disease onset, or a reduction in the time between onset of the disease and remission of the disease, among others apparent to the skilled artisan having the benefit of the present invention. To the extent that the foregoing examples of ameliorating a disease are defined in relative terms, the proper comparison is to the disease or symptoms thereof when no composition or material is administered to ameliorate it and no method is performed to ameliorate it. The terms “preventing” (herein meaning “to stop a disease from onsetting”) and “treating” (herein meaning “to improve the condition of an organism, such as a mammal, suffering from a disease”) are both within the scope of “ameliorating,” as used herein.

A peptide or peptidomimetic of the present invention may be provided per se or as part of a pharmaceutical composition, where it may be formulated with conventional carriers and excipients, which will be selected in accord with ordinary practice. Tablets will contain excipients, glidants, fillers, binders and the like. Aqueous formulations are prepared in sterile form, and when intended for delivery by other than oral administration generally will be isotonic. Formulations optionally contain excipients such as those set forth in the “Handbook of Pharmaceutical Excipients” (1986). As used herein a “pharmaceutical composition” refers to a preparation of one or more of the active ingredients described herein, i.e. a peptide or peptidomimetic of the present invention, with other chemical components such as pharmaceutically acceptable carriers and excipients. The purpose of a pharmaceutical composition is to facilitate administration of a compound to an organism. Thus, the present invention also relates to a composition comprising a) a peptide comprising an amino acid sequence which is at least 70%, more preferably at least 80%, more preferably at least 90%, more preferably at least 95%, 97%, 99% or 100% identical to an amino acid sequence selected from the amino acid sequences SEQ ID No. 1 to SEQ ID No. 575, preferably to an amino acid sequence selected from SEQ ID No:1, SEQ ID No. 2, SEQ ID No. 3, SEQ ID No. 4, SEQ ID No. 5, SEQ ID No. 6 or SEQ ID No. 7, more preferably to amino acid sequence SEQ ID No. 1, or a nucleic acid encoding for said peptide and b) one or more pharmaceutically acceptable compounds, carriers and/or adjuvants. The compositions of this invention can suitably be used as concentrates, emulsions, solutions, granulates, dusts, sprays, aerosols, suspensions, ointments, creams, tablets, pellets or powders.

The phrase “pharmaceutically acceptable carrier” as used herein refers to any material, substance, or diluent that does not cause significant irritation to an organism and does not abrogate the biological activity and properties of the administered compound with which the active ingredient is formulated in order to facilitate its application or dissemination to the locus to be treated, for instance by dissolving, dispersing or diffusing the said composition, and/or to facilitate its storage, transport or handling without impairing its effectiveness. An adjuvant is included under these phrases. The term “excipient” as used herein refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols. The term “active ingredient” refers to the oxidative stress tolerance inducing peptide(s) of the present invention accountable for the biological effect.

Techniques for formulation and administration of drugs may be found in “Remington's Pharmaceutical Sciences,” Mack Publishing Co., Easton, Pa., latest edition (herein incorporated by reference). Suitable pharmaceutical carriers for use in the said pharmaceutical compositions and their formulation are well known to those skilled in the art, and there is no particular restriction to their selection within the present invention. They may also include additives such as wetting agents, dispersing agents, stickers, adhesives, emulsifying agents, solvents, coatings, antibacterial and antifungal agents (for example phenol, sorbic acid, chlorobutanol), isotonic agents (such as sugars or sodium chloride) and the like, provided the same are consistent with pharmaceutical practice, i.e. carriers and additives which do not create permanent damage to mammals. The pharmaceutical compositions of the present invention may be prepared in any known manner, for instance by homogeneously mixing, coating and/or grinding the active ingredients, in a one-step or multi-steps procedure, with the selected carrier material and, where appropriate, the other additives.

The oxidative stress tolerance inducing agents of the present invention and pharmaceutical compositions thereof may be administered by any route appropriate to the condition to be treated. Suitable routes of administration may, for example, include oral, rectal, nasal, topical (including ocular, buccal and sublingual), vaginal and parenteral (including subcutaneous, intramuscular, intravenous, intradermal, intrathecal and epidural). The preferred route of administration may vary with for example the condition of the recipient.

Alternately, one may administer the pharmaceutical composition in a local rather than systemic manner, for example, via injection of the pharmaceutical composition directly into a tissue region of a patient.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

The formulations may be optionally applied as a topical ointment or cream containing the active ingredient(s) (e.g. sun screen), such as e.g. when treating (prophylactically or therapeutically) oxidative damage to the skin of a patient. When formulated in an ointment, the active ingredients may be employed with either a paraffinic or a water-miscible ointment base. Alternatively, the active ingredients may be formulated in a cream with an oil-in-water cream base. If desired, the aqueous phase of the cream base may include, for example, at least 30% w/w of a polyhydric alcohol, i.e. an alcohol having two or more hydroxyll groups such as propylene glycol, butane 1,3-diol, mannitol, sorbitol, glycerol and polyethylene glycol (including PEG400) and mixtures thereof. The topical formulations may desirably include a compound which enhances absorption or penetration of the active ingredient through the skin or other affected areas. Examples of such dermal penetration enhancers include dimethylsulfoxide and related analogs. The oily phase of said emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Optionally, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuos infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous sterile solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions. The parenteral solutions may further contain anti-oxidants, buffers, bacteriostats and solutes which render the formulation isotonic with the blood of the intended recipient.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients, i.e. the oxidative stress tolerance inducing peptides of the present invention, effective to prevent, alleviate or ameliorate symptoms of a disorder (e.g., Parkinson's Disease) or prolong the survival of the subject being treated.

Determination of a therapeutically effective amount is well within the capability of those skilled in the art. For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.l—incorporated by reference herein).

Dosage amount and interval may be adjusted individually to tissue or blood levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

TABLE 1
List of oxidative stress inhibiting peptides deduced from the different open
reading frames of the OSIP encoding regions (based on tiling array data)

| PEPTIDE CODE | AMINO ACID CODE | SEQ ID NO |
|---|---|---|
| >SIP108_4\|PQ4\|−\|3 | MLCVLQGLRE- | SEQ ID No. 1 |
| >SIP14_1\|PQ4\|+\|2 | MIIINNDNYLLLFYNNN- | SEQ ID No. 2 |
| >SIP14_2\|PQ4\|−\|3 | MIIIYYCFIIIIN- | SEQ ID No. 3 |
| >SIP11_0\|PQ4\|+\|3 | MLMYRMRSGAN- | SEQ ID No. 4 |
| >SIP163_3\|PQ4\|−\|3 | MGLNEDSVFRSIKPFKSP- | SEQ ID No. 5 |
| >SIP152_2\|PQ4\|+\|3 | MNVLARAPRLRHQLQNLTQDRRKTQMQMKGQRVRTTSLQ- | SEQ ID No. 6 |
| >SIP37_1\|PQ4\|−\|1 | MAREEKEQSVYDIYTFASLLL- | SEQ ID No. 7 |
| >SIP74_2\|PQ4\|+\|3 | MHCLLSLLEMVPSNLLSALLNLN- | SEQ ID No. 8 |
| >SIP74_3\|PQ4\|−\|1 | MSVEICRRYYLPPPNNCTYTTPQTSI- | SEQ ID No. 9 |
| >SIP74_4\|PQ4\|−\|2 | MICYKNLARQNIN- | SEQ ID No. 10 |
| >SIP74_5\|PQ4\|−\|3 | MSSYQSIYEC- | SEQ ID No. 11 |
| >SIP133_1\|PQ4\|+\|3 | MIHIHINWRLIRIG- | SEQ ID No. 12 |
| >SIP133_2\|PQ4\|−\|1 | MHIYALANSNQSPINVYMYHSHRP- | SEQ ID No. 13 |
| >SIP133_3\|PQ4\|−\|2 | MCICIIHIGPRLILTILQYVF- | SEQ ID No. 14 |
| >SIP89_0\|PQ4\|+\|1 | MSVKKRALESKNNGSPKSSHYNRCLSFSFLESSTGEKK | SEQ ID No. 15 |
| | KPSSLNRMDSKILMEIVKWAKRVAAYARQLSSRKQD- | |
| >SIP89_1\|PQ4\|+\|1 | MKLVQADPFSDCF- | SEQ ID No. 16 |
| >SIP89_2\|PQ4\|+\|2 | MEAPSRVIIIDACHSRSWSHQRERRRNHHR- | SEQ ID No. 17 |
| >SIP89_3\|PQ4\|+\|2 | MLVNLARENKIKT- | SEQ ID No. 18 |
| >SIP89_4\|PQ4\|+\|2 | MKKAYGKIDMHSFYIDLGPHSFNHS- | SEQ ID No. 19 |
| >SIP89_5\|PQ4\|+\|3 | MLVILVLGVINGREEETIIVEPYGFQNTQGRDR- | SEQ ID No. 20 |
| >SIP89_6\|PQ4\|+\|3 | MGRLICTHFTLI- | SEQ ID No. 21 |
| >SIP89_7\|PQ4\|+\|3 | MLRSHETCTSRSI- | SEQ ID No. 22 |
| >SIP89_8\|PQ4\|−\|1 | MSAYQSSHKLSSSMS- | SEQ ID No. 23 |
| >SIP5_0\|PQ4\|+\|1 | MNKTQSHLLFSLSTCLNSYVNTKLPSILVNSKLSINT- | SEQ ID No. 24 |
| >SIP5_3\|PQ4\|+\|2 | MNHLQISLIS- | SEQ ID No. 25 |
| >SIP5_4\|PQ4\|+\|3 | MPKLICEHQTPIHSRQFKALYQYITSITLPMR- | SEQ ID No. 26 |
| >SIP5_5\|PQ4\|−\|1 | MGVWCSHMSLGMLIS- | SEQ ID No. 27 |
| >SIP5_6\|PQ4\|−\|2 | MGRGKVWVVEQSRRKI- | SEQ ID No. 28 |
| >SIP5_7\|PQ4\|−\|3 | MDGSLVFTYEFRHVDKLKSKWD- | SEQ ID No. 29 |
| >SIP32_0\|PQ4\|+\|1 | MVKWSGDVTTDCILCQGNLETREHLFFDCGYTSAVWA | SEQ ID No. 30 |
| | ALVKGILKSRYTSNVVTSIMDHLAHAQPHRVDHFLVRYAF | |
| | QATLYTVWRERNGRRHGETLNTASQLVGWIDKQIRNQL | |
| | SSIKLKGDRRYDDALQLWFSTRV- | |
| >SIP32_2\|PQ4\|+\|2 | MLSHTAWTTSSFAMPSKRLYTLCGGKEMVDDTVRPSTRPLN- | SEQ ID No. 31 |
| >SIP32_4\|PQ4\|+\|3 | MVGRRYNGLHSLSR- | SEQ ID No. 32 |
| >SIP32_5\|PQ4\|+\|3 | MDRQANPKSAVLNQAQGRSTI- | SEQ ID No. 33 |
| >SIP32_6\|PQ4\|−\|2 | MSEWHYRGPVGSISRFENSLYKRCPNC- | SEQ ID No. 34 |
| >SIP32_7\|PQ4\|−\|3 | MQRELKEFKLKIEFEKKETKL- | SEQ ID No. 35 |
| >SIP108_0\|PQ4\|+\|1 | MDNRDKSCFANKHAVAGKLIGFRTPAYSLKP- | SEQ ID No. 36 |
| >SIP108_2\|PQ4\|−\|2 | MCASRFKGVGGCSKTDQFTCNCMFICKT- | SEQ ID No. 37 |
| >SIP108_3\|PQ4\|−\|3 | MVTKETQAEGEKVCVIIIL- | SEQ ID No. 38 |
| >SIP75_1\|PQ4\|+\|2 | MLCCWDFDRSLPLILL- | SEQ ID No. 39 |
| >SIP84_0\|PQ4\|+\|1 | MGSLAEEGGDLLLPRGGVSGGAKTGEGLCSGDKGWS | SEQ ID No. 40 |
| | VETSSRTSGIIPMLKHIIELRT- | |
| >SIP84_3\|PQ4\|−\|3 | MVRCYRTSRTRCSIDRWGRVLLQLGDHIARPRIMIGEVKR- | SEQ ID No. 41 |
| >SIP84_5\|PQ4\|−\|1 | MWSPSWRSTLPQRSMLQRVRLVR- | SEQ ID No. 42 |
| >SIP84_6\|PQ4\|−\|2 | MPPQKENHRTLNKMKTNLFLFLIFSLLLSLSSAEQCGRQ | SEQ ID No. 43 |
| | AGGALCPNGLCCSEFGWCGNTEPYCKQPGCQSQCTP | |
| | GGTPPGPTGDLSGIISSSQFDDMLKHRNDAACPARGFY | |
| | TYNAFITAAKSFPGFGTTGDTATRKKEVAAFFGQTSHET | |
| | TGTQNSLTI | |
| >SIP84_7\|PQ4\|−\|3 | MICLSIGMMPLVLLEVSTLTTPLSPLQSPSPVLAPPETPP | SEQ ID No. 44 |
| | RGRRRSPPSSARLPMKLQVPKTH- | |
| >SIP129_0\|PQ4\|+\|1 | MSTTKNFFRLYQNQHPNTSSSLLFYLALIDAKGLNRRESKTP- | SEQ ID No. 45 |
| >SIP129_2\|PQ4\|−\|2 | MLYAQALILCFI- | SEQ ID No. 46 |
| >SIP129_3\|PQ4\|−\|3 | MIMCSLFASVFDCFVPKSDSKISSTDESDLKVLSSKKPK | SEQ ID No. 47 |
| | SKSPRAPIMVSYFPAGSNLSRL- | |
| >SIP129_4\|PQ4\|−\|3 | MKYWDVDFDINERSFWL- | SEQ ID No. 48 |
| >SIP148_0\|PQ4\|+\|1 | MLYKNSNDSM- | SEQ ID No. 49 |
| >SIP148_2\|PQ4\|+\|3 | MDRVKSNICSHIPLIGCVMDVLFLLFTSDSSSDAGKEIVD | SEQ ID No. 50 |
| | TGNLEAQKNFRQLQCIKLG- | |
| >SIP148_4\|PQ4\|−\|2 | MTHPIRGICEQILDFTRSIKL- | SEQ ID No. 51 |
| >SIP163_0\|PQ4\|+\|1 | MPQLCLDLMKSLKL- | SEQ ID No. 52 |
| >SIP163_1\|PQ4\|+\|1 | MITVLTFTLNFQLSSLKGRRNYHGLLKGFMLRNTLSSFK | SEQ ID No. 53 |
| | PILYDPNNIGQLFAYFPLSVLEKENEVD- | |
| >SIP163_2\|PQ4\|−\|2 | MIIPSSLKTRKLKVESKS- | SEQ ID No. 54 |
| >SIP75_2\|PQ4\|+\|2 | MSRRMILTQYW- | SEQ ID No. 55 |
| >SIP147_0\|PQ4\|+\|1 | MSLLLNLFMGLFLCGILKEVHKNEGEEEELVETELVEME | SEQ ID No. 56 |
| | LVETTSFYLITILKILFH- | |
| >SIP147_1\|PQ4\|+\|2 | MKPYMINNFS- | SEQ ID No. 57 |
| >SIP147_3\|PQ4\|−\|2 | MSSPPIPSPLIPSPPIPPPPPRFYVPPSKSRRGKGP- | SEQ ID No. 58 |
| >SIP147_4\|PQ4\|−\|3 | MVANYLMKQYLQYCYQVKRCRLHQFHLH- | SEQ ID No. 59 |
| >SIP147_5\|PQ4\|−\|3 | MYLLQNPAEEKAHKQIE- | SEQ ID No. 60 |
| >SIP152_1\|PQ4\|+\|2 | MQENKARVTTEKRSVCRTPPINTENRFDCLMIC- | SEQ ID No. 61 |
| >SIP75_3\|PQ4\|+\|3 | MHLRKRRRLLSQKRTKVV- | SEQ ID No. 62 |
| >SIP152_3\|PQ4\|−\|1 | MARHSLLSPPYDSPLSLYIFSF- | SEQ ID No. 63 |
| >SIP152_4\|PQ4\|−\|2 | MTVLFHCTYSLFDDK- | SEQ ID No. 64 |
| >SIP152_5\|PQ4\|−\|3 | MLSRSSNNQICSPC- | SEQ ID No. 65 |
| >SIP152_6\|PQ4\|−\|3 | MKKICWASQLS- | SEQ ID No. 66 |
| >SIP87_1\|PQ4\|+\|2 | MDPAMEVDMVKGMEADTWEIITVTHPKSFLLLIVS- | SEQ ID No. 67 |
| >SIP87_2\|PQ4\|+\|3 | MIIMCSMFCRYIYIHACI- | SEQ ID No. 68 |
| >SIP87_3\|PQ4\|−\|1 | MYIYVTTKHRTHYYHKFRIMKQ- | SEQ ID No. 69 |
| >SIP8_1\|PQ4\|+\|1 | MFCKFAIFHFYA- | SEQ ID No. 70 |
| >SIP8_2\|PQ4\|+\|2 | MEETLYTQHQK- | SEQ ID No. 71 |
| >SIP8_3\|PQ4\|+\|2 | MPENQHKSGE- | SEQ ID No. 72 |
| >SIP8_6\|PQ4\|−\|3 | MEDCELAEHDLVIETAVSSAGKLNPFLMFSYFWCCV- | SEQ ID No. 73 |
| >SIP8_7\|PQ4\|−\|3 | MRFLYNTLPGFQIDFFSSV- | SEQ ID No. 74 |
| >SIP58_2\|PQ4\|+\|2 | MCCQPLPLAIPLRTAVKPSRIKERQSKGVRSAT- | SEQ ID No. 75 |
| >SIP58_3\|PQ4\|+\|2 | MMFLQVNRSMLKKADSTIQRQSLANTETLAT- | SEQ ID No. 76 |
| >SIP58_4\|PQ4\|−\|1 | MISILSFRTSSSYYRQSFHKDWFLAAHVYQTIWELGILKL | SEQ ID No. 77 |
| | LGSQY- | |
| >SIP58_5\|PQ4\|−\|1 | MGLLGEWGNTLELGTILFLIISYELLLEFT- | SEQ ID No. 78 |
| >SIP58_6\|PQ4\|−\|2 | MSSSWNLLSFQS- | SEQ ID No. 79 |
| >SIP145_1\|PQ4\|+\|2 | MNELQRLYLKT- | SEQ ID No. 80 |
| >SIP145_2\|PQ4\|+\|2 | MIYESQDTTDKLIPEHMSNRLTMTGSKDIK- | SEQ ID No. 81 |
| >SIP145_4\|PQ4\|+\|3 | MIYESQDTTDKLISKHMSNRLKQT- | SEQ ID No. 82 |
| >SIP145_5\|PQ4\|−\|2 | MCLDINLSWSWDS- | SEQ ID No. 83 |
| >SIP145_6\|PQ4\|−\|2 | MIVSKYLQPI- | SEQ ID No. 84 |
| >SIP145_7\|PQ4\|−\|3 | MSLDPVIVSLLLMCSGISLSWSWDS- | SEQ ID No. 85 |
| >SIP43_1\|PQ4\|+\|3 | MRIMWKFIIVMLCFIALVGSRGTSATSRLRMKKEDIGRRF | SEQ ID No. 86 |
| | ALQNKLQRGPVPPSQPSPCHNKLNPLSHSQVYSSHTYVTCP- | |
| >SIP43_2\|PQ4\|−\|1 | MIKWLNNTKSCIIYVQKVLQFRLFRVWRSRIVDTYMYEK | SEQ ID No. 87 |
| | RDNK- | |
| >SIP43_3\|PQ4\|−\|2 | MSKRYYNFDSFEFGGVESWIHICMRRETINKRMWLIKLQGQ | SEQ ID No. 88 |
| | VT- | |
| >SIP43_4\|PQ4\|−\|2 | MNFHMILMITRPCNAGMNGLWD- | SEQ ID No. 89 |
| >SIP43_5\|PQ4\|−\|3 | MEKAVKEELDLVGAYSAKQTFYLYLPSSFSALTLPMFLLI | SEQ ID No. 90 |
| | PPKL- | |
| >SIP65_2\|PQ4\|+\|3 | MRDQFNLQISAW- | SEQ ID No. 91 |
| >SIP65_3\|PQ4\|+\|3 | MNQIQLIYFNFLQPQCTIYLIFLFTKVFIYNTKNI- | SEQ ID No. 92 |
| >SIP65_5\|PQ4\|−\|1 | MGHHRILRKTHTSYIFRVIYKNFGEQKN- | SEQ ID No. 93 |
| >SIP6_3113Q4\|−\|2 | MIHHLYLARNPRISDTTSLITPILKC- | SEQ ID No. 94 |
| >SIP144_1\|PQ4\|−\|3 | MQNQIEKRCQFY- | SEQ ID No. 95 |
| >SIP144_2\|PQ4\|−\|3 | MNNELQQLYLKTKSKSHKQDIISFFLIQ- | SEQ ID No. 96 |
| >SIP78_0\|PQ4\|−\|1 | MSPRTQVTNTATGTQATVRIVDQCSNGGLDLEEGVFRQ | SEQ ID No. 97 |
| | LDINSQGNARGHLIVNYEFVNC- | |
| >SIP78_1\|PQ4\|−\|2 | MLFSYTNLLNVCICPLERR- | SEQ ID No. 98 |
| >SIP78_2\|PQ4\|−\|3 | MCAYVPSNAGDEYCDRNASDCENRGSV- | SEQ ID No. 99 |
| >SIP149_1\|PQ4\|+\|1 | MSCSDENLLTLDGVFNCVL- | SEQ ID No. 100 |
| >SIP149_2\|PQ4\|+\|2 | MTISYIFFLLKTAI- | SEQ ID No. 101 |
| >SIP149_3\|PQ4\|+\|3 | MTADLGKSSSFSSRRPGSLDVLFRRESAHIGRRLQLCS | SEQ ID No. 102 |
| | MIRY- | |
| >SIP149_5\|PQ4\|−\|1 | MCNRIFPDICP- | SEQ ID No. 103 |
| >SIP149_6\|PQ4\|−\|1 | MMMIFQDPLSSHHYIAVLRRKKIYDIVIVEEILVRS- | SEQ ID No. 104 |
| >SIP149_7\|PQ4\|−\|2 | MILSLSRRYLFDREREKY- | SEQ ID No. 105 |
| >SIP155_0\|PQ4\|+\|1 | MKKLRQRVAKKIKQDICGDNSIVFFFLVILTTFLILNYLIN | SEQ ID No. 106 |
| | SL- | |
| >SIP155_2\|PQ4\|−\|3 | MWSISPRKKKQYCCHHKCLVLFSLPLFA- | SEQ ID No. 107 |
| >SIP162_0\|PQ4\|+\|1 | MTWSNVIQLSLELKQPATSGT- | SEQ ID No. 108 |
| >SIP162_1\|PQ4\|+\|1 | MPLPVQALSLQVWD- | SEQ ID No. 109 |
| >SIP162_2\|PQ4\|+\|1 | MWCKHSSLLFLR- | SEQ ID No. 110 |
| >SIP162_4\|PQ4\|−\|2 | MDQSQTWRDKACTGKGIVD- | SEQ ID No. 111 |
| >SIP162_5\|PQ4\|−\|2 | MRNSQVPEVAGCFNSSESWMTFDQVICLDVANGNASS | SEQ ID No. 112 |
| | FFHHYCSR- | |
| >SIP162_6\|PQ4\|−\|3 | MIFTLETGGRSVYTTSG- | SEQ ID No. 113 |
| >SIP134_0\|PQ4\|+\|1 | MGYGRSLIWLAEDTFGEISRRCVREVNDRYKQSFACLASDM- | SEQ ID No. 114 |
| >SIP134_1\|PQ4\|+\|2 | MTDINKVSPVWLLICRKLRTRKIVSSFS- | SEQ ID No. 115 |
| >SIP134_3\|PQ4\|−\|2 | MKLPNSDEFGQDLRKRRNNFSCSQLSTYQKPNRRNFVYIGH- | SEQ ID No. 116 |
| >SIP134_4\|PQ4\|−\|3 | MKFKYMNTKLKNE- | SEQ ID No. 117 |
| >SIP134_5\|PQ4\|−\|3 | MNSDKIYENEETIFLVLNFLHIRSQTGETLFISVIDFSDASP- | SEQ ID No. 118 |
| >SIP26_0\|PQ4\|+\|3 | MQQRVNCGHHLDLL- | SEQ ID No. 119 |
| >SIP26_2\|PQ4\|−\|3 | MVNHLQQIQMVTAIDSLLHRLYQGRQRLN- | SEQ ID No. 120 |
| >SIPQ_0\|PQ4\|+\|1 | MKERSKTQLN- | SEQ ID No. 121 |
| >SIPQ_1\|PQ4\|+\|3 | MGYRNRSYLVKSLPF- | SEQ ID No. 122 |
| >SIP99_0\|PQ41\|−\|1 | MRSRRIKKTRRIKRTRRIN- | SEQ ID No. 123 |
| >SIP99_1\|PQ4\|+\|2 | MFFEQRLGFHLLAGLVWLVNPSRF- | SEQ ID No. 124 |
| >SIP102_1\|PQ4\|+\|1 | MLSNDANRETGKSLYLKG- | SEQ ID No. 125 |
| >SIP102_3\|PQ4\|+\|2 | MMQTVKLESLYISRDKTPELALLWL- | SEQ ID No. 126 |
| >SIP120_0\|PQ4\|−\|2 | MPITSSLPILGSEAGKDGQEEDNTAASGGIMAAGTPVTH | SEQ ID No. 127 |
| | PKGKPV- | |
| >SIP31_0\|PQ4\|+\|1 | MSFLAENVESRFLAKKC- | SEQ ID No. 128 |
| >SIP31_1\|PQ4\|+\|1 | MLNCFLAGKY- | SEQ ID No. 129 |
| >SIP31_2\|PQ4\|+\|1 | MNWFGGKMLNCVLAGKY- | SEQ ID No. 130 |
| >SIP31_4\|PQ4\|+\|2 | MLIWFGEKMLNCVLAGK- | SEQ ID No. 131 |
| >SIP31_5\|PQ4\|+\|3 | MFNWFGGKC- | SEQ ID No. 132 |
| >SIP31_6\|PQ4\|+\|3 | MLNCVLARKY- | SEQ ID No. 133 |
| >SIP31_7\|PQ4\|+\|3 | MLNCVLAGKC- | SEQ ID No. 134 |
| >SIP103_2\|PQ4\|−\|1 | MSLIIQICFSLSERHLVVAYIHF- | SEQ ID No. 135 |
| >SIP103_4\|PQ4\|−\|3 | MALNNYRLQIPLKHLPIPRHKLRNELNNTNLLLLE- | SEQ ID No. 136 |
| >SIP44_0\|PQ4\|+\|2 | MMIIHLQVTQHSEKDRLRKNHTYKLFCKNIF- | SEQ ID No. 137 |
| >SIP44_3\|PQ4\|−\|1 | MYDFFSAYLFHYVV- | SEQ ID No. 138 |
| >SIP44_4\|PQ4\|−\|2 | MICRLRRLGREWSLVKIDWLKLRMFLRAISNYFCRC- | SEQ ID No. 139 |
| >SIP44_5\|PQ4\|−\|2 | MIFSQPIFFTMLCNL- | SEQ ID No. 140 |
| >SIP79_1\|PQ4\|+\|2 | MVSCSNVRCGLSGCFGRKRT- | SEQ ID No. 141 |
| >SIP79_3\|PQ4\|−\|1 | MSRLSLCVFVLLCAFAAKAAAQSAPNV- | SEQ ID No. 142 |
| >SIP79_5\|PQ4\|−\|3 | MCVCVAMCVCGQSSRSIRT- | SEQ ID No. 143 |
| >SIP106_0\|PQ4\|+\|2 | MMIASQAVLHSQLQATWLQQSQLKQN- | SEQ ID No. 144 |
| >SIP106_2\|PQ4\|−\|1 | MGLDGVTNVDFDELGVDVSKLLLSLKWFWLLD- | SEQ ID No. 145 |
| >SIP106_3\|PQ4\|−\|2 | MWILMNWVLTCRNCC- | SEQ ID No. 146 |
| >SIP106_4\|PQ4\|−\|3 | MNGSRWSNECGF- | SEQ ID No. 147 |
| >SIP106_5\|PQ4\|−\|3 | MVLVARLRLRAQARSCWC- | SEQ ID No. 148 |
| >SIP22_1\|PQ4\|+\|3 | MHGTDVTFRKDARSGCDL- | SEQ ID No. 149 |
| >SIP22_2\|PQ4\|−\|1 | MDASRSITSLPGTLPKGHIRSLHPSERSHLFHASFGDAT | SEQ ID No. 150 |
| | VRLYPLDLTPFGRDTSSPVSYQSTSSSVS- | |
| >SIP22_3\|PQ4\|−\|2 | MLPEASHPFLAPSLKVTSAPCILPKGHICSMHLLAMQPF | SEQ ID No. 151 |
| | VCTRLTLHLLAETPLHL- | |
| >SIP72_0\|PQ4\|+\|1 | MAELLQQVRY- | SEQ ID No. 152 |
| >SIP72_2\|PQ4\|+\|2 | MTLTCISQLLSS- | SEQ ID No. 153 |
| >SIP72_3\|PQ4\|−\|2 | MSVLVADLLEQFRHHLVELQISMLSYKLLMRFSLRILL- | SEQ ID No. 154 |
| >SIP36_1\|PQ4\|+\|2 | MILKCWSSRFLRVSPYQNAHSLSLG- | SEQ ID No. 155 |
| >SIP36_4\|PQ4\|−\|2 | MRILIWTHSQEPGTPAL- | SEQ ID No. 156 |
| >SIP153_2\|PQ4\|−\|3 | MAFFTNHFTIVLLYQFIFLIRTINFVIIMYEPKNPYSVYMKV | SEQ ID No. 157 |
| | SIWQLLI- | |
| >SIP17_1\|PQ4\|−\|2 | MSTKVNGGGDE- | SEQ ID No. 158 |
| >SIP17_2\|PQ4\|−\|2 | MDMMWNTEAWFTDLVILNTEEAWLRI- | SEQ ID No. 159 |
| >SIP17_3\|PQ4\|−\|3 | MNREEAVVLWI- | SEQ ID No. 160 |
| >SIP92_0\|PQ4\|−\|1 | MGIRYLNGPKRTRPTSCKPNFPSDK- | SEQ ID No. 161 |
| >SIP92_1\|PQ4\|−\|2 | MGQKEPGPPAVNLISLQTNEGRDGGGCCTEMTRGKRP | SEQ ID No. 162 |
| | SPVKERSTFAGFGREVRQRRRTRGS- | |
| >SIP92_2\|PQ4\|−\|3 | MKVVMVAAVVRR- | SEQ ID No. 163 |
| >SIP45_0\|PQ4\|−\|2 | MVFLRAISNYL- | SEQ ID No. 164 |
| >SIP45_1\|PQ4\|−\|3 | MICRPRRQGRD- | SEQ ID No. 165 |
| >SIP109_1\|PQ4\|−\|1 | MNMLLKSQRYMHYP- | SEQ ID No. 166 |
| >SIP109_2\|PQ4\|−\|3 | MILCEYIYEHALKVTKIYALPVDAPVTVGFVF- | SEQ ID No. 167 |
| >SIP76_0\|PQ4\|+\|3 | MFLSVLEFGS- | SEQ ID No. 168 |
| >SIP76_2\|PQ4\|−\|1 | MYLWGMQEAMMNDIVPSMMQ- | SEQ ID No. 169 |
| >SIP76_3\|PQ4\|−\|2 | MERCTCGACKKR- | SEQ ID No. 170 |
| >SIP76_4\|PQ4\|−\|3 | MCSHWKREWRDVLVGHARSDDE- | SEQ ID No. 171 |
| >SIP165_0\|PQ4\|+\|1 | MNHLNRPKVLTQGPLSKIFNSSNRYSLL- | SEQ ID No. 172 |
| >SIP165_2\|PQ4\|−\|1 | MSGYSYDCTGLFPVLL- | SEQ ID No. 173 |
| >SIP165_3\|PQ4\|−\|2 | MIARVCSQSYCEEELPINIQRHVCWKVFAVQAA- | SEQ ID No. 174 |
| >SIP19_1\|PQ4\|+\|1 | MSTSSTWNHMQHAIKHN- | SEQ ID No. 175 |
| >SIP19_2\|PQ4\|+\|2 | MRKYNNIESITSSNQ- | SEQ ID No. 176 |
| >SIP19_3\|PQ4\|+\|3 | MEPHATRNQT- | SEQ ID No. 177 |
| >SIP19_4\|PQ4\|−\|2 | MFDCVLHWPCTRRTHGNQLDCLFY- | SEQ ID No. 178 |
| >SIP19_6\|PQ4\|−\|3 | MWFHVLDVLMVTSLIVYFIDCLMLCFQYCYTSACCLLFW | SEQ ID No. 179 |
| | VLGWEIIVC- | |
| >SIP104_1\|PQ4\|−\|1 | MQQRLMMNLLDQWELVA- | SEQ ID No. 180 |
| >SIP104_2\|PQ4\|−\|2 | MHKLTSLHNPRRNRSWRRRLWRRGLRIMDLRVSNSW | SEQ ID No. 181 |
| | GNGFWRRDISLRRHFRRSRPNKRRCNRG- | |
| >SIP104_3\|PQ4\|−\|2 | MLSFGFRQDLFDTTRLSKNGKK- | SEQ ID No. 182 |
| >SIP88_1\|PQ4\|+\|2 | MPERLMGTDCKFVGNMSTLVQIQLGPIISCLHNHFFFLHK- | SEQ ID No. 183 |
| >SIP88_2\|PQ4\|−\|2 | MEKKVTKHKK- | SEQ ID No. 184 |
| >SIP27_0\|PQ4\|+\|l | MRKALDITRKPLSWSTGHTK- | SEQ ID No. 185 |
| >SIP27_1\|PQ4\|+\|3 | MEKAEHLSSSAHEKSS- | SEQ ID No. 186 |
| >SIP27_3\|PQ4\|−\|3 | MIQKSQTRLVIKLYFV- | SEQ ID No. 187 |
| >SIP130_0\|PQ4\|+\|1 | MNPTIDPTTI- | SEQ ID No. 188 |
| >SIP130_1\|PQ4\|−\|1 | MDFGSYGFNLPVLVMSRSYGGGGG- | SEQ ID No. 189 |
| >SIP52_0\|PQ414-11 | MHSQYYSPNHGY- | SEQ ID No. 190 |
| >SIP52_1\|PQ4\|+\|2 | MATNKIRLGTKNQTE- | SEQ ID No. 191 |
| >SIP52_3\|PQ4\|−\|1 | MKAFHYYQPKEIRPLLDIRFMPERYEVGLIWKHWAIYFIP | SEQ ID No. 192 |
| | TILSGFWFLVLFY- | |
| >SIP52_4\|PQ4\|−\|1 | MYSDEMLLILGGL- | SEQ ID No. 193 |
| >SIP174_0\|PQ4\|+\|1 | MADLSPNLLKQPKGLALSRF- | SEQ ID No. 194 |
| >SIP174_2\|PQ4\|+\|2 | MVLASSLLGARPFVG- | SEQ ID No. 195 |
| >SIP174_3\|PQ4\|−\|1 | MSRNLTHRPKRRENTRT- | SEQ ID No. 196 |
| >SIP174_4\|PQ4\|−\|1 | MDELQGGRRQAPCFQRSGGPNLI- | SEQ ID No. 197 |
| >SIP174_5\|PQ4\|−\|3 | MLSEKWRSESYLNCENN- | SEQ ID No. 198 |
| >SIP47_0\|PQ4\|+\|3 | MRNQSHVKTRHFAERFFNDSKIFFSFPDKISCFYEYPFL | SEQ ID No. 199 |
| | DSFRKLLFFVDLEICIDDLKSKVESLSLLLILFQFVLQLI- | |
| >SIP47_1\|PQ4\|−\|1 | MGTRKNTRFYQGRRRKFWSR- | SEQ ID No. 200 |
| >SIP60_1\|PQ4\|+\|3 | MEYKCFNSDFTAVEK- | _SEQ ID No. 201 |
| >SIP60_2\|PQ4\|−\|1 | MLMVKMDQKDPDTISMSLFSCFLTFPLQ- | SEQ ID No. 202 |
| >SIP168_0\|PQ4\|+\|2 | MAYDVRGNNVTTHVMGPFSGPSEVWLGP- | SEQ ID No. 203 |
| >SIP168_1\|PQ4\|+\|3 | MLWAHLVAQVKSGLALKSMEGLM- | SEQ ID No. 204 |
| >SIP146_0\|PQ4\|+\|1 | MPPPPKRQAVGPRVYAIAGEEDVDEDGADPIVGKSS- | SEQ ID No. 205 |
| >SIP146_1\|PQ4\|+\|2 | MLMRMAPTRLLVSLLKNLFK- | SEQ ID No. 206 |
| >SIP146_2\|PQ4\|+\|3 | MSIACACFWCSCLS- | SEQ ID No. 207 |
| >SIP146_3\|PQ4\|−\|2 | MAYTRGPTACRFGGGGIIPLF- | SEQ ID No. 208 |
| >SIP64_0\|PQ4\|+\|2 | MMPRRPTRRLYSSLKTPLRHDA- | SEQ ID No. 209 |
| >SIP64_1\|PQ4\|−\|1 | MRRLGSLDLLATGFFSDLEIRWCIALVLFFLLFVKPPRFFI | SEQ ID No. 210 |
| | VFARSWSLWCLULLFCT- | |
| >SIP64_3\|PQ4\|−\|3 | MFLLLSCSIVLFQISIAHRL- | SEQ ID No. 211 |
| >SIP46_0\|PQ4\|+\|2 | MNVRIHPITN- | SEQ ID No. 212 |
| >SIP46_2\|PQ4\|+\|3 | MSGYIRSQTNTRAHEY- | SEQ ID No. 213 |
| >SIP117_0\|PQ4\|+\|3 | MFFLQLQLHRGLV- | SEQ ID No. 214 |
| >SIP117_1\|PQ4\|+\|3 | MKRTLKAQRMILKLLPVSKMHCLSNL- | SEQ ID No. 215 |
| >SIP158_1\|PQ4\|+\|3 | MSLVYWVALFHVHAVNNFHLPQP- | SEQ ID No. 216 |
| >SIP158_3\|PQ4\|−\|1 | MHMEEGHPIDQAHQIYQARQNDVQNPSALATTIYNRHF | SEQ ID No. 217 |
| | FGFSIVQLKLVPNCNMLSIGSMVFFL- | |
| >SIP158_4\|PQ4\|−\|2 | MFFTLTIESAVCFINRRRGLGSKGDHRDQG- | SEQ ID No. 218 |
| >SIP30_0\|PQ4\|+\|3 | MPCCLAPSPDWTRH- | SEQ ID No. 219 |
| >SIP30_2\|PQ4\|−\|2 | MSGPVRRWSEATRHVRRDRSGNALFM- | SEQ ID No. 220 |
| >SIP33_0\|PQ4\|+\|3 | MEASGTKVPSESRNIALLLDRL- | SEQ ID No. 221 |
| >SIP23_0\|PQ4\|+\|3 | MSNPELISHTQQKELRWQRLHLVM- | SEQ ID No. 222 |
| >SIP23_2\|PQ4\|−\|1 | MIRCFCVTTKVLSFFRFKCCITLV- | SEQ ID No. 223 |
| >SIP23_3\|PQ4\|−\|3 | MFLCDYQSFELF- | SEQ ID No. 224 |
| >SIP23_4\|PQ4\|−\|3 | MLYHFGVGSWWFIR- | SEQ ID No. 225 |
| >SIP23_5\|PQ4\|−\|3 | MTRCSLCHRSSFC- | SEQ ID No. 226 |
| >SIP56_1\|PQ4\|−\|2 | MNPESSSAES- | SEQ ID No. 227 |
| >SIP56_3\|PQ4\|−\|3 | MRMRRGTRRRASR- | SEQ ID No. 228 |
| >SIP132_0\|PQ4\|+\|2 | MISDPSFAEALHKRRLSSAKRRW- | SEQ ID No. 229 |
| >SIP132_2\|PQ4\|−\|3 | MIVSSCAKPQRMRDQRSCVA- | SEQ ID No. 230 |
| >SIP132_3\|PQ4\|−\|3 | MGKPRIKSSTIPSHLLFLGLR- | SEQ ID No. 231 |
| >SIP21_0\|PQ4\|+\|1 | MLWPIPSRRHPRQTSKDPKEAKAIAKQTVMSQKLLGEG | SEQ ID No. 232 |
| | SSQF- | |
| >SIP21_1\|PQ4\|−\|1 | MPSCLDRKPNLLKL- | SEQ ID No. 233 |
| >SIP15_1\|PQ4\|+\|2 | MYYPNIKPENNLNFVF- | SEQ ID No. 234 |
| >SIP15_3FQ4\|−\|1 | MNHDLYLLKYSVLCEVNIGPVLG- | SEQ ID No. 235 |
| >SIP15_4\|PQ4\|−\|1 | MSSVLILIAFQD- | SEQ ID No. 236 |
| >SIP15_5\|PQ4\|−\|2 | MYTLVLLNVKCRRCLY- | SEQ ID No. 237 |
| >SIP15_6\|PQ4\|−\|3 | MLNWGAYINCFSRLENKVKIVFRFNVWVVHIY- | SEQ ID No. 238 |
| >SIP100_0\|PQ4\|+\|1 | MYTIMRFSYRISSTSSRFR- | SEQ ID No. 239 |
| >SIP100_1\|PQ4\|−\|3 | MILHLKVHLNLELVLEIR- | SEQ ID No. 240 |
| >SIP101_2\|PQ4\|−\|1 | MFRADELLEKDEVTSFVRRAS- | SEQ ID No. 241 |
| >SIP101_3\|PQ4\|−\|2 | MTKLCFELTSFLRRTR- | SEQ ID No. 242 |
| >SIP173_0\|PQ4\|+\|1 | MYRTLSTRSS- | SEQ ID No. 243 |
| >SIP173_2\|PQ4\|+\|2 | MIEARTLSGQA- | SEQ ID No. 244 |
| >SIP173_3\|PQ4\|−\|2 | MMTWSREYDTSV- | SEQ ID No. 245 |
| >SIP42_0\|PQ4\|+\|1 | MLPPSYFPLTRGVYLPRFLLDHLNTSVIVTHGRRCETHLI | SEQ ID No. 246 |
| | LEEGQVCMPPPLLV- | |
| >SIP42_1\|PQ4\|+\|2 | MISPLSELIASSFSLVRDSTFFSRTKS- | SEQ ID No. 247 |
| >SIP42_2\|PQ4\|+\|3 | MIRTGHASTVIFSFNPGGISSTLSS- | SEQ ID No. 248 |
| >SIP42_3\|PQ4\|−\|1 | MTYKGAGRFTSRKKLSPRYVGPYKVIKRVGAVAYKLDL | SEQ ID No. 249 |
| | PPKLNAFHNVFHVSQLRKCLSDQEESVEDIPPGLKENM | |
| | TVEAWPVRIMDRMTKGTREKSRDLLKVLWNCGGREEY | |
| | TWETENKMKANFPEWFKEMGKDQLDADSRTNPIQGGE | |
| | TCNARDPQ- | |
| >SIP42_4\|PQ4\|−\|2 | MRFTTSSMCHNYGSV- | SEQ ID No. 250 |
| >SIP42_5\|PQ4\|−\|2 | MRIRGRIQFKGGRLVTPAILNRIVG- | SEQ ID No. 251 |
| >SIP42_6\|PQ4\|−\|3 | MAGTDHGPNDERDSGKIKGFVKSLVELWRP- | SEQ ID No. 252 |
| >SIP131_2\|PQ4\|+\|2 | MILFCLRLLKLSTAHSRLKRSASKRDAIFEYDRRLWNRGLPE- | SEQ ID No. 253 |
| >SIP131_3\|PQ4\|+\|2 | MLHLPKLSIGLLQV- | SEQ ID No. 254 |
| >SIP131_4\|PQ4\|+\|3 | MQFSNMIDDYGTEVYLNDHEESRRPHSSSLV- | SEQ ID No. 255 |
| >SIP131_5\|PQ4\|−\|2 | MQRRGWQQTNQMHLQQIHPSPHLLIPIILGEARCLVLVS | SEQ ID No. 256 |
| | VTLDALGLNKR- | |
| >SIP140_0\|PQ4\|+\|1 | MTGISCDIWMTSFLQRTIG- | SEQ ID No. 257 |
| >SIP140_3\|PQ4\|−\|1 | MTSWVLIEVLISLRSNQPIVRCRNDVIQISQEIPVMIWPSF | SEQ ID No. 258 |
| | LTRLTSFSLIRSRAKPMTRL- | |
| >SIP140_4\|PQ4\|−\|2 | MAMFNISEAHHR- | SEQ ID No. 259 |
| >SIP140_5\|PQ4\|−\|2 | MSSRYRRRSRS- | SEQ ID No. 260 |
| >SIP61_3\|PQ4\|+\|3 | MQPFVGTRSTLHVLAETPLHP- | SEQ ID No. 261 |
| >SIP61_4\|PQ4\|−\|1 | MQGMDVTFRKGERNGCDLSEGCKERM- | SEQ ID No. 262 |
| >SIP61_5\|PQ4\|−\|3 | MQKTDVTFWKDARNGCDLSEG- | SEQ ID No. 263 |
| >SIP61_6\|PQ4\|−\|3 | MLWEASIRPLYKGGRPRPLE- | SEQ ID No. 264 |
| >SIP1_1\|PQ4\|+\|3 | MTGIKRRRRLLF- | SEQ ID No. 265 |
| >SIP1_2\|PQ4\|−\|2 | MFIFDGLKFKKI- | SEQ ID No. 266 |
| >SIP116_0\|PQ4\|+\|2 | MKFMDLVDPILVHGFSAEI- | SEQ ID No. 267 |
| >SIP116_1\|PQ4\|+\|3 | MEALRPKSCSSTPSTFH- | SEQ ID No. 268 |
| >SIP159_0\|PQ4\|−\|1 | MHELILLYTKQKRPKTIKAPGIPAPTAIA- | SEQ ID No. 269 |
| >SIP159_1\|PQ4\|−\|3 | MGLSWDLWGWDETTPFNA- | SEQ ID No. 270 |
| >SIP135_0\|PQ4\|+\|2 | MATTRLLTCARERRRILRPSKRDQRKNKRDT- | SEQ ID No. 271 |
| >SIP171_0\|PQ4\|+\|1 | MVISFAGYSPKESAR- | SEQ ID No. 272 |
| >SIP171_1\|PQ4\|+\|2 | MISPRVLGIHL- | SEQ ID No. 273 |
| >SIP171_2\|PQ4\|+\|2 | MDRSKPLRIAYYGK- | SEQ ID No. 274 |
| >SIP171_3\|PQ4\|+\|3 | MTFLIVVTDRSP- | SEQ ID No. 275 |
| >SIP171_5\|PQ4\|−\|3 | MIVTRKLGIGSGSSYLP- | SEQ ID No. 276 |
| >SIP70_1\|PQ4\|+\|2 | MTAQRMSTIGMKWIAFSADLNRDLFFFPG- | SEQ ID No. 277 |
| >SIP10_0\|PQ4\|−\|1 | MLNWLTEKC- | SEQ ID No. 278 |
| >SIP10_1\|PQ4\|−\|1 | MLNCVLAGKC- | SEQ ID No. 279 |
| >SIP10_3\|PQ4\|−\|2 | MLNCVLAENVELRFGGKMLNWLAGKC- | SEQ ID No. 280 |
| >SIP10_4\|PQ4\|−\|2 | MLIWFGGKMLNVFFGGKMLNCVLAGKC- | SEQ ID No. 281 |
| >SIP10_5\|PQ4\|−\|3 | MLNCVFDGKMLNCVLVEKC- | SEQ ID No. 282 |
| >SIP10_6\|PQ4\|−\|3 | MLNCVLAEKC- | SEQ ID No. 283 |
| >SIP10_7\|PQ4\|−\|3 | MSFWRENVECRFDGKMLNWFGRKMLNCFLAGKC- | SEQ ID No. 284 |
| >SIP10_8\|PQ4\|−\|3 | MLIWFGGKMLNVFFGGKM- | SEQ ID No. 285 |
| >SIP98_1\|PQ4\|−\|1 | MVVVSVRDSKEVHDGRVMVEAMISWLRV- | SEQ ID No. 286 |
| >SIP98_2\|PQ4\|−\|3 | MYWIIVGFLARFGDAVEC- | SEQ ID No. 287 |
| >SIP169_2\|PQ4\|+\|3 | MLMLLKHDLFL- | SEQ ID No. 288 |
| >SIP169_3\|PQ4\|−\|1 | MQKLLHGGLFGILS- | SEQ ID No. 289 |
| >SIP9_1\|PQ4\|−\|2 | MIFAMTKFQNGKKKKKQIPKVVVELMGCDA- | SEQ ID No. 290 |
| >SIP9_3\|PQ4\|−\|3 | MEKKKKNKFQNGWN- | SEQ ID No. 291 |
| >SIP51_0\|PQ4\|−\|1 | MRASKSTKKVFYSIIELSFKGGQETLCCVRMSR- | SEQ ID No. 292 |
| >SIP51_2\|PQ4\|−\|2 | MLSCERVSRRRKSSILLSSCRSKAVKKPFVAFECPVEG | SEQ ID No. 293 |
| | STISYCCC- | |
| >SIP151_0\|PQ4\|−\|2 | MQFPVITQCY- | SEQ ID No. 294 |
| >SIP75_4\|PQ4\|+\|3 | MSTTKQSGVGRLMLLRFFSLNCFGCYVVGILIDLCH- | SEQ ID No. 295 |
| >SIP75_5\|PQ4\|−\|1 | MHRKKSIFHYQYCVKIMRLLILLQIYNYRSINGRDLSKSQ | SEQ ID No. 296 |
| | QHNIQNS- | |
| >SIP14_4\|PQ4\|−\|1 | MSYGLTQLILLNGLDQPITHLI- | SEQ ID No. 297 |
| >SIP14_5\|PQ4\|−\|3 | MSLKLNELVVVDPTHFVKVVVGSTHNSFNLMG- | SEQ ID No. 298 |
| >SIP50_0\|PQ4\|+\|1 | MVDTGPVRSVLYS- | SEQ ID No. 299 |
| >SIP50_1\|PQ4\|+\|2 | MLRFLWWILVLLGRYCTVDGAAT- | SEQ ID No. 300 |
| >SIP50_4\|PQ4\|+\|3 | MGLLPKCWTFTDWYQPLMGEIMLLLGLTFSWPIVQEKVI | SEQ ID No. 301 |
| | TSSLRHRYRN- | |
| >SIP50_5\|PQ4\|−\|2 | MSDPIACLECQHET- | SEQ ID No. 302 |
| >SIP50_6\|PQ4\|−\|3 | MKPNYDSGGEDLK- | SEQ ID No. 303 |
| >SIP20_1\|PQ4\|+\|3 | MAFKSRKIGKNKSNIKENKRKGKEKSNQWVASH- | SEQ ID No. 304 |
| >SIP20_2\|PQ4\|−\|1 | MPLGTMSFKSRGGSY- | SEQ ID No. 305 |
| >SIP62_0\|PQ4\|+\|2 | MNAGRDRYSVPLHRKSNNPS- | SEQ ID No. 306 |
| >SIP119_0\|PQ4\|−\|2 | MYLSLMILQVMQSY- | SEQ ID No. 307 |
| >SIP77_2\|PQ4\|−\|1 | MHEPVAVDGPLGLKPGLAL- | SEQ ID No. 308 |
| >SIP77_3\|PQ4\|−\|2 | MSLWRLMDHWASSLALLCRLCSIEPERPLMCI- | SEQ ID No. 309 |
| >SIP37_0\|PQ4\|+\|1 | MRLGESEVFNSAQQSAEIRLFRRMAFGSQNYSTDFFSH | SEQ ID No. 310 |
| | SSYNSRDANV- | |
| >SIP75_6\|PQ4\|−\|2 | MAEIYQNPNNITSKTVKTKET- | SEQ ID No. 311 |
| >SIP37_2\|PQ4\|−\|2 | MIFTRLRLCYCNYYARKNLYCNFGYQMPFF- | SEQ ID No. 312 |
| >SIP37_3\|PQ4\|−\|3 | MSTNQEKAKPEGDSYCFNHSEKAQL- | SEQ ID No. 313 |
| >SIP37_4\|PQ4\|−\|3 | MVCATNGERRKRTICV- | SEQ ID No. 314 |
| >SIP37_5\|PQ4\|−\|3 | MREKICTVILATKCHSSKQSDLC- | SEQ ID No. 315 |
| >SIP139_0\|PQ4\|+\|2 | MATPFFPIDPLSFSQLTHPRETDFAES- | SEQ ID No. 316 |
| >SIP139_2\|PQ4\|+\|3 | MMISMILPWLLLFFQ- | SEQ ID No. 317 |
| >SIP143_0\|PQ4\|+\|1 | MYKMYACMDVCMNACMDVWMYA- | SEQ ID No. 318 |
| >SIP143_1\|PQ4\|+\|1 | MHRYFVLGCRFSGFVKLDVEEADQVLIVLCGLT- | SEQ ID No. 319 |
| >SIP143_2\|PQ4\|+\|1 | MLLYLVYQVGTGTYQ- | SEQ ID No. 320 |
| >SIP143_4\|PQ4\|+\|2 | MHVWMYGCMHECMYGCMHECMYGCMHECMYGCMH | SEQ ID No. 321 |
| | ECTDILFWDADFPGS- | |
| >SIP143_5\|PQ4\|+\|2 | MWKRLIKYSSCFVG- | SEQ ID No. 322 |
| >SIP143_7\|PQ4\|+\|3 | MYGCMHECMYGCMDVCMNACMDVCMNACMDVCMN | SEQ ID No. 323 |
| | ACMDVCMNAPIFCFGMQIFRVRETGCGRG- | |
| >SIP41_0\|PQ4\|+\|1 | MASSCQLLAINVGEGFAAVVAASFGRSRANSFNSEVVP | SEQ ID No. 324 |
| | DVRLLPPALGG- | |
| >SIP122_2\|PQ4\|+\|2 | MRSVRFLDSILSYNNFVLSN- | SEQ ID No. 325 |
| >SIP122_3\|PQ4\|−\|3 | MFGLRLLRRNLLQCF- | SEQ ID No. 326 |
| >SIP150_1\|PQ4\|−\|1 | MQVRGELFNRCLV- | SEQ ID No. 327 |
| >SIP150_2\|PQ4\|−\|2 | MQIAKEYTCTPSHSFSLFICCMLRSKSFLRCKLEGNSSID | SEQ ID No. 328 |
| | VLSRKSTEILFEISGNILKREHPLRSRR- | |
| >SIP150_4\|PQ4\|−\|3 | MSCLEKALRYCLRYQETSSNVSIL- | SEQ ID No. 329 |
| >SIP141_0\|PQ4\|+\|1 | MLKDISMRQETRPLTKQNFT- | SEQ ID No. 330 |
| >SIP141_1\|PQ4\|+\|3 | MFMFEFCSLWMCFEFECIVFE- | SEQ ID No. 331 |
| >SIP141_2\|PQ4\|+\|3 | MQITYRDDTINMRIE- | SEQ ID No. 332 |
| >SIP141_3\|PQ4\|−\|1 | MSGQISLTLFSCLLCHLDRLFAFQEPSLGLFGLLTLSKNT | SEQ ID No. 333 |
| | PYLETDTFTDTKITQTQCTQTQNTSKDYKTQT- | |
| >SIP141_4\|PQ4\|−\|2 | MLISLSICQVRYLSLYSHVYCVISIGYLHFKNRVLACLGY | SEQ ID No. 334 |
| | SHYPKTHLI- | |
| >SIP39_0\|PQ4\|+\|1 | MNIGTLLLMFYVSKR- | SEQ ID No. 335 |
| >SIP39_1\|PQ4\|+\|1 | MNRLVFLAKEVNNH- | SEQ ID No. 336 |
| >SIP39_2\|PQ4\|+\|2 | MLANAEVSIYKSLSRTL- | SEQ ID No. 337 |
| >SIP39_3\|PQ4\|+\|2 | MFMSSGFIDSTKRVSSI- | SEQ ID No. 338 |
| >SIP39_4\|PQ4\|−\|1 | MYHKDSIFLLLLWL- | SEQ ID No. 339 |
| >SIP39_6\|PQ4\|−\|2 | MKTGDAYLSSGMYIHEIEMKKFFNGYSLLWLRRLTGSY | SEQ ID No. 340 |
| | TRDTFSGVYKTRGHEHRLQCTTKILSFFFFLWFSEFLFL- | |
| >SIP39_7\|PQ4\|−\|3 | MFKLWGFLTQG- | SEQ ID No. 341 |
| >SIP39_8\|PQ4\|−\|3 | MLIFPLECTSTRLK- | SEQ ID No. 342 |
| >SIP39_9\|PQ4\|−\|3 | MNIVFNVPQRFYLSSSSCGSLSFYFCKSTQCIYRVLDNDL- | SEQ ID No. 343 |
| >SIP34_2\|PQ4\|−\|3 | MVFIESIFKDWRAEEAS- | SEQ ID No. 344 |
| >SIP90_1\|PQ4\|+\|1 | MLTQILPKHLQFKTNSLTTRIRDTP- | SEQ ID No. 345 |
| >SIP90_2\|PQ4\|−\|1 | MMVLIIRFARHSYDALILFLMIYQYRVSISCVFLCFPELLW | SEQ ID No. 346 |
| | SISNSSSQTIGFELQMFWQNLGQHFLVKICFFSQHFS- | |
| >SIP90_3\|PQ4\|−\|2 | MFFFHSLSSCDYRS- | SEQ ID No. 347 |
| >SIP90_4\|PQ4\|−\|3 | MVCFSFIHSLHVIIDHDGSNYPFCKTFIRRSYFIFNDLSV- | SEQ ID No. 348 |
| >SIP74_0\|PQ4\|+\|1 | MMTYLSFSEALVIYHPHIISS- | SEQ ID No. 349 |
| >SIP11_1\|PQ4\|+\|3 | MTKVRSISSLVMITTPKATSSIIPIRRRQLLVEI- | SEQ ID No. 350 |
| >SIP11_2\|PQ4\|+\|3 | MKKENGIGTQMKKIITSFHILKKMSRSQQERSHQVKSLL | SEQ ID No. 351 |
| | HDQLH- | |
| >SIP11_4\|PQ4\|−\|2 | MWKEVIIFFI- | SEQ ID No. 352 |
| >SIP11_5\|PQ4\|−\|2 | MSMSNTSKDS- | SEQ ID No. 353 |
| >SIP11_6\|PQ4\|−\|2 | MRNTGLSSAPSFLWCFSGDTFCWRSI- | SEQ ID No. 354 |
| >SIP11_7\|PQ4\|−\|3 | MAFLQVFFSMASWKSMGSHSANKQNKVRFS- | SEQ ID No. 355 |
| >SIP11_8\|PQ4\|−\|3 | MKIYFSLLSSSLLRFSSGT- | SEQ ID No. 356 |
| >SIP11_9\|PQ4\|−\|3 | MLLAISRIVLFFLSATPFC- | SEQ ID No. 357 |
| >SIP57_2\|PQ4\|−\|2 | MLRRMILQRNR- | SEQ ID No. 358 |
| >SIP128_1\|PQ4\|+\|2 | MSRANISQSI- | SEQ ID No. 359 |
| >SIP128_3\|PQ4\|−\|1 | MFARLMIKVS- | SEQ ID No. 360 |
| >SIP128_4\|PQ4\|−\|2 | MSLSILRKLGPHR- | SEQ ID No. 361 |
| >SIP66_0\|PQ4\|+\|1 | MNHSNAPRICSPNLITISRKKAIIFFI- | SEQ ID No. 362 |
| >SIP66_1\|PQ4\|+\|2 | MKNCWKLPSTGLP- | SEQ ID No. 363 |
| >SIP66_2\|PQ4\|+\|3 | MSLHICSSKQSCPIQ- | SEQ ID No. 364 |
| >SIP66_3\|PQ4\|+\|3 | MTREQQPLYVLFPSMLACMLV- | SEQ ID No. 365 |
| >SIP66_4\|PQ4\|−\|2 | MKKIIAFFLDIVIKLGEQILGALLWFMEAQLMGASNNSSF | SEQ ID No. 366 |
| | LFKHQSVVF- | |
| >SIP66_5\|PQ4\|−\|2 | MLVDLLLDWTGLF- | SEQ ID No. 367 |
| >SIP66_6\|PQ4\|−\|3 | MEGKRTYNGCCSRVI- | SEQ ID No. 368 |
| >SIP16_0\|PQ4\|+\|3 | MICHVIKLEKSVEGGKVTRAWVARKVLTPSRNGGKWD | SEQ ID No. 369 |
| | SPLISAVGKVEKS- | |
| >SIP167_0\|PQ4\|+\|1 | MVLKLGLLCTNLVPESRPDMVKWQYLDRQVSLPDFSP | SEQ ID No. 370 |
| | DSPGIGIVTPVLVGGSSTVISNISSPVTEFITHSIQYGIGR- | |
| >SIP167_1\|PQ4\|+\|3 | MNWNKKLIWPSWSKAPD- | SEQ ID No. 371 |
| >SIP167_2\|PQ4\|−\|3 | MLTVGFEPTPFRTRTLIWRLGPTRPYQLFVNNVHLQAS | SEQ ID No. 372 |
| | NFTSHRPIPY- | |
| >SIP167_3\|PQ4\|−\|3 | MLEITVELPPTKTGVTIPIPGESGEKSGNETCLSRYCTTF | SEQ ID No. 373 |
| | TMSGLDSGTRFVQSNPNFNTISI- | |
| >SIP85_0\|PQ4\|+\|1 | MALRIPKLYLVLPV- | SEQ ID No. 374 |
| >SIP85_5\|PQ4\|−\|2 | MRRIPEPVQITQGMQI- | SEQ ID No. 375 |
| >SIP156_1\|PQ4\|+\|2 | MDCFNANTNQRREATERNRTCKVTSGLSASQ- | SEQ ID No. 376 |
| >SIP156_2\|PQ4\|−\|1 | MCFRNPAVEI- | SEQ ID No. 377 |
| >SIP156_4\|PQ4\|−\|2 | MGNSCASETQL- | SEQ ID No. 378 |
| >SIP121_0\|PQ4\|−\|1 | MTVTKAVTLKPRRFMVNLKMVKPFHLWMQWQGVRWT | SEQ ID No. 379 |
| | KKLGSVLKVLSKVHTLLKAVLALKGQRMQ- | |
| >SIP121_1\|PQ4\|−\|1 | MLMVLDKFCSSSLRN- | SEQ ID No. 380 |
| >SIP125_1\|PQ4\|+\|2 | MSLGHSQRQSIHILLHLYCLHWLRYPRLD- | SEQ ID No. 381 |
| >SIP125_3\|PQ4\|−\|1 | MQQDVYALSLRVS- | SEQ ID No. 382 |
| >SIP125_4\|PQ4\|−\|2 | MMNSNFPFICFRPSDFHGRHCIQSVNYIKRLDQ- | SEQ ID No. 383 |
| >SIP175_1\|PQ4\|+\|1 | MYCPITIDQIHQTRNSIPHCC- | SEQ ID No. 384 |
| >SIP175_2\|PQ4\|+\|2 | MRRQIFSFLGLTTLSSNLLVCIAP- | SEQ ID No. 385 |
| >SIP175_3\|PQ4\|−\|3 | MGQYIPKGSKKESLSRES- | SEQ ID No. 386 |
| >SIP25_2\|PQ4\|−\|3 | MKIHDQNSPISTIDSVNEQLPFLITDSNPFAQ- | SEQ ID No. 387 |
| >SIP25_3\|PQ4\|−\|1 | MGDFNSAAVMIH- | SEQ ID No. 388 |
| >SIP25_4\|PQ4\|−\|2 | MVHGFTPHTRITGQKDLSQ- | SEQ ID No. 389 |
| >SIP25_5\|PQ4\|−\|3 | MDLLLILELLGKRI- | SEQ ID No. 390 |
| >SIP25_6\|PQ4\|−\|3 | MNFHGRFQLGGGDDSLTSFQS- | SEQ ID No. 391 |
| >SIP69_0\|PQ4\|−\|1 | MKKTLIIMPGKNQNQRKKEQREI- | SEQ ID No. 392 |
| >SIP69_1\|PQ4\|−\|2 | MIDSSPFSNYAIFNNV- | SEQ ID No. 393 |
| >SIP137_0\|PQ4\|+\|1 | MPHQTQREEQDLLYL- | SEQ ID No. 394 |
| >SIP137_3\|PQ4\|+\|2 | MRTMQRCHIKLREKNRISYTYRQGKYNRCSIYLFQKDTY | SEQ ID No. 395 |
| | SRNSCFQ- | |
| >SIP137_4\|PQ4\|−\|1 | MLHLLYFPCL- | SEQ ID No. 396 |
| >SIP137_5\|PQ4\|−\|1 | MWHLCMVLISLRSIWNYVLSFLVKAINTSF- | SEQ ID No. 397 |
| >SIP137_6\|PQ4\|−\|2 | MSFLNRYLSEKGRCCICYIFPVYKYKRSCSSL- | SEQ ID No. 398 |
| >SIP118_0\|PQ4\|+\|1 | MLKRVGNNSKHASGNNISLIGETLPTTSLASFSCLLLVCS | SEQ ID No. 399 |
| | VAFFSGKKLRKCSCLPPSFLNRL- | |
| >SIP118_3\|PQ4\|−\|1 | MLFPEACFELFPTRLSIT- | SEQ ID No. 400 |
| >SIP118_4\|PQ4\|−\|2 | MGEDTNISLAFCQKRMPQSIQVKDMKMMRVRL- | SEQ ID No. 401 |
| >SIP35_1\|PQ4\|−\|2 | MKKARLQSPLCSRVNNKIAAYQSHTV- | SEQ ID No. 402 |
| >SIP80_1\|PQ41\|−\|2 | MAYFRSASRNSPTFLNR- | SEQ ID No. 403 |
| >SIP80_3\|PQ4\|−\|3 | MHRLLLIHPN- | SEQ ID No. 404 |
| >SIP80_4\|PQ4\|−\|3 | MLGSFVKRYGNTP- | SEQ ID No. 405 |
| >SIP110_0\|PQ4\|+\|1 | MNKRRPPTYNKMERLMESKYMMIWILKMKMEVFKR- | SEQ ID No. 406 |
| >SIP110_2\|PQ4\|−\|1 | MVIGYLLNTSIFILSIHIIMYLDSINLSILL- | SEQ ID No. 407 |
| >SIP28_0\|PQ4\|+\|1 | MKKRRRRKKISSNKSSICKLQKKKTAKVTKEEVVVKYED | SEQ ID No. 408 |
| | EVVVDMEMDVVGGHMKTTQTKEVKTHQEVVGKDTQN | |
| | QDTINQVSNATIGSLDIMLLNVKLLATKNLRRRPTTLKK | |
| | KFKKKTCY- | |
| >SIP28_1\|PQ4\|+\|1 | MNKKRIISGTSIWQVITCAGEKVCSRSLMNR- | SEQ ID No. 409 |
| >SIP28_2\|PQ4\|+\|1 | MEIINLFPTFTIFRA- | SEQ ID No. 410 |
| >SIP28_3\|PQ4\|+\|1 | MTLHNVLRCVTKRSLGYGIFDSDI- | SEQ ID No. 411 |
| >SIP28_4\|PQ4\|+\|1 | MCWQSSRNHLVKVITSFSLLMIFQEKHGYIF- | SEQ ID No. 412 |
| >SIP28_5\|PQ4\|+\|1 | MLMYRMRSGAN- | SEQ ID No. 413 |
| >SIP28_6\|PQ4\|+\|1 | MTKVRSISSLVMITTPKATSSIIPIRRRQLLVEI- | SEQ ID No. 414 |
| >SIP28_7\|PQ4\|+\|1 | MKKENGIGTQMKKIITSFHILKKMSRSQQERSHQVKSLL | SEQ ID No. 415 |
| | HHQLHQQVLK- | |
| >SIP28_8\|PQ4\|+\|1 | MPWMKRSNQYKRMTHGS- | SEQ ID No. 416 |
| >SIP28_9\|PQ4\|+\|2 | MLQLWEVWTLCF- | SEQ ID No. 417 |
| >SIP28_10\|PQ4\|+\|2 | MASSIRTSKFWRIGVAFKEGNGERATLYKSSKSSV- | SEQ ID No. 418 |
| >SIP28_11\|PQ4\|+\|2 | MSTWKAIQNELSKGVKFKSTKIVGANTYRCVVVSNQAEITVV- | SEQ ID No. 419 |
| >SIP28_12\|PQ4\|+\|2 | MGIFFERKIRGVRNFQKV- | SEQ ID No. 420 |
| >SIP28_13\|PQ4\|+\|2 | MGVNFTSKWTQDNWREVGV- | SEQ ID No. 421 |
| >SIP28_14\|PQ4\|−\|1 | MSFHLFGLCCLHVASNHVHFHIHHDLVLVLDHHLFFGNF | SEQ ID No. 422 |
| | GRFLLL- | |
| >SIP28_16\|PQ4\|−\|2 | MWKEVIIFFI- | SEQ ID No. 423 |
| >SIP28_17\|PQ4\|−\|2 | MSMSNTSKDS- | SEQ ID No. 424 |
| >SIP28_18\|PQ4\|−\|2 | MRNTGLSSAPSFLWCFSGDTFCWRSI- | SEQ ID No. 425 |
| >SIP28_19\|PQ4\|−\|2 | MGFKLFENFEHLGFFFQKIYPCFS- | SEQ ID No. 426 |
| >SIP28_20\|PQ4\|−\|2 | MISILQSNENVSFTFYLHLRFIS- | SEQ ID No. 427 |
| >SIP28_21\|PQ4\|−\|2 | MILFLFIFLVASH- | SEQ ID No. 428 |
| >SIP28_22\|PQ4\|−\|2 | MSKLPTIVAFDT- | SEQ ID No. 429 |
| >SIP28_23\|PQ4\|−\|2 | MWPPTTSISISTTTSSSYLTTTSSLVTLAVFFFCNLHIEDL | SEQ ID No. 430 |
| | FDDIFFULLFFISL- | |
| >SIP28_24\|PQ4\|−\|3 | MAFLQVFFSMASWKSMGSHSANKQNKVRFS- | SEQ ID No. 431 |
| >SIP28_25\|PQ4\|−\|3 | MKIYFSLLSSSLLRFSSGT- | SEQ ID No. 432 |
| >SIP28_26\|PQ4\|−\|3 | MLLAISRIVLFFLSATPFC- | SEQ ID No. 433 |
| >SIP28_27\|PQ4\|−\|3 | MPLSSQYLRNSLDVNSPPRSDLMVLITRPLSFSTWALNF | SEQ ID No. 434 |
| | LKISNTSDFSFKKYTHVFLEKSSIKRRK- | |
| >SIP28_28\|PQ4\|−\|3 | MSFRMLRTNILFFDIGTLVMRLLSWSLIERLLSFNLIS- | SEQ ID No. 435 |
| >SIP28_29\|PQ4\|−\|3 | MRMFPLPFTSIFDSSPKATFPLTDSSSSANILFLPHM- | SEQ ID No. 436 |
| >SIP28_30\|PQ4\|−\|3 | MISSFFFFFSS- | SEQ ID No. 437 |
| >SIP49_0\|PQ4\|−\|2 | MSPFSFSRTTGAPSVQRIFNFPFRIKRRIKKGWSVKSPV | SEQ ID No. 438 |
| | PSRTKFRINKGS- | |
| >SIP164_1\|PQ4\|−\|3 | MRTFLGTYDKCLAVSENDFSDNPTVCGAGCNG- | SEQ ID No. 439 |
| >SIP136_0\|PQ4\|−\|2 | MIKQIVISSWHKMSRHCDPFLHHTR- | SEQ ID No. 440 |
| >SIP12_2\|PQ41\|−\|2 | MTERVQWLCFTLETRLSHGCRRNNQLSLYPLVKRSM- | SEQ ID No. 441 |
| >SIP12_3\|PQ4\|+\|2 | MIEVNTLTHAITTLESVLAKRTCNWSM- | SEQ ID No. 442 |
| >SIP12_4\|PQ4\|+\|3 | MDVEETTNCHSIHL- | SEQ ID No. 443 |
| >SIP12_5\|PQ4\|+\|3 | MCMPCYLVKKPLEGVKLTTRGTNEDLCGQQVGNSFGE | SEQ ID No. 444 |
| | EPGLP- | |
| >SIP12_6\|PQ4\|−\|2 | MCLHILQLHVLFANTLSNWIACVNVFTSIMEDRVLRQSY | SEQ ID No. 445 |
| | CRLVVHKDLRWFLLW- | |
| >SIP12_8\|PQ4\|−\|3 | MCLLRSWKTGFFAKAIADLLSTKIFVGSSCGKLNSFKRF | SEQ ID No. 446 |
| | LNQIAWHTHDVAATYSASQVDRVTIGCFFDIHVKAVSPM | |
| >SIP38_1\|PQ4\|+\|2 | MLDSECLLEIYSLCLSLLLLKELNLIVWRFMRKRSIRFSK | SEQ ID No. 447 |
| | RWISYLGRLVLVLMCGVVRVILMNSCVWQRIILMKDGN- | |
| >SIP38_3\|PQ4\|−\|1 | MRWINHKEVQNSLFQFPSFINIMRCQTQEFIRITRTTPHI | SEQ ID No. 448 |
| | NTNTNLPR- | |
| >SIP38_4\|PQ4\|−\|1 | MLLFRINLHTIRFNSFKSNKLKQRL- | SEQ ID No. 449 |
| >SIP55_1\|PQ4\|−\|1 | MNLIVVYAAPSMSRRSGLWGELKEVVSGLVGLLIIGGDF | SEQ ID No. 450 |
| | ITILRVDERMGGNGRLSPDFLAFGDWIN- | |
| >SIP55_2\|PQ4\|−\|2 | MREWVEMGGFHQIF- | SEQ ID No. 451 |
| >SIP55_3\|PQ4\|−\|3 | MGRVEGSCIWFGRAVDNWRRLYHHLESG- | SEQ ID No. 452 |
| >SIP73_1\|PQ4\|+\|2 | MLLAFFTWETSMICFVDQILLPHSLYDQRK- | SEQ ID No. 453 |
| >SIP73_2\|PQ4\|−\|3 | MIKESEILYP- | SEQ ID No. 454 |
| >SIP73_3\|PQ4\|−\|2 | MDNRMRLIMGTKFHFL- | SEQ ID No. 455 |
| >SIP73_4\|PQ4\|−\|3 | MNITGTEWITECD- | SEQ ID No. 456 |
| >SIP73_5\|PQ4\|−\|3 | MSEAKEFGQRNKS- | SEQ ID No. 457 |
| >SIP105_0\|PQ4\|−\|1 | MRALTIRLETNFVSSLSFLARFSSIWMRRDWTCVWTRT | SEQ ID No. 458 |
| | KSCIYFIAFIV- | |
| >SIP13_0\|PQ4\|+\|1 | MYILKYGHFS- | SEQ ID No. 459 |
| >SIP13_1\|PQ4\|+\|1 | MTILSLDRSRRAGGSIRIVYGFAKYGS- | SEQ ID No. 460 |
| >SIP13_2\|PQ4\|+\|1 | MDIFYPIYKRYISD- | SEQ ID No. 461 |
| >SIP13_3\|PQ4\|+\|2 | MDISLDTIYLVMLKNLGYNVTIV- | SEQ ID No. 462 |
| >SIP13_6\|PQ4\|−\|2 | MNVLICFLFRCVCSFLVIYILQFS- | SEQ ID No. 463 |
| >SIP63_0\|PQ4\|+\|2 | MHGTFRFARSCLYPLQLDSTI- | SEQ ID No. 464 |
| >SIP63_1\|PQ4\|+\|3 | MGHFVLLDRACTHSNLTVLFNSRVFAFCFFSSERARGSI | SEQ ID No. 465 |
| | FNVYYVLKLYFI- | |
| >SIP154_0\|PQ4\|+\|3 | MGSGASGPVRSSQSSQAGGRFNDADPIAIDYGKY- | SEQ ID No. 466 |
| >SIP154_1\|PQ4\|−\|1 | MKKQQKTIKTSLNLEKLKSKRNLIFSVIYGDRISIIKTSTSL | SEQ ID No. 467 |
| | ATLR- | |
| >SIP154_3\|PQ4\|−\|3 | MKTNKNTKYII- | SEQ ID No. 468 |
| >SIP53_1\|PQ4\|−\|2 | MIMCGLTLRVACTRYRAVIDCYHNKRGILRKSQEWRKLL | SEQ ID No. 469 |
| | RMS- | |
| >SIP94_2\|PQ4\|+\|3 | MDVQLLHQQSYSS- | SEQ ID No. 470 |
| >SIP94_3\|PQ4\|−\|2 | MNAGRGRYTFLHTYDEYDCWWSS- | SEQ ID No. 471 |
| >SIP2_0\|PQ4\|+\|2 | MEKVFKCFERVF- | SEQ ID No. 472 |
| >SIP2_1\|PQ4\|−\|1 | MTLHNVLRCVTKRSLGYGIFDSDI- | SEQ ID No. 473 |
| >SIP2_2\|PQ4\|−\|2 | MASSIRTSKFWRIGVAFKEGNGERATLYKSSKARL- | SEQ ID No. 474 |
| >SIP2_3\|PQ4\|−\|3 | MSKNRMFVLNIRNDIAQCLKMCYKEESWLWHLRFGHLN | SEQ ID No. 475 |
| | FGGLELLSRKEMVRGLPCINHPKQGCEVVFKFKAK- | |
| >SIP95_0\|PQ4\|+\|3 | MFPSMSQCSQRATMIIGVYE- | SEQ ID No. 476 |
| >SIP95_1\|PQ41\|−\|3 | MKWFLKLKRMV- | SEQ ID No. 477 |
| >SIP95_3\|PQ4\|−\|1 | MKPFSTISHTSCAPRIAFIRRLQLS- | SEQ ID No. 478 |
| >SIP95_4\|PQ4\|−\|2 | MLLWPTYLQRPSRTYLHLILDRLDRELSCLSFLSLSNHP | SEQ ID No. 479 |
| | FEFEKDYLHSPVQ- | |
| >SIP95_5\|PQ4\|−\|3 | MEGNIVTRHL- | SEQ ID No. 480 |
| >SIP95_6\|PQ4\|−\|3 | MEKVLKCFERVF- | SEQ ID No. 481 |
| >SIP68_0\|PQ4\|+\|1 | MCKVPTKTIIIKVSFFLYKKIK- | SEQ ID No. 482 |
| >SIP68_1\|PQ4\|−\|1 | MMMVLVGTLHITYL- | SEQ ID No. 483 |
| >SIP68_2\|PQ4\|−\|1 | MPAPIIPIDLCLLLIVFGFFSFLI- | SEQ ID No. 484 |
| >SIP68_4\|PQ4\|−\|3 | MKLKTTLLYFLIQKERNFDDDGFGGHFTHYLSLVCLVGI | SEQ ID No. 485 |
| | YLYNKCAFIHLYSGRDCWLLKQILTLVTSHACTHNTY- | |
| >SIP54_0\|PQ4\|+\|1 | MISSFLIGLEKMARSLPLCTSRYRSHFFPSKRN- | SEQ ID No. 486 |
| >SIP54_2\|PQ4\|−\|2 | MRSIAGGAQRK- | SEQ ID No. 487 |
| >SIP157_0\|PQ4\|+\|3 | MNTAPDTLPQRVTV- | SEQ ID No. 488 |
| >SIP157_1\|PQ4\|+\|3 | MLSPLTLSLTQQ- | SEQ ID No. 489 |
| >SIP157_2\|PQ4\|−\|2 | MSSGSTCLLFGLPGGVSLVGGGVTETTPSTGGSSKIIIVI | SEQ ID No. 490 |
| | VVLGTGLMDSAYVKR- | |
| >SIP161_1\|PQ4\|−\|1 | MNREPIVDLLG- | SEQ ID No. 491 |
| >SIP161_2\|PQ4\|−\|2 | MTTVNRDLDR- | SEQ ID No. 492 |
| >SIP3_0\|PQ4\|−\|1 | MISILQSNKNVSFTFYLHLRFIS- | SEQ ID No. 493 |
| >SIP3_1\|PQ4\|+\|1 | MILFLFIFLVASH- | SEQ ID No. 494 |
| >SIP3_2\|PQ4\|+\|2 | MFPLPFTSIFDSSPKATFPLTDSSSSANILFLPHM- | SEQ ID No. 495 |
| >SIP3_3\|PQ4\|−\|3 | MLLNVKLLATKHLRRRPTTLKKKFKKKTCY- | SEQ ID No. 496 |
| >SIP3_4\|PQ4\|−\|3 | MNKKRIISGTSIWQVITCAGEKVCSRSLMNR- | SEQ ID No. 497 |
| >SIP107_0\|PQ4\|+\|3 | MMVHSTIQDNQRFCNIGSFRDLFSRSESKSMIVI- | SEQ ID No. 498 |
| >SIP107_1\|PQ4\|−\|1 | MNHHLQLRGEVILVEVWLQGEEDAVVVVQCPLHMFRVQ- | SEQ ID No. 499 |
| >SIP96_0\|PQ4\|+\|1 | MGDLTESPRVGSLFSFFDFCPLFFVKNV- | SEQ ID No. 500 |
| >SIP96_4\|PQ4\|−\|2 | MRSLASLSKSNRVIHS- | SEQ ID No. 501 |
| >SIP71_1\|PQ4\|−\|1 | MKEINQTKMSFSSIYTFLYWGVMPLFASSLDF- | SEQ ID No. 502 |
| >SIP71_2\|PQ4\|−\|2 | MPCSVRNVRSIFLPVF- | SEQ ID No. 503 |
| >SIP71_3\|PQ4\|−\|3 | MSDQSFFQFFKWIYQVFI- | SEQ ID No. 504 |
| >SIP18_0\|PQ4\|+\|1 | MQCMPCGHHHLS- | SEQ ID No. 505 |
| >SIP18_2\|PQ4\|+\|2 | MWKIRRLWKTKRAI- | SEQ ID No. 506 |
| >SIP18_4\|PQ4\|−\|1 | MNLVYSFGSSVLKDSAYVLEM- | SEQ ID No. 507 |
| >SIP18_5\|PQ4\|−\|2 | MMSTRHALHSSRTDLLT- | SEQ ID No. 508 |
| >SIP18_6\|PQ4\|−\|2 | MFYIALLVFQSLRIFHMSETCLLLPMVRIRPAFALEPSAIGP- | SEQ ID No. 509 |
| >SIP18_8\|PQ4\|−\|3 | MHCIQVAQIC- | SEQ ID No. 510 |
| >SIP4_1\|PQ4\|+\|1 | METISTFFTDLQ- | SEQ ID No. 511 |
| >SIP4_2\|PQ4\|+\|2 | MVTKKKFLHEIYHWYWEE- | SEQ ID No. 512 |
| >SIP4_3\|PQ4\|+\|3 | MEDKLYFDLFIFDPLAQMFVLSRFDELPFVITWIMIDLEM- | SEQ ID No. 513 |
| >SIP4_4\|PQ4\|−\|1 | MSSILNSKRPKFYCKSVKNVEIVSIFTPPNTNGKFREEIS | SEQ ID No. 514 |
| | FLSP- | |
| >SIP4_5\|PQ4\|−\|2 | MLRSFPFSLLPIPMVNFVKKFLFCHHNTSKPSPRLFLCF | SEQ ID No. 515 |
| | GHIYISKSIMIHVMTNGNSSNRDSTNICARGSNINKSKYS | |
| | LSSISKACERIP- | |
| >SIP113_0\|PQ4\|−\|1 | MVMIFSSKMCLVDHHHQSTDLLLVIHVLHLLLLLTMMLC | SEQ ID No. 516 |
| | SRNPNPNQRRLCRFMTSLCMMMRMCLSLFLSSKSLPLL | |
| | LSQLGLRMFSLLSHLLLRSTGNRTVLLSMISWETIWGRK | |
| | GQIVTERRRVVPFLMI- | |
| >SIP113_2\|PQ4\|−\|1 | MENVNHQKKIRTLVVVVH- | SEQ ID No. 517 |
| >SIP113_3\|PQ4\|−\|3 | MELPFSSRSLSAPFFPKLFPMRSSKGELFCFLCFVGED | SEQ ID No. 518 |
| | EIEEKTFSNLAD- | |
| >SIP83_0\|PQ4\|+\|1 | MCERTRTKQKKHLPTSWHDGEIFGSSFLGNNEDLDSPCI- | SEQ ID No. 519 |
| >SIP83_1\|PQ4\|+\|2 | MTERFLAAASSGITKISTVPASEEKLTIRMKSLSLEMVIRV | SEQ ID No. 520 |
| | FLFPGRKYLRVIASKPNPKFSASSDLKSVIEVF- | |
| >SIP83_2\|PQ4\|+\|2 | MSIAKASPPEYDNDPSLIVTNSPPD- | SEQ ID No. 521 |
| >SIP83_4\|PQ4\|−\|1 | MDRCRILAAMLLQLTLIRILQ- | SEQ ID No. 522 |
| >SIP83_6\|PQ4\|−\|2 | MSVRRGVCYYQRWIVVVFWRRCFCN- | SEQ ID No. 523 |
| >SIP83_7\|PQ4\|−\|3 | MTTNKVIAICQSGGEFVTIKDGSLSYSGGDAFAIDIDQNT | SEQ ID No. 524 |
| | SMTDFKSELAENFGFGLEAMTLKYFLPGNKKTLITISKDK | |
| | DFIRMVNFSSDAGTVEIFVIPEEAAAKNLSVMPASR- | |
| >SIP115_1\|PQ4\|+\|3 | MVSKDQQMLFKEVENYICLT- | SEQ ID No. 525 |
| >SIP115_2\|PQ4\|−\|1 | MRDVNRLQLWQTP- | SEQ ID No. 526 |
| >SIP115_3\|PQ4\|−\|2 | MFELMSSIYSSPPP- | SEQ ID No. 527 |
| >SIP115_4\|PQ4\|−\|2 | MLIDCNSGRLHNHSGTWPSI- | SEQ ID No. 528 |
| >SIP115_6\|PQ4\|−\|3 | MDPGICLNLCQAYIVLHLLE- | SEQ ID No. 529 |
| >SIP29_1\|PQ4\|−\|1 | MLGDDMYLDR- | SEQ ID No. 530 |
| >SIP29_2\|PQ4\|−\|2 | MTCTLIGKNLRTSHQVLMHIDRAILRQCSGTLLDL- | SEQ ID No. 531 |
| >SIP127_0\|PQ4\|+\|1 | MANLRSKAKPI- | SEQ ID No. 532 |
| >SIP127_1\|PQ4\|+\|1 | MHFTPALKLATPSLVPSSSIGFTKTATASRAIVAEISPEG | SEQ ID No. 533 |
| | TRGTTMERKVSKAETATGSSVEESTEDIAAAKKAEGDG | |
| | GRDSEVRIERIATAVATR- | |
| >SIP127_2\|PQ4\|+\|3 | MGNYLGTFINGEFKK- | SEQ ID No. 534 |
| >SIP127_3\|PQ4\|−\|1 | MKLPSKMATLISPNSLWTVTASPQPLSTVISSPPPSLCAL | SEQ ID No. 535 |
| | FSPQSLFHHLLQLSSQLLYLLSILQLKIPWPFLLCLLSFP- | |
| >SIP127_4\|PQ4\|−\|1 | MIGNLFKLVLLYFLNSPLINVPR- | SEQ ID No. 536 |
| >SIP40_0\|PQ4\|+\|1 | MNIMKPFRRCRHSSCCKL- | SEQ ID No. 537 |
| >SIP40_3\|PQ4\|+\|3 | MPPLELLQWSAI- | SEQ ID No. 538 |
| >SIP40_4\|PQ4\|−\|3 | MWLDCTYNLQQLEWRHLLKGFMMFM- | SEQ ID No. 539 |
| >SIP114_0\|PQ4\|+\|2 | MEDNILGICVSNETLF- | SEQ ID No. 540 |
| >SIP114_1\|PQ4\|−\|1 | MAPEFSMDPSRLLVTGFTS- | SEQ ID No. 541 |
| >SIP114_2\|PQ4\|−\|3 | MLSSMWPVNGFSLNLSKPNLWFNGSRVFYGSFSLTC | SEQ ID No. 542 |
| | YRFYFLII- | |
| >SIP142_0\|PQ4\|−\|2 | MLFIYKFYWHMQIYVEVKRKTKQYAIVEMSFLGNG- | SEQ ID No. 543 |
| >SIP142_1\|PQ4\|−\|3 | MKCYLYINSIGTCKYTLKSKEKQNSTQ- | SEQ ID No. 544 |
| >SIP142_2\|PQ4\|−\|3 | MASIKNIGAKRKIEVKIFCLMT- | SEQ ID No. 545 |
| >SIP82_0\|PQ4\|+\|1 | MKSECVYMMS- | SEQ ID No. 546 |
| >SIP82_2\|PQ4\|−\|1 | MVITYTFFVPL- | SEQ ID No. 547 |
| >SIP82_3\|PQ4\|−\|1 | MAPVTVIKTSCIRILISFLTEEVTSFSFFASIACRRHIQHFI | SEQ ID No. 548 |
| | SSCSSC- | |
| >SIP82_4\|PQ4\|−\|3 | MYTHSDFISN- | SEQ ID No. 549 |
| >SIP170_0\|PQ4\|+\|1 | MSLEDPLKSPNKGEKAISRPFAIARRAARLYTAWLRYRS | SEQ ID No. 550 |
| | YVVIGLKSGIPAIQAYFF- | |
| >SIP170_1\|PQ4\|−\|3 | MAGIPLLRPITT- | SEQ ID No. 551 |
| >SIP24_0\|PQ4\|+\|1 | MTSLAFSRSRIETPQVSR- | SEQ ID No. 552 |
| >SIP24_1\|PQ4\|+\|3 | MFLTSALGTLVMIFDVKC- | SEQ ID No. 553 |
| >SIP24_2\|PQ4\|−\|1 | MKQSCFKYYLMFSGLICRRGAYWLNRSKSTDVMTKLW | SEQ ID No. 554 |
| | CYTI- | |
| >SIP81_1\|PQ4\|+\|3 | MSSRNTFISV- | SEQ ID No. 555 |
| >SIP81_2\|PQ4\|−\|2 | MKVFLEDMPQPSCCKKEKQILKSAR- | SEQ ID No. 556 |
| >SIP67_0\|PQ4\|−\|1 | MYCKQQEEKKTTLREMNWRVDVKSSQAVKKLRID- | SEQ ID No. 557 |
| >SIP111_1\|PQ4\|−\|2 | MTSSIKSKMGRVTSWKKSATSTTTRVAIKDTTSSKPTIPT | SEQ ID No. 558 |
| | TPTAAPTLLTLRIRCILHSNNKVRTNLLFPTTKVSFLNNN | |
| | SREITSNHHHLGCTSAKPRSYCS- | |
| >SIP126_0\|PQ4\|+\|1 | MHDSVSRILW- | SEQ ID No. 559 |
| >SIP126_1\|PQ4\|+\|1 | MSSLCILGFQRRVNLRTFLNLRRA- | SEQ ID No. 560 |
| >SIP126_3\|PQ4\|+\|3 | MIGENQITSLDTLEHIF- | SEQ ID No. 561 |
| >SIP126_4\|PQ4\|−\|1 | MLYLKNISLSYLT- | SEQ ID No. 562 |
| >SIP126_5\|PQ4\|−\|1 | MSSVLQFHFYLPKLVREFFQNFAGLWLLSFRR- | SEQ ID No. 563 |
| >SIP126_7\|PQ4\|−\|2 | MCSRVSREVIWFSPIIYCYQGS- | SEQ ID No. 564 |
| >SIP166_0\|PQ4\|+\|2 | MECIPQNSHGVPLSLLTTQALHLTILWLVTGQT- | SEQ ID No. 565 |
| >SIP166_1\|PQ4\|+\|2 | MGSTSLLLAKGLGISTRTRILETSTLLTQ- | SEQ ID No. 566 |
| >SIP166_3\|PQ4\|−\|1 | MKMKRMAFCAKNASVLHHQLHTGVIR- | SEQ ID No. 567 |
| >SIP166_4\|PQ4\|−\|1 | MRLPRIVRLKESFCWHCE- | SEQ ID No. 568 |
| >SIP166_5\|PQ4\|−\|2 | MLVSCITNSTLVL- | SEQ ID No. 569 |
| >SIP166_6\|PQ4\|−\|2 | MGLHGNFGEYTP- | SEQ ID No. 570 |
| >SIP166_8\|PQ4\|−\|3 | MAHRLCSSLLGQQG- | SEQ ID No. 571 |
| >SIP166_9\|PQ4\|−\|3 | MGILGNTLHEIAQNSEIEGELLLAL- | SEQ ID No. 572 |
| >SIP7_2\|PQ4\|+\|3 | MEEAFSPLHLFPRICYTCLQQR- | SEQ ID No. 573 |
| >SIP7_3\|PQ4\|−\|1 | MILLSLTLLQTCIANAGK- | SEQ ID No. 574 |
| >SIP7_4\|PQ4\|−\|2 | MEHTNISFGKTRVFRGSLCDFP- | SEQ ID No. 575 |

TABLE 2
List of OSIP-encoding regions, identified by comparing TARs that were
induced by the paraquat treatment as compared to the control treatment.

| seqID | source | type | start | end | attributes |
|---|---|---|---|---|---|
| Chr1 | PQ4 | transcript | 274401 | 274540 | ID = TAR0; Note = intergenic; |
| Chr1 | PQ4 | transcript | 913399 | 913500 | ID = TAR1; Note = intergenic; |
| Chr1 | PQ4 | transcript | 3360572 | 3360749 | ID = TAR2; Note = EXON; Parent = AT1G10260.1; |
| Chr1 | PQ4 | transcript | 3361044 | 3361249 | ID = TAR3; Note = EXON; Parent = AT1G10260.1; |
| Chr1 | PQ4 | transcript | 3629412 | 3629690 | ID = TAR4; Note = intron; Parent = AT1G10890.1; |
| Chr1 | PQ4 | transcript | 4627265 | 4627398 | ID = TAR5; Note = intergenic; |
| Chr1 | PQ4 | transcript | 5375567 | 5375678 | ID = TAR6; Note = intergenic; |
| Chr1 | PQ4 | transcript | 5626708 | 5626818 | ID = TAR7; Note = EXON; Parent = AT1G16480.1; |
| Chr1 | PQ4 | transcript | 6072230 | 6072445 | ID = TAR8; Note = intergenic; |
| Chr1 | PQ4 | transcript | 6102943 | 6103109 | ID = TAR9; Note = intron; Parent = AT1G17745.1; |
| Chr1 | PQ4 | transcript | 6743224 | 6743473 | ID = TAR10; Note = intergenic; |
| Chr1 | PQ4 | transcript | 7719929 | 7720564 | ID = TAR11; Note = EXON; Parent = AT1G21945.1; |
| Chr1 | PQ4 | transcript | 7721562 | 7722022 | ID = TAR12; Note = EXON; Parent = AT1G21945.1; |
| Chr1 | PQ4 | transcript | 7905709 | 7905992 | ID = TAR13; Note = intron; Parent = AT1G22400.1; |
| Chr1 | PQ4 | transcript | 7985995 | 7986270 | ID = TAR14; Note = intergenic; |
| Chr1 | PQ4 | transcript | 8187998 | 8188142 | ID = TAR15; Note = intron; Parent = AT1G23090.1; |
| Chr1 | PQ4 | transcript | 12123039 | 12123323 | ID = TAR16; Note = EXON; Parent = AT1G33420.1; |
| Chr1 | PQ4 | transcript | 12691540 | 12691657 | ID = TAR17; Note = intergenic; |
| Chr1 | PQ4 | transcript | 13197381 | 13197628 | ID = TAR18; Note = intergenic; |
| Chr1 | PQ4 | transcript | 13842516 | 13842739 | ID = TAR19; Note = EXON; Parent = AT1G36630.1; |
| Chr1 | PQ4 | transcript | 14470563 | 14470779 | ID = TAR20; Note = EXON; Parent = AT1G38423.1; |
| Chr1 | PQ4 | transcript | 15165684 | 15165789 | ID = TAR21; Note = EXON; Parent = AT1G40113.1; |
| Chr1 | PQ4 | transcript | 15564772 | 15564883 | ID = TAR22; Note = EXON; Parent = AT1G41797.1; |
| Chr1 | PQ4 | transcript | 15949322 | 15949433 | ID = TAR23; Note = EXON; Parent = AT1G42490.1; |
| Chr1 | PQ4 | transcript | 17161729 | 17161949 | ID = TAR24; Note = intergenic; |
| Chr1 | PQ4 | transcript | 17467023 | 17467207 | ID = TAR25; Note = intergenic; |
| Chr1 | PQ4 | transcript | 17477421 | 17477530 | ID = TAR26; Note = EXON; Parent = AT1G47565.1; |
| Chr1 | PQ4 | transcript | 17931255 | 17931358 | ID = TAR27; Note = intergenic; |
| Chr1 | PQ4 | transcript | 18017908 | 18019918 | ID = TAR28; Note = EXON; Parent = AT1G48710.1; |
| Chr1 | PQ4 | transcript | 18835941 | 18836116 | ID = TAR29; Note = EXON; Parent = AT1G50810.1; |
| Chr1 | PQ4 | transcript | 21460729 | 21460870 | ID = TAR30; Note = intergenic; |
| Chr1 | PQ4 | transcript | 21748168 | 21748448 | ID = TAR31; Note = intergenic; |
| Chr1 | PQ4 | transcript | 22072961 | 22073361 | ID = TAR32; Note = intron; Parent = AT1G59950.1; |
| Chr1 | PQ4 | transcript | 22175661 | 22175839 | ID = TAR33; Note = EXON; Parent = AT1G60120.1; |
| Chr1 | PQ4 | transcript | 22366650 | 22366757 | ID = TAR34; Note = EXON; Parent = AT1G60750.1; |
| Chr1 | PQ4 | transcript | 22429087 | 22429209 | ID = TAR35; Note = intron; Parent = AT1G60900.1; |
| Chr1 | PQ4 | transcript | 24887782 | 24887916 | ID = TAR36; Note = EXON; Parent = AT1G66725.1; |
| Chr1 | PQ4 | transcript | 25795431 | 25795682 | ID = TAR37; Note = EXON; Parent = AT1G68690.1; |
| Chr1 | PQ4 | transcript | 26350557 | 26350809 | ID = TAR38; Note = EXON; Parent = AT1G69950.1; |
| Chr1 | PQ4 | transcript | 28930709 | 28931032 | ID = TAR39; Note = intergenic; |
| Chr1 | PQ4 | transcript | 28987517 | 28987743 | ID = TAR40; Note = intergenic; |
| Chr1 | PQ4 | transcript | 30108578 | 30108715 | ID = TAR41; Note = EXON; Parent = AT1G80020.1; |
| Chr2 | PQ4 | transcript | 43298 | 43812 | ID = TAR42; Note = EXON; Parent = AT2G01034.1; |
| Chr2 | PQ4 | transcript | 171093 | 171419 | ID = TAR43; Note = intergenic; |
| Chr2 | PQ4 | transcript | 1337229 | 1337369 | ID = TAR44; Note = intergenic; |
| Chr2 | PQ4 | transcript | 1353821 | 1353964 | ID = TAR45; Note = intergenic; |
| Chr2 | PQ4 | transcript | 1548828 | 1548944 | ID = TAR46; Note = intergenic; |
| Chr2 | PQ4 | transcript | 1935718 | 1936000 | ID = TAR47; Note = intergenic; |
| Chr2 | PQ4 | transcript | 3249300 | 3249527 | ID = TAR48; Note = intergenic; |
| Chr2 | PQ4 | transcript | 3298297 | 3298408 | ID = TAR49; Note = EXON; Parent = AT2G07783.1; |
| Chr2 | PQ4 | transcript | 3322914 | 3323048 | ID = TAR50; Note = intergenic; |
| Chr2 | PQ4 | transcript | 3445672 | 3445815 | ID = TAR51; Note = intergenic; |
| Chr2 | PQ4 | transcript | 3467321 | 3467542 | ID = TAR52; Note = EXON; Parent = AT2G07731.1; |
| Chr2 | PQ4 | transcript | 3481225 | 3481332 | ID = TAR53; Note = EXON; Parent = AT2G07737.1; |
| Chr2 | PQ4 | transcript | 3484867 | 3484997 | ID = TAR54; Note = intergenic; |
| Chr2 | PQ4 | transcript | 3569664 | 3569816 | ID = TAR55; Note = intergenic; |
| Chr2 | PQ4 | transcript | 4829946 | 4830057 | ID = TAR56; Note = EXON; Parent = AT2G11950.1; |
| Chr2 | PQ4 | transcript | 6088997 | 6089104 | ID = TAR57; Note = EXON; Parent = AT2G14350.1; |
| Chr2 | PQ4 | transcript | 6280599 | 6280961 | ID = TAR58; Note = intergenic; |
| Chr2 | PQ4 | transcript | 6517415 | 6517684 | ID = TAR59; Note = EXON; Parent = AT2G15040.1; |
| Chr2 | PQ4 | transcript | 8779493 | 8779637 | ID = TAR60; Note = intergenic; |
| Chr2 | PQ4 | transcript | 9200271 | 9200454 | ID = TAR61; Note = EXON; Parent = AT2G21460.1; |
| Chr2 | PQ4 | transcript | 9200773 | 9200881 | ID = TAR62; Note = EXON; Parent = AT2G21460.1; |
| Chr2 | PQ4 | transcript | 9875188 | 9875367 | ID = TAR63; Note = intergenic; |
| Chr2 | PQ4 | transcript | 9930150 | 9930427 | ID = TAR64; Note = intergenic; |
| Chr2 | PQ4 | transcript | 12039762 | 12039992 | ID = TAR65; Note = intergenic; |
| Chr2 | PQ4 | transcript | 13057652 | 13057943 | ID = TAR66; Note = EXON; Parent = AT2G30640.1; |
| Chr2 | PQ4 | transcript | 13537101 | 13537248 | ID = TAR67; Note = intergenic; |
| Chr2 | PQ4 | transcript | 13696749 | 13696943 | ID = TAR68; Note = intergenic; |
| Chr2 | PQ4 | transcript | 14190472 | 14190596 | ID = TAR69; Note = intergenic; |
| Chr2 | PQ4 | transcript | 14997744 | 14997896 | ID = TAR70; Note = intergenic; |
| Chr2 | PQ4 | transcript | 15471977 | 15472104 | ID = TAR71; Note = intron; Parent = AT2G36850.1; |
| Chr2 | PQ4 | transcript | 15476590 | 15476699 | ID = TAR72; Note = intergenic; |
| Chr2 | PQ4 | transcript | 15887002 | 15887114 | ID = TAR73; Note = intergenic; |
| Chr2 | PQ4 | transcript | 16026588 | 16026762 | ID = TAR74; Note = intergenic; |
| Chr2 | PQ4 | transcript | 17273007 | 17273262 | ID = TAR75; Note = intergenic; |
| Chr2 | PQ4 | transcript | 17938590 | 17938771 | ID = TAR76; Note = intergenic; |
| Chr2 | PQ4 | transcript | 18173485 | 18173588 | ID = TAR77; Note = intergenic; |
| Chr3 | PQ4 | transcript | 1284836 | 1284978 | ID = TAR78; Note = EXON; Parent = AT3G04717.1; |
| Chr3 | PQ4 | transcript | 1285129 | 1285277 | ID = TAR79; Note = EXON; Parent = AT3G04717.1; |
| Chr3 | PQ4 | transcript | 1745188 | 1745474 | ID = TAR80; Note = EXON; Parent = AT3G05850.1; |
| Chr3 | PQ4 | transcript | 2027119 | 2027229 | ID = TAR81; Note = intron; Parent = AT3G06530.1; |
| Chr3 | PQ4 | transcript | 2031178 | 2031284 | ID = TAR82; Note = intron; Parent = AT3G06530.1; |
| Chr3 | PQ4 | transcript | 2191886 | 2192164 | ID = TAR83; Note = EXON; Parent = AT3G06940.1; |
| Chr3 | PQ4 | transcript | 3963549 | 3963916 | ID = TAR84; Note = EXON; Parent = AT3G12502.1; |
| Chr3 | PQ4 | transcript | 4701930 | 4702221 | ID = TAR85; Note = intergenic; |
| Chr3 | PQ4 | transcript | 5258935 | 5259044 | ID = TAR86; Note = intergenic; |
| Chr3 | PQ4 | transcript | 5838460 | 5838564 | ID = TAR87; Note = EXON; Parent = AT3G17110.1; |
| Chr3 | PQ4 | transcript | 7104312 | 7104478 | ID = TAR88; Note = EXON; Parent = AT3G20365.1; |
| Chr3 | PQ4 | transcript | 7493078 | 7493401 | ID = TAR89; Note = intergenic; |
| Chr3 | PQ4 | transcript | 7864201 | 7864348 | ID = TAR90; Note = intron; Parent = AT3G22240.1; |
| Chr3 | PQ4 | transcript | 8211626 | 8211728 | ID = TAR91; Note = intergenic; |
| Chr3 | PQ4 | transcript | 8937608 | 8937717 | ID = TAR92; Note = intergenic; |
| Chr3 | PQ4 | transcript | 12047115 | 12047292 | ID = TAR93; Note = EXON; Parent = AT3G30416.1; |
| Chr3 | PQ4 | transcript | 12513937 | 12514047 | ID = TAR94; Note = EXON; Parent = AT3G30817.1; |
| Chr3 | PQ4 | transcript | 13373195 | 13373606 | ID = TAR95; Note = EXON; Parent = AT3G32415.1; |
| Chr3 | PQ4 | transcript | 13600838 | 13600960 | ID = TAR96; Note = intergenic; |
| Chr3 | PQ4 | transcript | 13620240 | 13620347 | ID = TAR97; Note = intergenic; |
| Chr3 | PQ4 | transcript | 14810535 | 14810673 | ID = TAR98; Note = intergenic; |
| Chr3 | PQ4 | transcript | 15615992 | 15616134 | ID = TAR99; Note = intergenic; |
| Chr3 | PQ4 | transcript | 15784281 | 15784396 | ID = TAR100; Note = EXON; Parent = AT3G43955.1; |
| Chr3 | PQ4 | transcript | 16223301 | 16223406 | ID = TAR101; Note = intergenic; |
| Chr3 | PQ4 | transcript | 17200993 | 17201244 | ID = TAR102; Note = EXON; Parent = AT3G46660.1; |
| Chr3 | PQ4 | transcript | 18221160 | 18221276 | ID = TAR103; Note = intergenic; |
| Chr3 | PQ4 | transcript | 22060835 | 22061134 | ID = TAR104; Note = EXON; Parent = AT3G59695.1; |
| Chr3 | PQ4 | transcript | 22061356 | 22061467 | ID = TAR105; Note = EXON; Parent = AT3G59695.1; |
| Chr3 | PQ4 | transcript | 22061858 | 22062139 | ID = TAR106; Note = EXON; Parent = AT3G59695.1; |
| Chr3 | PQ4 | transcript | 22561326 | 22561538 | ID = TAR107; Note = EXON; Parent = AT3G60965.1; |
| Chr3 | PQ4 | transcript | 22659806 | 22659978 | ID = TAR108; Note = EXON; Parent = AT3G61185.1; |
| Chr4 | PQ4 | transcript | 1270801 | 1271002 | ID = TAR109; Note = intergenic; |
| Chr4 | PQ4 | transcript | 2191910 | 2192020 | ID = TAR110; Note = EXON; Parent = AT4G04410.1; |
| Chr4 | PQ4 | transcript | 3964456 | 3964678 | ID = TAR111; Note = EXON; Parent = AT4G06708.1; |
| Chr4 | PQ4 | transcript | 6534652 | 6534858 | ID = TAR112; Note = EXON; Parent = AT4G10580.1; |
| Chr4 | PQ4 | transcript | 7512355 | 7512799 | ID = TAR113; Note = intergenic; |
| Chr4 | PQ4 | transcript | 7652127 | 7652246 | ID = TAR114; Note = intron; Parent = AT4G13150.1; |
| Chr4 | PQ4 | transcript | 8036799 | 8037139 | ID = TAR115; Note = EXON; Parent = AT4G13900.1; |
| Chr4 | PQ4 | transcript | 8199493 | 8199599 | ID = TAR116; Note = intergenic; |
| Chr4 | PQ4 | transcript | 8691795 | 8691900 | ID = TAR117; Note = intron; Parent = AT4G15233.1; |
| Chr4 | PQ4 | transcript | 8693266 | 8693442 | ID = TAR118; Note = intron; Parent = AT4G15233.1; |
| Chr4 | PQ4 | transcript | 9694115 | 9694330 | ID = TAR119; Note = intergenic; |
| Chr4 | PQ4 | transcript | 9694696 | 9694808 | ID = TAR120; Note = intergenic; |
| Chr4 | PQ4 | transcript | 9695064 | 9695208 | ID = TAR121; Note = intergenic; |
| Chr4 | PQ4 | transcript | 9696801 | 9696944 | ID = TAR122; Note = intergenic; |
| Chr4 | PQ4 | transcript | 11852077 | 11852219 | ID = TAR123; Note = EXON; Parent = AT4G22505.1; |
| Chr4 | PQ4 | transcript | 11852395 | 11852697 | ID = TAR124; Note = EXON; Parent = AT4G22505.1; |
| Chr4 | PQ4 | transcript | 12284949 | 12285142 | ID = TAR125; Note = intergenic; |
| Chr4 | PQ4 | transcript | 12285391 | 12285816 | ID = TAR126; Note = intergenic; |
| Chr4 | PQ4 | transcript | 12285929 | 12286302 | ID = TAR127; Note = intergenic; |
| Chr4 | PQ4 | transcript | 12473584 | 12473794 | ID = TAR128; Note = intergenic; |
| Chr4 | PQ4 | transcript | 16891478 | 16891793 | ID = TAR129; Note = intergenic; |
| Chr5 | PQ4 | transcript | 84435 | 84592 | ID = TAR130; Note = EXON; Parent = AT5G01215.1; |
| Chr5 | PQ4 | transcript | 1164586 | 1164766 | ID = TAR131; Note = EXON; Parent = AT5G04235.1; |
| Chr5 | PQ4 | transcript | 1166221 | 1166360 | ID = TAR132; Note = EXON; Parent = AT5G04235.1; |
| Chr5 | PQ4 | transcript | 4150542 | 4150679 | ID = TAR133; Note = intron; Parent = AT5G13080.1; |
| Chr5 | PQ4 | transcript | 4709525 | 4709773 | ID = TAR134; Note = intergenic; |
| Chr5 | PQ4 | transcript | 5304974 | 5305082 | ID = TAR135; Note = EXON; Parent = AT5G16235.1; |
| Chr5 | PQ4 | transcript | 5305601 | 5305740 | ID = TAR136; Note = EXON; Parent = AT5G16235.1; |
| Chr5 | PQ4 | transcript | 5771411 | 5771522 | ID = TAR137; Note = intron; Parent = AT5G17510.1; |
| Chr5 | PQ4 | transcript | 5893629 | 5893746 | ID = TAR138; Note = intron; Parent = AT5G17830.1; |
| Chr5 | PQ4 | transcript | 6937393 | 6937568 | ID = TAR139; Note = intergenic; |
| Chr5 | PQ4 | transcript | 7828239 | 7828571 | ID = TAR140; Note = EXON; Parent = AT5G23240.1; |
| Chr5 | PQ4 | transcript | 8213452 | 8213665 | ID = TAR141; Note = EXON; Parent = AT5G24206.1; |
| Chr5 | PQ4 | transcript | 8218090 | 8218204 | ID = TAR142; Note = intron; Parent = AT5G24210.1; |
| Chr5 | PQ4 | transcript | 8958642 | 8958862 | ID = TAR143; Note = intergenic; |
| Chr5 | PQ4 | transcript | 10019022 | 10019176 | ID = TAR144; Note = intergenic; |
| Chr5 | PQ4 | transcript | 10019359 | 10019652 | ID = TAR145; Note = intergenic; |
| Chr5 | PQ4 | transcript | 13105430 | 13105592 | ID = TAR146; Note = EXON; Parent = AT5G34851.1; |
| Chr5 | PQ4 | transcript | 14567664 | 14567777 | ID = TAR147; Note = intergenic; |
| Chr5 | PQ4 | transcript | 14906568 | 14906819 | ID = TAR148; Note = intergenic; |
| Chr5 | PQ4 | transcript | 15649299 | 15649545 | ID = TAR149; Note = intergenic; |
| Chr5 | PQ4 | transcript | 16054202 | 16054301 | ID = TAR150; Note = EXON; Parent = AT5G40060.1; |
| Chr5 | PQ4 | transcript | 16745478 | 16745587 | ID = TAR151; Note = intergenic; |
| Chr5 | PQ4 | transcript | 17152037 | 17152359 | ID = TAR152; Note = intergenic; |
| Chr5 | PQ4 | transcript | 17971508 | 17971660 | ID = TAR153; Note = intergenic; |
| Chr5 | PQ4 | transcript | 18002454 | 18002633 | ID = TAR154; Note = intergenic; |
| Chr5 | PQ4 | transcript | 19159268 | 19159412 | ID = TAR155; Note = intergenic; |
| Chr5 | PQ4 | transcript | 19873174 | 19873392 | ID = TAR156; Note = EXON; Parent = AT5G48965.1; |
| Chr5 | PQ4 | transcript | 19873538 | 19873653 | ID = TAR157; Note = EXON; Parent = AT5G48965.1; |
| Chr5 | PQ4 | transcript | 20921928 | 20922221 | ID = TAR158; Note = intergenic; |
| Chr5 | PQ4 | transcript | 21549835 | 21549947 | ID = TAR159; Note = intergenic; |
| Chr5 | PQ4 | transcript | 21717504 | 21717608 | ID = TAR160; Note = intergenic; |
| Chr5 | PQ4 | transcript | 22355958 | 22356226 | ID = TAR161; Note = EXON; Parent = AT5G55050.1; |
| Chr5 | PQ4 | transcript | 22479122 | 22479371 | ID = TAR162; Note = EXON; Parent = AT5G55420.1; |
| Chr5 | PQ4 | transcript | 22479505 | 22479770 | ID = TAR163; Note = intergenic; |
| Chr5 | PQ4 | transcript | 23766482 | 23766590 | ID = TAR164; Note = EXON; Parent = AT5G58810.1; |
| Chr5 | PQ4 | transcript | 24186552 | 24186699 | ID = TAR165; Note = EXON; Parent = AT5G60022.1; |
| Chr5 | PQ4 | transcript | 24186836 | 24187127 | ID = TAR166; Note = EXON; Parent = AT5G60022.1; |
| Chr5 | PQ4 | transcript | 24279446 | 24279822 | ID = TAR167; Note = EXON; Parent = AT5G60285.1; |
| Chr5 | PQ4 | transcript | 24422361 | 24422543 | ID = TAR168; Note = intergenic; |
| Chr5 | PQ4 | transcript | 24788305 | 24788415 | ID = TAR169; Note = intergenic; |
| ChrM | PQ4 | transcript | 40160 | 40440 | ID = TAR170; Note = intergenic; |
| ChrM | PQ4 | transcript | 245983 | 246093 | ID = TAR171; Note = intergenic; |
| ChrM | PQ4 | transcript | 246645 | 246745 | ID = TAR172; Note = intergenic; |
| ChrM | PQ4 | transcript | 286404 | 286544 | ID = TAR173; Note = intergenic; |
| ChrM | PQ4 | transcript | 288554 | 288724 | ID = TAR174; Note = intron; Parent = ATMG01120.1; |
| ChrM | PQ4 | transcript | 316831 | 316937 | ID = TAR175; Note = intergenic; |

### EXAMPLES

The following examples are provided for the purpose of illustrating the present invention and should in no way be interpreted as limiting the scope thereof.

### Example 1

### Identification of OSIP Genes in A. thaliana Using Tiling Arrays

### Plant Material, Growth Conditions and Treatments

Seeds of Arabidopsis thaliana wild-type ecotype Columbia-0 (Col-0) were obtained from the European A. thaliana stock Centre (NASC; http://nasc.nott.ac.uk/home.html). After sowing, they were grown in a growth chamber with a 21° C. daytime temperature, 18° C. night-time temperature, 75% humidity and a 12 h day-light cycle with a light intensity of approximately 120 μmol/m² s.

Rosette leaves from soil-grown A. thaliana plants were sprayed with paraquat (25 μM methylviologendichlorodehydrate 98%) to obtain an equal and proportional distribution. Control plants were mock treated by spraying with H₂O. The treated plants were then kept at 100% relative humidity for 48 hours. Then, all plant material located above ground surface was harvested in liquid nitrogen and stored at −80° C. prior to further analysis.

### Isolation of Total RNA

RNA was isolated from approximately 100 mg plant material per sample by the guanidinium isothiocyanate method (TRIzol reagent; Invitrogen, Carlsbad, USA) followed by purification using the RNeasy Mini Kit (Qiagen, Valencia, USA) according to the manufacturers' instructions.

### Target Preparation and Tiling Array Hybridization

The mRNA samples were labeled and hybridized onto GeneChip® Arabidopsis Tiling 1.0R Arrays (Affymetrix, Santa Clara, USA). Two independent biological samples were used for each condition to create biological repeats. Scanning and image processing were performed using Affymetrix equipment and software.

### Identification of Short Unannotated and Paraquat-Induced Genomic Regions

Background correction, log₂ transformation and interslide normalization were performed with the Affymetrix Tiling Analysis Software—Version 1.1, Build 2. The resulting tiling array data (consisting of chromosome positions of probes linked to normalized log₂ transformed intensities for both paraquat and control samples), the analysis results and all required information (e.g. A. thaliana genome annotation) were stored in a MySQL database. Since the tiling arrays were based on the A. thaliana genome sequence of the 2007's version of the TAIR database (20), the corresponding TAIR7 genome annotation was used for the analysis of the tiling array data.

Selection and identification of transcriptionally active regions included the following steps:

(i) intensities of biological replicates were combined and a threshold value of 8.6 was chosen to identify transcriptionally active probes based on both the tiling array intensities of genomic regions with known expression status during the paraquat and control treatment and the results of control qRT-PCR experiments. Individual probes with intensities below the threshold that were surrounded by transcriptionally active probes were considered active and vice versa to remove the effect of probes with poor hybridization properties.
(ii) groups of 4-13 successive transcriptionally active probes were combined into short transcriptionally active regions. Selection of even smaller regions has several disadvantages, including a high number of false positives and the identification of very small genes that are difficult to amplify in later stages. All active regions of 14-15 successive probes were already annotated.
(iii) transcriptionally active regions were withheld if the number of transcriptionally active probes was smaller in the control than in the paraquat treatment and if the average intensity of all probes in the smallest of the two regions was at least 1.4 higher in the paraquat treatment than in the control. Both rules were based on the results for genomic regions that were known to be induced upon paraquat treatment. The former rule was added because of the frequent occurrence of probes having zero intensity in one of the four hybridizations. In small regions these zero values can have substantial influence on the average intensity leading to false positive calls.
(iv) each selected region was compared to the A. thaliana TAIR7 genome annotation and only regions that were located in intergenic regions, introns or pseudogenes were withheld.

### Selection of Open Reading Frames

Using the TAIR7 genome sequence, a fasta file containing the corresponding DNA sequences of all selected short unannotated and paraquat-induced regions were retrieved. On each side of the active region an extra sequence of 50 bp was selected to minimize the chance of missing the start or stop codon.

### Results

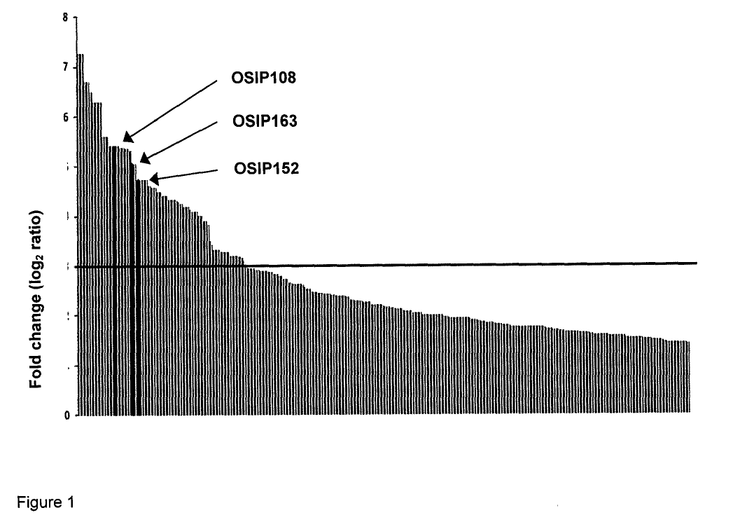

Leaf samples were collected at 48 h after paraquat treatment from both 4-weeks old treated A. thaliana Col0 plants and their corresponding control plants upon mock-treatment with water. Two biological replicates were performed. From all plant samples high quality mRNA was isolated, labeled and used for hybridization on GeneChip® Arabidopsis tiling 1.0R arrays (Affymetrix). Log₂ transformed normalized microarray data of the two biological replicates were combined and used to identify transcriptionally active regions (TARs) in the Arabidopsis genome based on a threshold that was defined using control genomic regions with known expression status in the treatments under study. This way, a total of 92,844 and 86,272 TARs were identified in the paraquat and the control data set respectively. Short unannotated TARs that were induced by the paraquat treatment as compared to the control treatment were selected, again using a threshold that was based on the transcriptional behaviour of a set of appropriate controls, resulting in the identification of 176 Oxidative Stress Induced Peptides (OSIP)-encoding regions. The bulk of these TARs were intergenic, although 70 and 18 OSIP-encoding regions were located in pseudogenes and introns of annotated genes respectively. The corresponding DNA sequences of the selected TARs were translated in the six possible reading frames with the ExPaSy translate tool of the Swiss Institute of Bioinformatics (http://www.expasy.ch/tools/dna.html). Open reading frames (ORFs) of at least 30 bp (10 amino acids) were retained, resulting in a list of 575 potential OSIP-encoding genes, ordered based on their induction level (FIG. 1, Table 1). Peptide sequences shorter than 10 amino acids or located entirely in the outer 50 bp were discarded.

### Example 2

### Overexpression of OSIPs Results in Increased Peroxide MIC Values of Yeast

### Primer Design

The DNA sequences of the open reading frames were extended with 50 extra nucleotides after the stop codon to increase the choice possibilities for the reverse primer. The design of specific primers for each selected ORF was done with Primer3 software (21). To allow for transformation using Gateway® technology the sequence CACCAAAA was added to each forward primer.

### Materials and Yeast Strains

Yeast strain used was Saccharomyces cerevisiae strain BY4741 (wild-type, WT) (Euroscarf, Germany), which was cultivated in Yeast Peptone Dextrose (YPD) (1% yeast extract, 2% peptone, 2% glucose). Yeast strains transformed with the pYES-DEST52 Gateway® vector (Invitrogen, Carlsbad, USA) were cultured in SC-ura (0.8 g/l CSM-ura, complete amino acid supplement mixture minus uracil, Bio 101 Systems; 6.5 g/l YNB, yeast nitrogen base; 20 g/l glucose). Oxidative stress induced peptides (OSIPs) were synthesized by the addition of galactose to the growth medium.

### Construction of a Mini Library in Yeast

The coding sequences of the potential OSIP genes were cloned into the pENTR™ plasmid (Invitrogen, Carlsbad, USA) using the pENTR™/D-TOPO® cloning system according to the manufacturer's instructions (Invitrogen) and the presence of each ORF in the resulting pool of E. coli transformants was investigated by PCR with the individual primers. The inserts were then transferred to the pYES-DEST52 Gateway® vector and subsequently used for transformation to Sacharomyces cerevisiae BY4741 using the Gietz protocol (22).

Oxidative Stress Assays in Yeast

Oxidative stress tolerance of individual yeast transformants was examined by determination of de minimal inhibitory concentration (MIC) for H₂O₂, using a twofold dilution serie (0-20 mM) in SC-ura (Gal) and 1/200 yeast inoculum of an overnight culture in SC-ura (Glu).

To investigate an active role in oxidative stress resistance, the full coding sequences of the potential OSIP genes were individually amplified by PCR, pooled, and used for transformation of yeast using the pENTR™/D-TOPO® cloning system (Invitrogen, Carlsbad, USA). Overexpression of the OSIP gene was activated by the addition of galactose to the growth medium. As paraquat induces oxidative stress in yeast during respiration, thus upon cultivation in glycerol, it was not possible to screen for yeast transformants with increased tolerance against paraquat. Therefore, we screened the corresponding yeast transformants for improved tolerance against oxidative stress induced by peroxide in galactose-containing medium by determining the minimal inhibitory concentration (MIC) of peroxide. As positive controls, we cloned two known A. thaliana peptides that govern oxidative stress tolerance in gist, namely LEA5 and AtMtATP6 (8, 9). To this end, for at least 500 individual yeast transformants. In this way, we selected 9 yeast transformants that were characterized by a consistent 2-fold increased peroxide MIC value in galactose-containing medium (MIC=5 mM peroxide), compared to the other yeast transformants (MIC=2.5 mM peroxide). A similar increase in MIC value was observed for yeast transformants overexpressing LEA5 or AtMtATP6.

Sequence analysis revealed that these nine transformants contained seven different constructs, with two constructs occurring twice in our selection. The sequences of these seven constructs, SIP108_{—}4 (further SIP108 or OSIP108), SIP152_{—}2 (further SIP152 or OSIP152), SIP163_{—}3 (further SIP163 or OSIP163), SIP14_{—}2, SIP14_{—}1, SIP11_{—}0 (further SIP11 or OSIP11) and SIP37_{—}1 (further SIP37 or OSIP37) are shown in Table 3 and 4.

The log₂ ratios of OSIP108, OSIP163 and OSIP152 upon paraquat treatment in the tiling array were 5.42; 5.07 and 4.74, respectively. To validate these expression data, we next assessed expression levels of the selected 3 OSIPs using qRT-PCR analysis under the same conditions and at the same time point as used in the tiling array analysis. The log₂ ratios of OSIP108, OSIP163 and OSIP152 assessed were 5.32, 5.68 and 5.00, respectively. Hence, the resulting qRT-PCR data are in line with the normalized log ratios from the tiling arrays, thereby confirming the observed gene expression patterns.

### Example 3

### Overexpression of OSIP108 Governs Oxidative Stress Tolerance in Yeast

Resistance of the selected yeast transformants and the two positive control transformants against oxidative stress induced by H₂O₂ was additionally assayed in a halo test, by spotting 15 μL of 240 mM H₂O₂ on SC-ura agar, containing either glucose or galactose, and inoculated with 1/200 of overnight yeast culture as described previously (23). After 48 h of incubation at 30° C., diameters of the halos were measured.

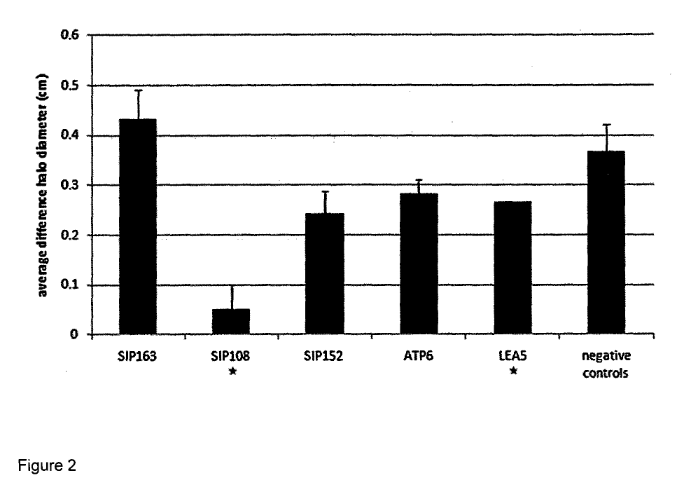

The halo-test comprises a predefined but continuous concentration gradient of H₂O₂, applied to inoculated agar plates. Upon incubation, circles (halos) of growth inhibition are visible. By comparing the diameter of the halos of plates with yeast grown on glucose (not allowing for expression of the OSIP) and galactose (inducing overexpression of the OSIP) the effect of the OSIP on oxidative stress tolerance was quantified. The average difference in halo diameter obtained on galactose-containing agar inoculated with OSIP108-overexpressing yeast was significantly lower as compared to yeast transformants overexpressing negative controls or SIP152 or SIP163 (FIG. 2). SIP14_{—}2, SIP14_{—}1 and SIP11 also showed significant enhanced tolerance in this assay.

### Example 4

### Exogenous Application of OSIP108 to Yeast Cultures Reduces the ROS Accumulation and Increases Survival Upon Peroxide Treatment

### ROS Accumulation Assay and Survival Assay

ROS, after H₂O₂ treatment in the absence or presence of 500 μM OSIP, were determined using 2,7-dichlorodihydrofluorescein diacetate (H₂DCFDA) staining as described previously (24). Survival of WT yeast in PBS following H₂O₂ treatment in the absence or presence of 500 μM OSIP was assessed as described (25).

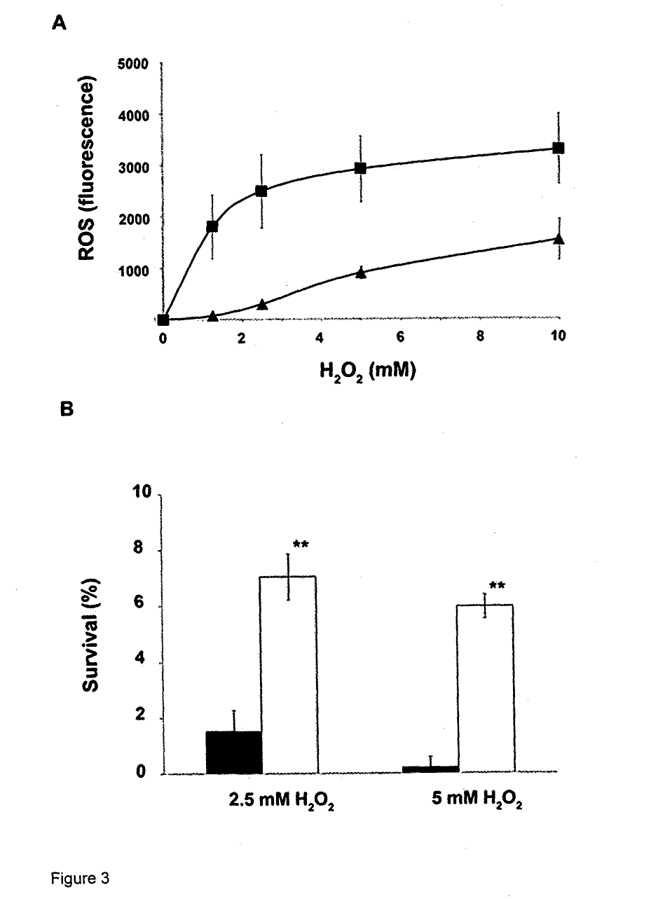

The effect of OSIP108 on endogenous ROS levels induced by peroxide in yeast were evaluated. Peroxide-induced ROS in yeast in the absence and presence of OSIP108 were measured using 2,7-dichlorodihydrofluorescein diacetate (H₂DCFDA) (FIG. 3A). The fluorescence intensity of 2′,7′-dichlorofluorescein (DCF) yeast cultures treated with different concentrations of peroxide was significantly lower in the presence of 500 μM OSIP108 compared to the mock treatment. Moreover, we found the survival of peroxide-treated yeast cells (5 mM) to increase 27-fold upon incubation with 500 μM OSIP108 (FIG. 3B).

SIP14_{—}2, SIP14_{—}1 and SIP11 also resulted in a decrease in the amount of endogenous ROS in the yeast cells. However, they showed less effect than SIP108 on the survival of the yeast cells upon addition of the peptides and after treatment with peroxide.

### Example 5

### Effect of OSIP108 on Yeast Growth Inhibition Induced by Apoptosis-Inducing Compounds

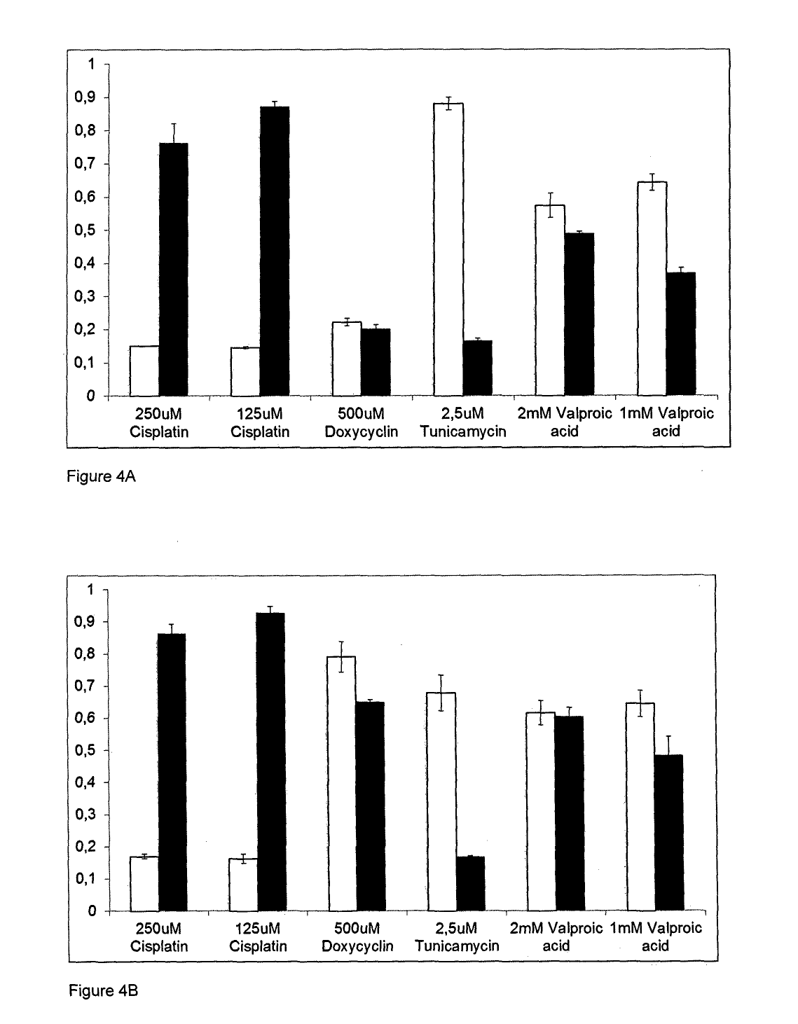

As induction of oxidative stress and apoptosis are closely linked, we investigated the effect of OSIP108 and its inactive variant OSIP(C3A) on Saccharomyces cerevisiae growth in the presence of various apoptosis-inducers and mitochondrial toxins like copper, valproic acid, tunicamycin, doxycyclin and cisplatin. In this respect, in parallel with mitochondrial copper toxicity, the use of the other mitochondrial toxins, in casu valproic acid, tunicamycin, doxycyclin and cisplatin, expand the range of causes of mitochondrial dysfunction and help find a broader basis for compounds rescuing mitochondrial dysfunction. Optical density (OD600) of yeast BY4741 was monitored upon treatment with different apoptosis-inducing compounds in the presence or absence of 200 μM OSIP108. OSIP108 and apoptosis-inducers were added simultaneously to the yeast cells. Percentage growth was calculated as the ratio of the OD600 of the treated culture over an unstressed yeast culture after 10 h or 48 h of incubation. Treatment of yeast cells with 200 uM OSIP108 results in significant increase in growth, compared to control treatment, in presence of 250 and 125 μM cisplatin whereas OSIP108 fails to rescue yeast growth inhibition upon treatment with other apoptosis-inducers (FIG. 4). Also, OSIP108 but not OSIP(C3A) can rescue copper- and cisplatin-induced growth inhibition of yeast

### Example 6

### Exogenous Application of OSIP108 to A. thaliana Leaves Increases Paraquat Tolerance

At least twelve soil grown, 4 week old Col-0 A. thaliana plants, were used in which one leaf of each plant was syringe-infiltrated with 100 μM OSIP108 dissolved in 1% dimethyl sulphoxide (DMSO). At least 12 control plants were mock treated with 1% DMSO only. After 24 h incubation in the growth chamber, the plants were inoculated with a drop of 5 μl paraquat (100 μM). The lesion diameters caused by the paraquat treatment were measured and compared 2 and 3 days after the treatment.

Three days after paraquat application, the average lesion diameter of plants pretreated with OSIP108 (2.18±1.60 mm, p=0.002) was significantly lower than the average lesion diameter of the control plants (4.45±1.37 mm). These data confirm the ability of OSIP108 to increase oxidative stress tolerance not only in yeast, but also in plants.

### Example 7

### Effect of OSIP108 on Cell Survival of HepG2 Cells in the Presence of Copper or Cisplatin

The human hepatoma cell line HepG2 which naturally expresses hATP7B was incubated with 100 μM of peptide OSIP108. The viability of cells was determined after 48 h exposure to toxic copper concentrations of 0.75 mM by MTT assay. The viability was calculated relative to cells that receive no copper (100%).

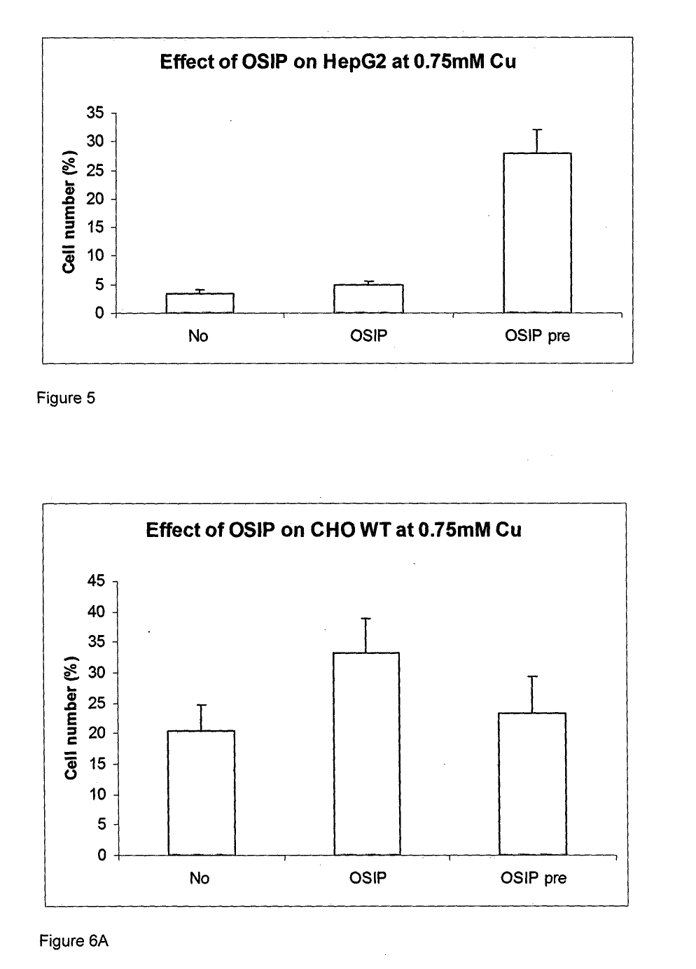

Pre-incubation of the hepatoma cell line HepG2 with OSIP108 resulted in increased survival of the cells in the presence of copper or cisplatin (FIG. 5). The inactive OSIP108(C3A) was used as a negative control.

### Example 8

### Effect of Endogenous or Exogenous Application of OSIP108 on Copper-Induced Growth Inhibition of Δccc2

Copper ions are known to induce apoptosis of hepatocytes, one of the underlying mechanisms of copper-induced toxicity in Wilson's Disease (WD). WD is caused by mutations in the ATPase ATP7b. We found that overexpression of the gene encoding OSIP108 in a yeast Δccc2 mutant, which is devoid of the ortholog of ATP7b, could alleviate copper-induced growth inhibition of this mutant.

Exogenous addition of OSIP108 to Δccc2 yeast mutant cultures in the presence of a lethal copper dose also induces survival of the yeast mutant. OSIP108 reduced the copper-induced accumulation of reactive oxygen species (ROS) and increased viability of the yeast mutant. This was tested by spotting 5 μL of 10 mM OSIP108 onto agar plates containing a yeast Δccc2 mutant, 500 μM CuSO₄ and a viability dye based on MTT. Plates were incubated for 24 h and placed at 4° C. for 2 days. Blue halos were indicative of surviving yeast cells.

### Example 9

### Effect of OSIP108 on Wilson's Disease Model Cell Lines

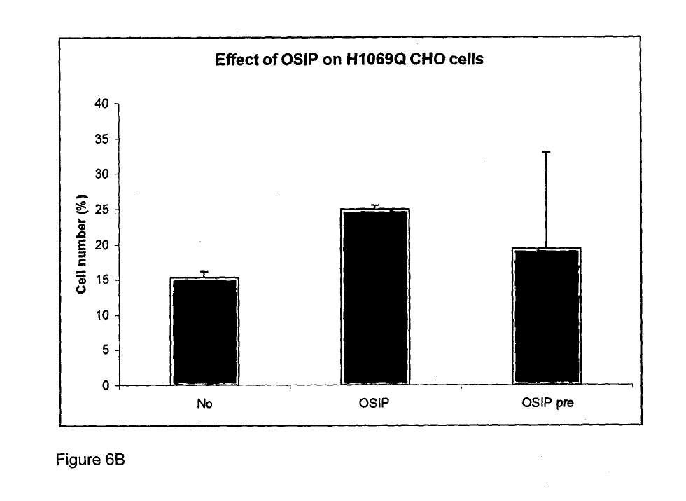

CHO cell lines expressing either ATP7b or ATP7b mutant H1069Q (a mutation frequently found in patients having Wilson's Disease) were incubated with OSIP108 (100 μM). The viability of cells was determined after 48 h exposure to toxic copper concentrations of 0.75 mM by MTT assay and calculated relative to cells that receive no copper (100%). Incubation of CHO cell lines expressing either ATP7b or ATP7b mutant H1069Q with OSIP108 enhanced the copper resistance of the cell lines and increased their viability in the presence of copper (FIG. 6)

### REFERENCES

1. Berliner, J. A., Navab, M., Fogelman, A. M., Frank, J. S., Demer, L. L., Edwards, P. A., Watson, A. D., and Lusis, A. J. (1995) Circulation 91, 2488-2496
2. Dexter, D. T., Carter, C. J., Wells, F. R., Javoy-Agid, F., Agid, Y., Lees, A., Jenner, P., and Marsden, C. D. (1989) J Neurochem 52, 381-389
3. Low, P. S., and Merida, J. R. (1996) Physiologia Plantarum 96, 533-542
4. Smith, M. A., Perry, G., Richey, P. L., Sayre, L. M., Anderson, V. E., Beal, M. F., and Kowall, N. (1996) Nature 382, 120-121
5. Lee, C. Y., Lee, C. H., Shih, C. C., and Liou, H. H. (2008) Biochem Pharmacol 76, 1155-1164
6. Yang, W., and Tiffany-Castiglioni, E. (2005) J Toxicol Environ Health A 68, 1939-1961
7. Cunningham, T. J., Jing, H., Akerblom, I., Morgan, R., Fisher, T. S., and Neveu, M. (2002) Exp Neurol 177, 32-39
8. Zhang, X., Liu, S., and Takano, T. (2008) Biotechnol Lett 30, 1289-1294
9. Mowla, S. B., Cuypers, A., Driscoll, S. P., Kiddie, G., Thomson, J., Foyer, C. H., and Theodoulou, F. L. (2006) Plant J 48, 743-756
10. Fromenty, B., Robin M. A., Igoudjil, A., Mansouri, A., Pessayre, D. (2004), Diabetes Metab 30: 121-138.
11. Krens et al. (1982) Nature 296, 72-74
12. Shillito et al. (1985) Bio/Technol. 3, 1099-1102
13. Crossway et al. (1986) Mol. Gen. Genet. 202, 179-185
14. Klein et al. (1978) Nature 327, 70
15. Caruthers et al. (1982) Cold Spring Harbor Symp. Quant. Biol. 47, 411-418
16. Adams et al. (1983) J. Am. Chem. Soc. 105, 661.
17. Buchman and Berg (1988) Mol. Cell. biol. 8: 4395-4405
18. Callis et al. (1987) Genes Dev 1:1 183-1200)
20. Swarbreck, D., Wilks, C., Lamesch, P., Berardini, T. Z., Garcia-Hernandez, M., Foerster, H., Li, D., Meyer, T., Muller, R., Ploetz, L., Radenbaugh, A., Singh, S., Swing, V., Tissier, C., Zhang, P., and Huala, E. (2008) Nucleic Acids Res 36, D1009-1014
21. Rozen, S., and Skaletsky, H. (2000) Methods Mol Biol 132, 365-386
22. Gietz, R. D., Schiestl, R. H., Willems, A. R., and Woods, R. A. (1995) Yeast 11, 355-360
23. Aerts, A. M., Francois, I. E., Bammens, L., Cammue, B. P., Smets, B., Winderickx, J., Accardo, S., De Vos, D. E., and Thevissen, K. (2006) FEBS Lett 580, 1903-1907
24. Francois, I. E., Bink, A., Vandercappellen, J., Ayscough, K. R., Toulmay, A., Schneiter, R., van Gyseghem, E., Van den Mooter, G., Borgers, M., Vandenbosch, D., Coenye, T., Cammue, B. P., and Thevissen, K. (2009) J Biol Chem 284, 32680-32685
25. Bink, A., Govaert, G., Francois, I. E., Pellens, K., Meerpoel, L., Borgers, M., Van Minnebruggen, G., Vroome, V., Cammue, B. P., and Thevissen, K. (2010) FEMS Yeast Res 10, 812-818

1. An isolated peptide comprising an amino acid sequence with at least 70% sequence identity to amino acid sequence SEQ ID No. 1; wherein the isolated peptide increases the oxidative stress tolerance of a cell or organism under oxidative stress conditions.

2. The isolated peptide according to claim 1, wherein the isolated peptide increases the viability of cell under oxidative stress conditions or in the presence of an agent inducing mitochondrial dysfunction.

3. An isolated nucleic acid encoding for the peptide according to claim 1.

4. The isolated nucleic acid according to claim 3 comprising a nucleotide sequence with at least 70% sequence identity to nucleotide sequence SEQ ID NO:576.

5. A genetic construct comprising the following operably linked DNA elements: (a) a nucleic acid according to claim 3; (b) one or more control sequences capable of driving expression of said nucleic acid; and (c) a 3′ end region comprising a transcription termination sequence.

6. A method for increasing the oxidative stress tolerance of a cell or organism comprising contacting or transfecting said cell or organism with a peptide according to claim 1, a peptidomimetic thereof or a nucleic acid encoding therefor.

7. The method according to claim 6, comprising introducing and expressing into a cell or non-human organism a genetic construct according to claim 5.

8. The method according to claim 6 wherein said cell or organism is an eukaryotic cell or organism.

9. The method according to claim 8 wherein said cell or organism is a plant cell or plant, or an eukaryotic microbial cell or organism.

10. A transgenic cell or non-human organism having increased tolerance to oxidative stress or having increased viability under oxidative stress conditions or in the presence of an agent inducing mitochondrial dysfunction relative to the corresponding wild-type cell or non-human organism; wherein said transgenic cell or non-human organism is transfected with and expresses the genetic construct according to claim 5.

11. The transgenic cell or non-human organism according to claim 10 wherein said cell or organism is a plant cell or plant or part thereof, or an eukaryotic microbial cell or organism.

12. Harvestable parts of the transgenic plant according to claim 11.

13. A method for the treatment and/or prevention of an oxidative stress related disorder comprising administering to a mammal peptide according to claim 1 or peptidemimetic thereof.

14. The method according to claim 13 wherein said oxidative stress related disorder is a mitochondrial dysfunction related disorder.

15. A pharmaceutical composition comprising (i) a peptide according to claim 1, or peptidomimetic thereof and (ii) one or more pharmaceutically acceptable compounds, carriers and/or adjuvants.
